(19) United States
(12) Patent Application Publication
(10) Pub. No.: US 2010/0016340 A1
(43) Pub. Date: Jan. 21, 2010

(54) PHARMACEUTICAL COMPOUNDS

(75) Inventors: Steven John Woodhead, Cambridge (GB); Christopher Hamlett, Cambridge (GB); Hannah Fiona Sore, Cambridge (GB); David Winter Walker, Linton (GB); Marinus Leendert Verdonk, Burwell (GB); Ian Collins, Redhill (GB); John Caldwell, Sutton (GB); Kwai-Ming Cheung, Staines (GB); Tatiana Faria Da Fonseca McHardy, Sunbury-on-Thames (GB)

Correspondence Address: HESLIN ROTHENBERG FARLEY & MESITI PC, 5 COLUMBIA CIRCLE, ALBANY, NY, 12203, US

(73) Assignees: Astex Therapeutics Limited, Cambridge (GB); The Institute of Cancer Research: Royal Cancer Hospital, London (GB); Cancer Research Technology Limited, London (GB); Astrazeneca AB, Sodertalje (SE)

(21) Appl. No.: 12/298,338

(22) PCT Filed: Apr. 25, 2007
(86) PCT No.: PCT/GB07/01522

§ 371 Date: Oct. 24, 2008

Related U.S. Application Data
(60) Provisional application No. 60/745,558, filed on Apr. 25, 2006.
(60) Provisional application No. 60/871,382, filed on Dec. 21, 2006.

(30) Foreign Application Priority Data
Apr. 25, 2006 (GB) 0608184.8

(51) Int. Cl. A61K 31/522; C07D 401/14; A61K 31/437; A61K 31/519; A61P 35/00

(52) U.S. Cl. 514/263.22; 546/113; 514/300; 544/280; 514/265.1; 544/276

(57) ABSTRACT

The invention provides compounds of the formula (I):

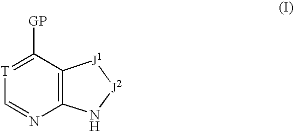

or salts, solvates, tautomers or N-oxides thereof, wherein J¹-J² is CH═CH, N═CH, CH═N, HN—C(O) or CH₂CO; T is N or CH and GP is as defined in the claims. The compounds have activity as inhibitors of PKA and PKB kinases and are useful in the treatment of cancers.

This invention relates to purine, purinone and deazapurine and deazapurinone compounds or structural isomers thereof that inhibit or modulate the activity of protein kinase B (PKB) and/or protein kinase A (PKA), to the use of the compounds in the treatment or prophylaxis of disease states or conditions mediated by PKB and/or PKA, and to novel compounds having PKB and/or PKA inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

Apoptosis or programmed cell death is an important physiological process which removes cells no longer required by an organism. The process is important in early embryonic growth and development allowing the non-necrotic controlled breakdown, removal and recovery of cellular components. The removal of cells by apoptosis is also important in the maintenance of chromosomal and genomic integrity of growing cell populations. There are several known checkpoints in the cell growth cycle at which DNA damage and genomic integrity are carefully monitored. The response to the detection of anomalies at such checkpoints is to arrest the growth of such cells and initiate repair processes. If the damage or anomalies cannot be repaired then apoptosis is initiated by the damaged cell in order to prevent the propagation of faults and errors. Cancerous cells consistently contain numerous mutations, errors or rearrangements in their chromosomal DNA. It is widely believed that this occurs in part because the majority of tumours have a defect in one or more of the processes responsible for initiation of the apoptotic process. Normal control mechanisms cannot kill the cancerous cells and the chromosomal or DNA coding errors continue to be propagated. As a consequence restoring these pro-apoptotic signals or suppressing unregulated survival signals is an attractive means of treating cancer.

The signal transduction pathway containing the enzymes phosphatidylinositol 3-kinase (PI3K), PDK1 and PKB amongst others, has long been known to mediate increased resistance to apoptosis or survival responses in many cells. There is a substantial amount of data to indicate that this pathway is an important survival pathway used by many growth factors to suppress apoptosis. The enzymes of the PI3K family are activated by a range of growth and survival factors e.g. EGF, PDGF and through the generation of polyphosphatidylinositols, initiates the activation of the downstream signalling events including the activity of the kinases PDK1 and protein kinase B (PKB) also known as akt. This is also true in host tissues, e.g. vascular endothelial cells as well as neoplasias. PKB is a protein ser/thr kinase consisting of a kinase domain together with an N-terminal PH domain and C-terminal regulatory domain. The enzyme PKBₐₗₚₕₐ (akt1) itself is phosphorylated on Thr 308 by PDK1 and on Ser 473 by ‘PDK2’ now believed to be constituted from the target of rapamycin (TOR) kinase and its associated protein rictor. Full activation requires phosphorylation at both sites whilst association between PIP3 and the PH domain is required for anchoring of the enzyme to the cytoplasmic face of the lipid membrane providing optimal access to substrates.

At least 10 kinases have been suggested to function as a Ser 473 kinase including mitogen-activated protein (MAP) kinase-activated protein kinase-2 (MK2), integrin-linked kinase (ILK), p38 MAP kinase, protein kinase Calpha (PKCalpha), PKCbeta, the NIMA-related kinase-6 (NEK6), the mammalian target of rapamycin (mTOR), the double-stranded DNA-dependent protein kinase (DNK-PK), and the ataxia telangiectasia mutated (ATM) gene product. Available data suggest that multiple systems may be used in cells to regulate the activation of PKB. Full activation of PKB requires phosphorylation at both sites whilst association between PIP3 and the PH domain is required for anchoring of the enzyme to the cytoplasmic face of the lipid membrane providing optimal access to substrates.

Recently, it has been reported that somatic mutations within the PI3K catalytic subunit, PIK3CA, are common (25-40%) among colorectal, gastric, breast, ovarian cancers, and high-grade brain tumors. PIK3 CA mutations are a common event that can occur early in bladder carcinogenesis. In invasive breast carcinomas, PIK3CA alterations are mainly present in lobular and ductal tumours. The PI3K pathway is extensively activated in endometrial carcinomas, and that combination of PIK3CA/PTEN alterations might play an important role in development of these tumors. Tumours activated by mutations of PI3 kinase and loss of PTEN will have sustained activation of PKB and will be as a result disproportionately sensitive to inhibition by PKA/PKB inhibitors.

Activated PKB in turns phosphorylates a range of substrates contributing to the overall survival response. Whilst we cannot be certain that we understand all of the factors responsible for mediating the PKB dependent survival response, some important actions are believed to be phosphorylation and inactivation of the pro-apoptotic factor BAD and caspase 9, phosphorylation of Forkhead transcription factors e.g. FKHR leading to their exclusion from the nucleus, and activation of the NfkappaB pathway by phosphorylation of upstream kinases in the cascade.

In addition to the anti-apoptotic and pro-survival actions of the PKB pathway, the enzyme also plays an important role in promoting cell proliferation. This action is again likely to be mediated via several actions, some of which are thought to be phosphorylation and inactivation of the cyclin dependent kinase inhibitor of p21^{CiP1/WAF1}, and phosphorylation and activation of mTOR, a kinase controlling several aspects of cell size, growth and protein translation.

The phosphatase PTEN which dephosphorylates and inactivates polyphosphatidyl-inositols is a key tumour suppressor protein which normally acts to regulate the PI3K/PKB survival pathway. The significance of the PI3K/PKB pathway in tumourigenesis can be judged from the observation that PTEN is one of the most common targets of mutation in human tumours, with mutations in this phosphatase having been found in ˜50% or more of melanomas (Guldberg et al 1997, Cancer Research 57, 3660-3663) and advanced prostate cancers (Cairns et al 1997 Cancer Research 57, 4997). These observations and others suggest that a wide range of tumour types are dependent on the enhanced PKB activity for growth and survival and would respond therapeutically to appropriate inhibitors of PKB.

There are 3 closely related isoforms of PKB called alpha, beta and gamma (AKT1, 2 and 3), which genetic studies suggest have distinct but overlapping functions. Evidence suggests that they can all independently play a role in cancer. For example PKB beta has been found to be over-expressed or activated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330), PKB alpha is amplified in human gastric, prostate and breast cancer (Staal 1987, PNAS 84, 5034-5037; Sun et al 2001, Am. J. Pathol. 159, 431-437) and increased PKB gamma activity has been observed in steroid independent breast and prostate cell lines (Nakatani et al 1999, J. Biol. Chem. 274, 21528-21532).

The PKB pathway also functions in the growth and survival of normal tissues and may be regulated during normal physiology to control cell and tissue function. Thus disorders associated with undesirable proliferation and survival of normal cells and tissues may also benefit therapeutically from treatment with a PKB inhibitor. Examples of such disorders are disorders of immune cells associated with prolonged expansion and survival of cell population leading to a prolonged or up regulated immune response. For example, T and B lymphocyte response to cognate antigens or growth factors such as interferon gamma activates the PI3K/PKB pathway and is responsible for maintaining the survival of the antigen specific lymphocyte clones during the immune response. Under conditions in which lymphocytes and other immune cells are responding to inappropriate self or foreign antigens, or in which other abnormalities lead to prolonged activation, the PKB pathway contributes an important survival signal preventing the normal mechanisms by which the immune response is terminated via apoptosis of the activated cell population. There is a considerable amount of evidence demonstrating the expansion of lymphocyte populations responding to self antigens in autoimmune conditions such as multiple sclerosis and arthritis. Expansion of lymphocyte populations responding inappropriately to foreign antigens is a feature of another set of conditions such as allergic responses and asthma. In summary inhibition of PKB could provide a beneficial treatment for immune disorders.

Other examples of inappropriate expansion, growth, proliferation, hyperplasia and survival of normal cells in which PKB may play a role include but are not limited to atherosclerosis, cardiac myopathy and glomerulonephritis.

In addition to the role in cell growth and survival, the PKB pathway functions in the control of glucose metabolism by insulin. Available evidence from mice deficient in the alpha and beta isoforms of PKB suggests that this action is mediated by the beta isoform primarily. As a consequence, modulators of PKB activity may also find utility in diseases in which there is a dysfunction of glucose metabolism and energy storage such as diabetes, metabolic disease and obesity.

Cyclic AMP-dependent protein kinase (PKA) is a serine/threonine protein kinase that phosphorylates a wide range of substrates and is involved in the regulation of many cellular processes including cell growth, cell differentiation, ion-channel conductivity, gene transcription and synaptic release of neurotransmitters. In its inactive form, the PKA holoenzyme is a tetramer comprising two regulatory subunits and two catalytic subunits.

PKA acts as a link between G-protein mediated signal transduction events and the cellular processes that they regulate. Binding of a hormone ligand such as glucagon to a transmembrane receptor activates a receptor-coupled G-protein (GTP-binding and hydrolyzing protein). Upon activation, the alpha subunit of the G protein dissociates and binds to and activates adenylate cyclase, which in turn converts ATP to cyclic-AMP (cAMP). The cAMP thus produced then binds to the regulatory subunits of PKA leading to dissociation of the associated catalytic subunits. The catalytic subunits of PKA, which are inactive when associated with the regulatory sub-units, become active upon dissociation and take part in the phosphorylation of other regulatory proteins.

For example, the catalytic sub-unit of PKA phosphorylates the kinase Phosphorylase Kinase which is involved in the phosphorylation of Phosphorylase, the enzyme responsible for breaking down glycogen to release glucose. PKA is also involved in the regulation of glucose levels by phosphorylating and deactivating glycogen synthase. Thus, modulators of PKA activity (which modulators may increase or decrease PKA activity) may be useful in the treatment or management of diseases in which there is a dysfunction of glucose metabolism and energy storage such as diabetes, metabolic disease and obesity.

PKA has also been established as an acute inhibitor of T cell activation. Anndahl et al, have investigated the possible role of PKA type I in HIV-induced T cell dysfunction on the basis that T cells from HIV-infected patients have increased levels of cAMP and are more sensitive to inhibition by cAMP analogues than are normal T cells. From their studies, they concluded that increased activation of PKA type I may contribute to progressive T cell dysfunction in HIV infection and that PKA type I may therefore be a potential target for immunomodulating therapy.—Aandahl, E. M., Aukrust, P., Skålhegg, B. S., Müller, F., Frøland, S. S., Hansson, V., Taskén, K. Protein kinase A type I antagonist restores immune responses of T cells from HIV-infected patients. FASEB J. 12, 855-862 (1998).

It has also been recognised that mutations in the regulatory sub-unit of PKA can lead to hyperactivation in endocrine tissue.

Because of the diversity and importance of PKA as a messenger in cell regulation, abnormal responses of cAMP can lead to a variety of human diseases derived from this, such as irregular cell growth and proliferation (Stratakis, C. A.; Cho-Chung, Y. S.; Protein Kinase A and human diseases. Trends Endrocri. Metab. 2002, 13, 50-52). Over-expression of PKA has been observed in a variety of human cancer cells including those from ovarian, breast and colon patients. Inhibition of PKA would therefore be an approach to treatment of cancer (Li, Q.; Zhu, G-D.; Current Topics in Medicinal Chemistry, 2002, 2, 939-971).

For a review of the role of PKA in human disease, see for example, Protein Kinase A and Human Disease, Edited by Constantine A. Stratakis, Annals of the New York Academy of Sciences, Volume 968, 2002, ISBN 1-57331-412-9.

Several classes of compounds have been disclosed as having PKA and PKB inhibitory activity.

WO 99/65909 (Pfizer) discloses a class of pyrrole[2,3-d]pyrimidine compounds having protein tyrosine kinase activity and which are of potential use as immunosuppressant agents.

WO 2004/074287 (AstraZeneca) discloses piperazinyl-pyridyl amides for use in treating autoimmune diseases such as arthritis. The piperazine group in the compounds can be linked to a purine group.

WO02/18348 (F. Hoffman La Roche) discloses a class of amino-quinazoline derivatives as alpha-1 adrenergic antagonists. A method for preparing the amino-quinazoline compounds involves the use of a gem-disubstituted cyclic amine such as piperidine in which one of the gem substituents is an aminomethyl group.

WO03/088908 (Bristol Myers Squibb) discloses N-heteroaryl-4,4-disubstituted piperidines as potassium channel inhibitors.

WO01/07050 (Schering) discloses substituted piperidines as nociceptin receptor ORL-1 agonists for use in treating cough.

US 2003/0139427 (OSI) discloses pyrrolidine- and piperidine-substituted purines and purine analogues having adenosine receptor binding activity.

WO 2004/043380 (Harvard College et al.) discloses technetium and rhenium labelled imaging agents containing disubstituted piperidine metal ion-chelating ligands.

WO 97/38665 (Merck) discloses gem-disubstituted piperidine derivatives having farnesyl transferase inhibitory activity.

EP 1568699 (Eisai) discloses 1,3-dihydroimidazole fused ring compounds having DPPIV-inhibiting activity. The compounds are described as having a range of potential uses including the treatment of cancer.

US 2003/0073708 and US 2003/045536 (both in the name of Castelhano et al), WO 02/057267 (OSI Pharmaceuticals) and WO 99/62518 (Cadus Pharmaceutical Corporation) each disclose a class of 4-aminodeazapurines in which the 4-amino group can form part of a cyclic amine such as azetidine, pyrrolidine and piperidine, The compounds are described as having adenosine receptor antagonist activity.

U.S. Pat. No. 6,162,804 (Merck) discloses a class of benzimidazole and aza-benzimidazole compounds that have tyrosine kinase inhibitor activity.

WO 2005/061463 (Astex) discloses pyrazole compounds having PKB and PKA inhibiting activity.

### SUMMARY OF THE INVENTION

The invention provides compounds that have protein kinase B (PKB) and/or protein kinase A (PKA) inhibiting or modulating activity, and which it is envisaged will be useful in preventing or treating disease states or conditions mediated by PKB and/or PKA.

Accordingly, in one aspect, the invention provides a compound of the formula (I):

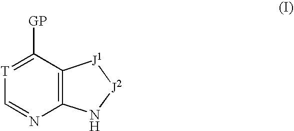

or salts, solvates, tautomers or N-oxides thereof, wherein

### (1) GP is a Group GP1:

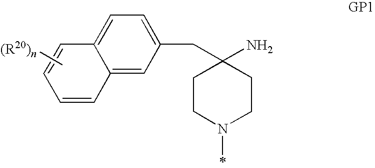

wherein the point of attachment to the bicyclic group is denoted by the asterisk;
R²⁰ is selected from fluorine, chlorine, C₁₋₄ alkoxy, trifluoromethyl, trifluoromethoxy, difluoromethoxy and C₁₋₄ alkyl; n is 0, 1 or 2; T is N; and J¹-J² is CH═CH; or

### (2) GP is a Group GP2:

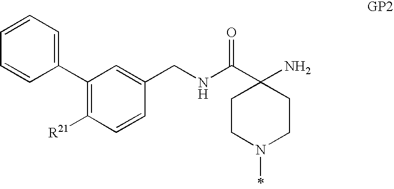

wherein the point of attachment to the bicyclic group is denoted by the asterisk;
R²¹ is selected from fluorine, chlorine, methoxy, trifluoromethyl, trifluoromethoxy, difluoromethoxy and methyl; T is N; and J¹-J² is CH═CH; or

### (2A) GP is a Group GP2A:

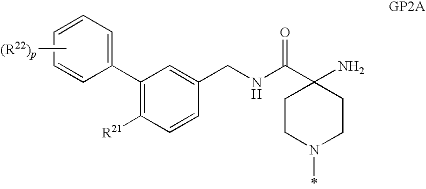

wherein the point of attachment to the bicyclic group is denoted by the asterisk;
R²¹ is selected from fluorine, chlorine, methoxy, trifluoromethyl, trifluoromethoxy, difluoromethoxy and methyl; R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH; or

### (3A) GP is a Group GP3A:

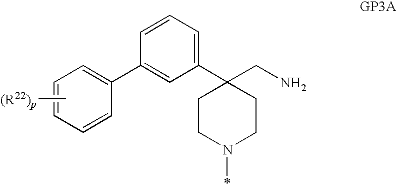

wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH; or

### (3B) GP is a Group GP3B:

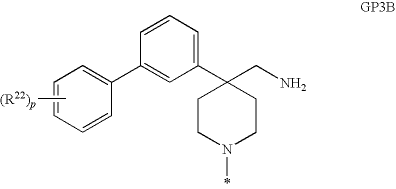

wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH, CH═N or CH₂CO; or

### (3C) GP is a Group GP3C:

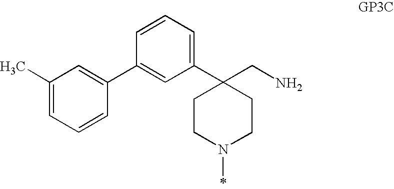

wherein the point of attachment to the bicyclic group is denoted by the asterisk;

### T is CH; and J¹-J² is CH₂CO; or

### (4) GP is a Group GP4:

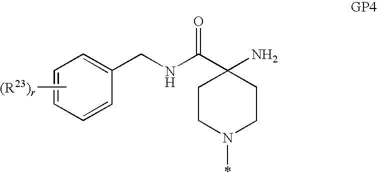

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); or

### (5) GP is a Group GP5:

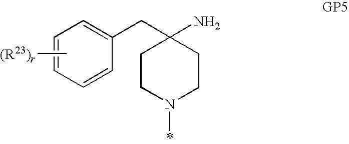

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); or

### (6) GP is a Group GP6:

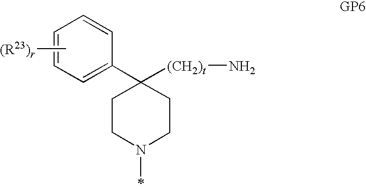

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2; t is 0 or 1; T is N; and J¹-J² is HN—C(O); provided that when r is 2, no more than 1 substituent R²³ can be an optionally substituted phenyl group; and provided also that when t is 1, r is 1 and R²³ is other than a 4-chloro substituent; or

### (7) GP is a Group GP7:

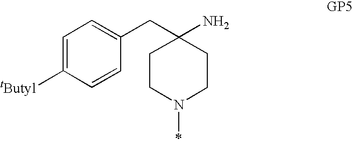

wherein the point of attachment to the bicyclic group is denoted by the asterisk;

### T is N; and J¹-J² is CH═N; or

### (8) GP is a Group GP8:

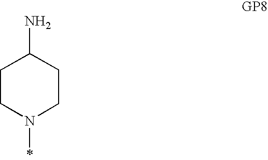

wherein the point of attachment to the bicyclic group is denoted by the asterisk;

### T is CH; and J¹-J² is CH₂—C(O); or

### (9) GP is a Group GP9:

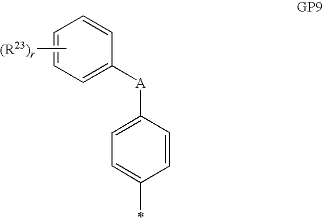

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; t is 0 or 1; T is N; and J¹-J² is HN—C(O); and A is selected from:
(i)

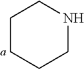

wherein the letter “a” denotes the point of attachment to the neighbouring benzene rings;
(ii) CH—CH₂—NHCH₃;
(iii) CH—CH₂—CH₂—NH₂; and
(iv) C(OH)—CH₂—CH₂—NH₂.

### (10) GP is a Group GP10:

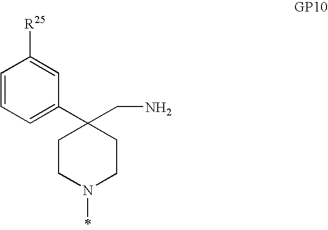

wherein the point of attachment to the bicyclic group is denoted by the asterisk;
R²⁵ is selected from hydrogen, fluorine; chlorine; C₁₋₄ alkoxy; trifluoromethyl; trifluoromethoxy; difluoromethoxy; and C₁₋₄ alkyl; T is CH or N; and J¹-J² is CH₂CO or CH═N; or

### (11) GP is a Group GP11:

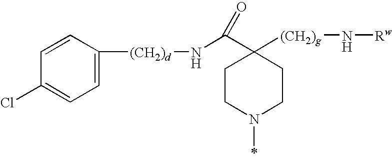

wherein (a) g is 0; d is 1; R^{w} is hydrogen or methyl; T is N; J¹-J² is N═CH, CH₂CO or CH═N; or (b) g is 1; d is 0 or 1; R^{w} is hydrogen; T is N; and J¹-J² is CH═CH; or

### (12) GP is a Group GP12:

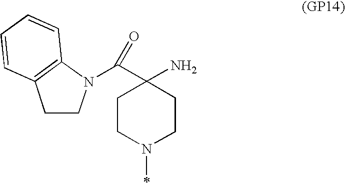

wherein T is N and J¹-J² is CH═CH; or

### (13) GP is a Group GP13:

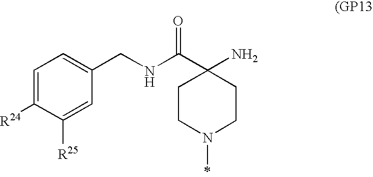

wherein T is N and J¹-J² is CH═CH; and (a) R²⁴ is methoxy and R²⁵ is hydrogen or chlorine; or (b) R²⁴ is methanesulphonyl or cyano and R²⁵ is hydrogen; or

### (14) GP is a Group GP14:

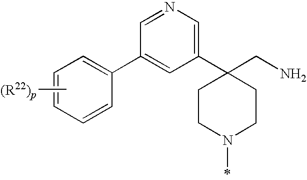

R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH.

In another aspect, the invention provides a compound of the formula (IA):

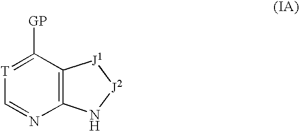

or salts, solvates, tautomers or N-oxides thereof, wherein

### (1) GP is a Group GP1:

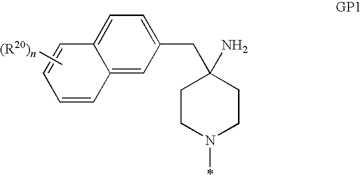

wherein the point of attachment to the bicyclic group is denoted by the asterisk;
R²⁰ is selected from fluorine, chlorine, C₁₋₄ alkoxy, trifluoromethyl, trifluoromethoxy, difluoromethoxy and C₁₋₄ alkyl; n is 0, 1 or 2; T is N; and J¹-J² is CH═CH; or

### (2) GP is a Group GP2:

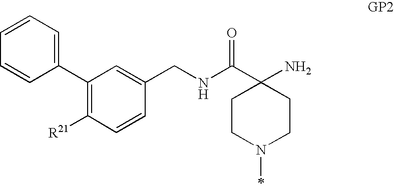

wherein the point of attachment to the bicyclic group is denoted by the asterisk;
R²¹ is selected from fluorine, chlorine, methoxy, trifluoromethyl, trifluoromethoxy, difluoromethoxy and methyl; T is N; and J¹-J² is CH═CH; or

### (3) GP is a Group GP3:

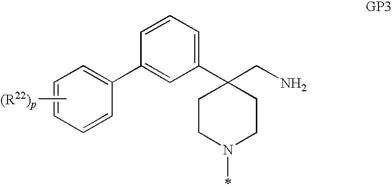

wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH; or

### (4) GP is a Group GP4:

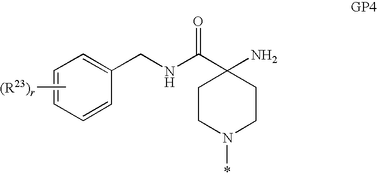

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); or

### (5) GP is a Group GP5:

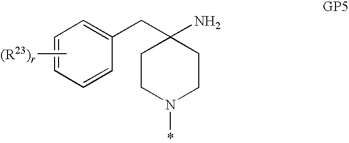

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); or

### (6) GP is a Group GP6:

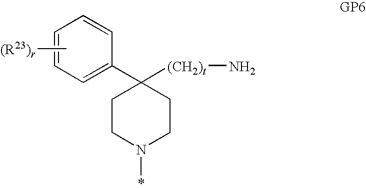

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2; t is 0 or 1; T is N; and J¹-J² is HN—C(O); provided that when r is 2, no more than 1 substituent R²³ can be an optionally substituted phenyl group; and provided also that when t is 1, r is 1 and R²³ is other than a 4-chloro substituent; or

### (7) GP is a Group GP7:

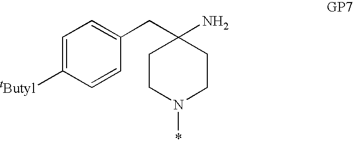

wherein the point of attachment to the bicyclic group is denoted by the asterisk;

### T is N; and J¹-J² is CH═N; or

### (8) GP is a Group GP8:

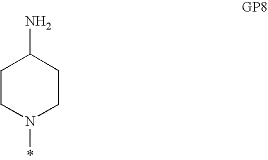

wherein the point of attachment to the bicyclic group is denoted by the asterisk;

### T is CH; and J¹-J² is CH₂—C(O); or

### (9) GP is a Group GP9:

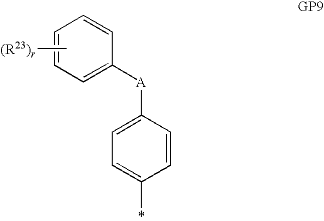

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); and A is selected from:
(i)

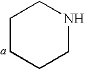

wherein the letter “a” denotes the point of attachment to the neighbouring benzene rings;
(ii) CH—CH₂—NHCH₃;
(iii) CH—CH₂—CH₂—NH₂; and
(iv) C(OH)—CH₂—CH₂—NH₂.

The invention also provides:
A compound of the formula (I) or any sub-group thereof as defined herein for use in the prophylaxis or treatment of a disease state or condition mediated by protein kinase B.
The use of a compound of formula (I) or any sub-group thereof as defined herein for the manufacture of a medicament for the prophylaxis or treatment of a disease state or condition mediated by protein kinase B.
A method for the prophylaxis or treatment of a disease state or condition mediated by protein kinase B, which method comprises administering to a subject in need thereof a compound of the formula (I) or any sub-group thereof as defined herein.
A compound of the formula (I) or any sub-group thereof as defined herein for use in treating a disease or condition comprising or arising from abnormal cell growth or abnormally arrested cell death in a mammal.
The use of a compound of formula (I) or any sub-group thereof as defined herein for the manufacture of a medicament for treating a disease or condition comprising or arising from abnormal cell growth or abnormally arrested cell death in a mammal.
A method for treating a disease or condition comprising or arising from abnormal cell growth in a mammal, which method comprises administering to the mammal a compound of the formula (I) or any sub-group thereof as defined herein in an amount effective in inhibiting abnormal cell growth or abnormally arrested cell death.
A method for alleviating or reducing the incidence of a disease or condition comprising or arising from abnormal cell growth or abnormally arrested cell death in a mammal, which method comprises administering to the mammal a compound of the formula (I) or any sub-group thereof as defined herein in an amount effective in inhibiting abnormal cell growth.
A method for treating a disease or condition comprising or arising from abnormal cell growth or abnormally arrested cell death in a mammal, the method comprising administering to the mammal a compound of the formula (I) or any sub-group thereof as defined herein in an amount effective to inhibit protein kinase B activity.
A compound of the formula (I) or any sub-group thereof as defined herein for use in inhibiting protein kinase B.
A method of inhibiting protein kinase B, which method comprises contacting the kinase with a kinase-inhibiting compound of the formula (I) or any sub-group thereof as defined herein.
A compound of the formula (I) or any sub-group thereof as defined herein for use in modulating a cellular process (for example cell division) by inhibiting the activity of a protein kinase B and/or protein kinase A.
The use of a compound of formula (I) or any sub-group thereof as defined herein for the manufacture of a medicament for modulating a cellular process (for example cell division) by inhibiting the activity of a protein kinase B and/or protein kinase A.
A method of modulating a cellular process (for example cell division) by inhibiting the activity of a protein kinase B and/or protein kinase A using a compound of the formula (I) or any sub-group thereof as defined herein.
A compound of the formula (I) or any sub-group or embodiment thereof as defined herein for use in the prophylaxis or treatment of a disease state or condition mediated by protein kinase A.
The use of a compound of formula (I) or any sub-group or embodiment thereof as defined herein for the manufacture of a medicament for the prophylaxis or treatment of a disease state or condition mediated by protein kinase A.
A method for the prophylaxis or treatment of a disease state or condition mediated by protein kinase A, which method comprises administering to a subject in need thereof a compound of the formula (I) or any sub-group or embodiment thereof as defined herein.
A method for treating a disease or condition comprising or arising from abnormal cell growth or abnormally arrested cell death in a mammal, the method comprising administering to the mammal a compound of the formula (I) or any sub-group or embodiment thereof as defined herein in an amount effective to inhibit protein kinase A activity.
A compound of the formula (I) or any sub-group or embodiment thereof as defined herein for inhibiting protein kinase A.
A method of inhibiting protein kinase A, which method comprises contacting the kinase with a kinase-inhibiting compound of the formula (I) or any sub-group or embodiment thereof as defined herein.
A method of modulating a cellular process (for example cell division) by inhibiting the activity of a protein kinase A using a compound of the formula (I) or any sub-group or embodiment thereof as defined herein.
The use of a compound of the formula (I) or any sub-group thereof as defined herein for the manufacture of a medicament for the prophylaxis or treatment of a disease state or condition arising from abnormal cell growth or abnormally arrested cell death.
A pharmaceutical composition comprising a novel compound of the formula (I) or any sub-group thereof as defined herein and a pharmaceutically acceptable carrier.
A compound of the formula (I) or any sub-group thereof as defined herein for use in medicine.
The use of a compound of the formula (I) or any sub-group thereof as defined herein for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I) or any sub-group thereof as defined herein.
A method for alleviating or reducing the incidence of a disease state or condition disclosed herein, which method comprises administering to a patient (e.g. a patient in need thereof) a compound (e.g. a therapeutically effective amount) of the formula (I) or any sub-group thereof as defined herein.
A method for the diagnosis and treatment of a disease state or condition mediated by protein kinase B, which method comprises (i) screening a patient to determine whether a disease or condition from which the patient is or may be suffering is one which would be susceptible to treatment with a compound having activity against protein kinase B; and (ii) where it is indicated that the disease or condition from which the patient is thus susceptible, thereafter administering to the patient a compound of the formula (I) or any sub-group thereof as defined herein.
The use of a compound of the formula (I) or any sub-group thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase B.
A compound of the formula (I) or any sub-group thereof as defined herein for use in the treatment or prophylaxis of a disease state or condition in a patient who has been screened and has been determined as suffering from, or being at risk of suffering from, a disease or condition which would be susceptible to treatment with a compound having activity against protein kinase B.
A method for the diagnosis and treatment of a disease state or condition mediated by protein kinase A, which method comprises (i) screening a patient to determine whether a disease or condition from which the patient is or may be suffering is one which would be susceptible to treatment with a compound having activity against protein kinase A; and (ii) where it is indicated that the disease or condition from which the patient is thus susceptible, thereafter administering to the patient a compound of the formula (I) or any sub-group or embodiment thereof as defined herein.
A compound of the formula (I) or any sub-group or embodiment thereof as defined herein for use in the treatment or prophylaxis of a disease state or condition in a patient who has been screened and has been determined as suffering from, or being at risk of suffering from, a disease or condition which would be susceptible to treatment with a compound having activity against protein kinase A.
The use of a compound of the formula (I) or any sub-group or embodiment thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase A.
A compound of the formula (I) or any sub-group or embodiment thereof as defined herein for use as a modulator (e.g. inhibitor) of protein kinase B and/or protein kinase A.
The use of a compound of the formula (I) or any sub-group or embodiment thereof as defined herein for the manufacture of a medicament for modulating (e.g. inhibiting) protein kinase B and/or protein kinase A.
A method of modulating (e.g. inhibiting) protein kinase B and/or protein kinase A; which method comprises bringing the protein kinase B and/or protein kinase A (e.g. in a cellular environment—for example in vivo) into contact with a compound of the formula (I) or any sub-group or embodiment thereof as defined herein.

### GENERAL PREFERENCES AND DEFINITIONS

Any references to Formula (I) herein shall be taken also to refer to any sub-group of compounds within formula (I), or any embodiment or example thereof, unless the context requires otherwise.

As used herein, the term “modulation”, as applied to the activity of a kinase, is intended to define a change in the level of biological activity of the protein kinase. Thus, modulation encompasses physiological changes which effect an increase or decrease in the relevant protein kinase activity. In the latter case, the modulation may be described as “inhibition”. The modulation may arise directly or indirectly, and may be mediated by any mechanism and at any physiological level, including for example at the level of gene expression (including for example transcription, translation and/or post-translational modification), at the level of expression of genes encoding regulatory elements which act directly or indirectly on the levels of kinase activity. Thus, modulation may imply elevated/suppressed expression or over- or under-expression of a kinase, including gene amplification (i.e. multiple gene copies) and/or increased or decreased expression by a transcriptional effect, as well as hyper- (or hypo-)activity and (de)activation of the protein kinase(s) (including (de)activation) by mutation(s). The terms “modulated”, “modulating” and “modulate” are to be interpreted accordingly.

As used herein, the term “mediated”, as used e.g. in conjunction with a kinase as described herein (and applied for example to various physiological processes, diseases, states, conditions, therapies, treatments or interventions) is intended to operate limitatively so that the various processes, diseases, states, conditions, treatments and interventions to which the term is applied are those in which the kinase plays a biological role. In cases where the term is applied to a disease, state or condition, the biological role played by a kinase may be direct or indirect and may be necessary and/or sufficient for the manifestation of the symptoms of the disease, state or condition (or its aetiology or progression). Thus, kinase activity (and in particular aberrant levels of kinase activity, e.g. kinase over-expression) need not necessarily be the proximal cause of the disease, state or condition: rather, it is contemplated that the kinase mediated diseases, states or conditions include those having multifactorial aetiologies and complex progressions in which the kinase in question is only partially involved. In cases where the term is applied to treatment, prophylaxis or intervention, the role played by the kinase may be direct or indirect and may be necessary and/or sufficient for the operation of the treatment, prophylaxis or outcome of the intervention. Thus, a disease state or condition mediated by a kinase includes the development of resistance to any particular cancer drug or treatment.

In this specification, references to “the bicyclic group”, when used in regard to the point of attachment of the group E shall, unless the context indicates otherwise, be taken to refer to the group:

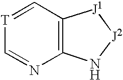

In formula (I) above and in the embodiments and sub-groups set out below, the term C₁₋₄ alkyl embraces methyl, ethyl, n-propyl, i-propyl, n-butyl, sec-butyl and tert-butyl.

Similarly, the term C₁₋₄ alkoxy embraces methoxy, ethoxy, n-propyloxy, i-propyloxy, n-butyloxy, sec-butyloxy and tert-butyloxy.

### GP1

In one embodiment of the invention, the moiety GP is a group GP1:

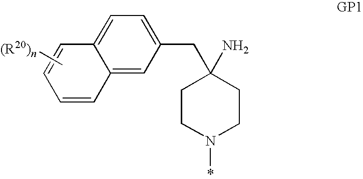

wherein the point of attachment to the bicyclic group is denoted by the asterisk;
R²⁰ is selected from fluorine, chlorine, C₁₋₄ alkoxy, trifluoromethyl, trifluoromethoxy, difluoromethoxy and C₁₋₄ alkyl; n is 0, 1 or 2; T is N; and J¹-J² is CH═CH.

Within this embodiment, particular compounds are those wherein n is 0 and those wherein n is 1 and R²⁰ is selected from fluorine and chlorine.

### GP2

In another embodiment of the invention, the moiety GP is a group GP2:

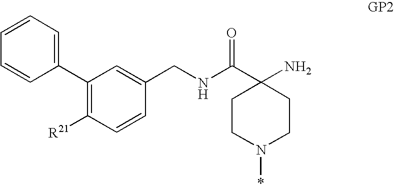

wherein the point of attachment to the bicyclic group is denoted by the asterisk;
R²¹ is selected from fluorine, chlorine, methoxy, trifluoromethyl, trifluoromethoxy, difluoromethoxy and methyl; T is N; and J¹-J² is CH═CH.

Particular groups R²¹ are fluorine and chlorine, with chlorine being a more particular example.

### GP2A

In another embodiment of the invention, the moiety GP is a group GP2A:

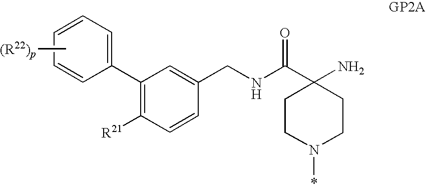

wherein the point of attachment to the bicyclic group is denoted by the asterisk;
R²¹ is selected from fluorine, chlorine, methoxy, trifluoromethyl, trifluoromethoxy, difluoromethoxy and methyl; R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH.

Preferably R²¹ is selected from methyl, fluorine and chlorine, and most preferably R²¹ is chlorine.

Preferably p is 0 or 1.

### GP3

In a further embodiment of the invention, the moiety GP is a group GP3:

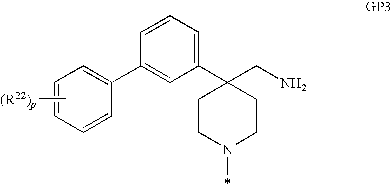

wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH.

Within this embodiment, particular compounds are those wherein p is 0 or 1, and more particularly p is 0. When p is other than 0 (e.g. p is 1), particular examples of R²² are fluorine and chlorine. When p is 1, a further particular example of R²² is methoxy, and more particularly para-methoxy.

### GP3A

In a further embodiment of the invention, the moiety GP is a group GP3A:

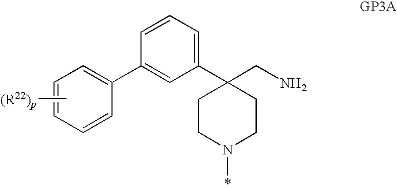

wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH, CH═N or CH₂CO.

In embodiment GP3A, p can be 0, 1 or 2. In one sub-group of compounds, p is 1 or 2.

The substituents R²² can be located at any of the ortho, meta and para positions around the phenyl ring.

Thus, the phenyl ring may be 2-monosubstituted, 3-monosubstituted, 4-monosubstituted, 2,3-disubstituted, 2,4-disubstituted, 2,5-disubstituted, 3,5-disubstituted or 2,6-disubstituted.

More particularly, the phenyl ring may be 2-monosubstituted, 3-monosubstituted, 4-monosubstituted, 2,3-disubstituted or 2,5-disubstituted.

Preferred substituents include methyl, methoxy, fluorine, chlorine and trifluoromethyl.

More preferred substituents include methyl, methoxy, fluorine and trifluoromethyl.

### GP3B

In a further embodiment of the invention, the moiety GP is a group GP3B:

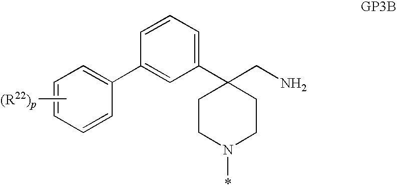

wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH, CH═N or CH₂CO.

Within this embodiment, in one sub-group of compounds, p is 0 or 1. Where p is 1, R²² may be selected from fluorine, chlorine, methoxy, trifluoromethyl, trifluoromethoxy, difluoromethoxy and methyl, and more particularly may be selected from methyl and methoxy.

### GP3C

In a further embodiment of the invention, the moiety GP is a group GP3C:

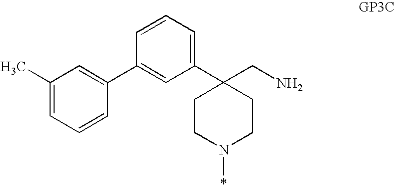

wherein the point of attachment to the bicyclic group is denoted by the asterisk;

### T is CH; and J¹-J² is CH₂CO.

### GP4

In another embodiment, the moiety GP is a group GP4:

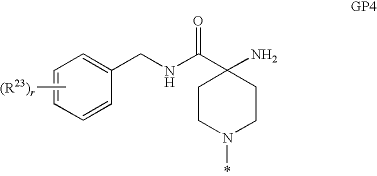

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O).

Within this embodiment, r is typically 1 or 2.

In one sub-group of compounds, r is 1.

When r is 1, the substituent group R²³ can be located at the 2-position, 3-position or 4-position of the phenyl ring.

In one particular sub-group, the substituent group R²³ is located at the 2-position of the phenyl ring.

In another particular sub-group, the substituent group R²³ is located at the 3-position of the phenyl ring.

In a further particular sub-group, the substituent group R²³ is located at the 4-position of the phenyl ring.

In each of the foregoing sub-groups, particular examples of R²³ are fluorine, chlorine and methoxy. In one preferred embodiment, r is 1 and R²³ is 4-choro.

In another sub-group of compounds, r is 2.

When r is 2, the substituents R²³ can be located at the 2- & 4-positions, the 2- & 3-positions or the 3- & 4-positions. In one sub-group of compounds, substituents R²³ are located at the 2- & 4-positions. Particular examples of R²³ when r is 2 are fluorine, chlorine and methoxy. In one preferred embodiment, r is 2 and the phenyl ring bearing the two substituents R²³ is 4-chloro-2-fluorophenyl.

### GP5

In another embodiment, the moiety GP is a group GP5:

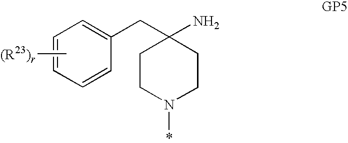

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O).

Within this embodiment, r is typically 1 or 2.

In one sub-group of compounds, r is 1.

When r is 1, the substituent group R²³ can be located at the 2-position, 3-position or 4-position of the phenyl ring.

In one particular sub-group, the substituent group R²³ is located at the 2-position of the phenyl ring.

In another particular sub-group, the substituent group R²³ is located at the 3-position of the phenyl ring.

In a further particular sub-group, the substituent group R²³ is located at the 4-position of the phenyl ring.

In each of the foregoing sub-groups, particular examples of R²³ are fluorine, chlorine, trifluoromethoxy, methyl, tert-butyl and methoxy. In one preferred embodiment, r is 1 and R²³ is 4-choro, 4-trifluoromethoxy or 4-tert-butyl.

In another sub-group of compounds, r is 2.

When r is 2, the substituents R²³ can be located at the 2- & 4-positions, the 2- & 3-positions or the 3- & 4-positions. In one sub-group of compounds, substituents R²³ are located at the 2- & 4-positions. Particular examples of R²³ when r is 2 are fluorine, chlorine and methoxy. In one preferred embodiment, r is 2 and the phenyl ring bearing the two substituents R²³ is 2,4-dichlorophenyl.

### GP6

In a further embodiment, the moiety GP is a group GP6:

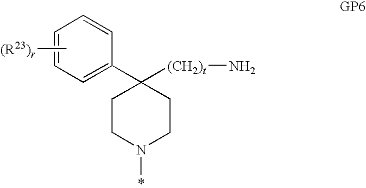

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2; t is 0 or 1; T is N; and J¹-J² is HN—C(O); provided that when r is 2, no more than 1 substituent R²³ can be an optionally substituted phenyl group; and provided also that when t is 1, r is 1 and R²³ is other than a 4-chloro substituent.

In one sub-group of compounds, t is 1.

Within this sub-group, particular compounds are those in which r is 1 and R²³ is selected from fluorine; 2-chloro; 3-chloro; methoxy; trifluoromethyl; trifluoromethoxy; difluoromethoxy; methyl; ethyl; isopropyl; tert-butyl; and phenyl optionally substituted by fluorine, chlorine, methoxy, trifluoromethyl, trifluoromethoxy, difluoromethoxy or methyl.

More particular compounds are those wherein r is 1 and R²³ is selected from fluorine; 2-chloro; 3-chloro; methoxy; trifluoromethyl; trifluoromethoxy; difluoromethoxy; methyl; ethyl; isopropyl; and tert-butyl.

In another sub-group of compounds, t is 0.

Within this sub-group, particular compounds are those in which r is 1 and R²³ is selected from fluorine; chlorine; methoxy; trifluoromethyl; trifluoromethoxy; difluoromethoxy; methyl; ethyl; isopropyl; tert-butyl; and phenyl optionally substituted by fluorine, chlorine, methoxy, trifluoromethyl, trifluoromethoxy, difluoromethoxy or methyl.

More particular compounds are those wherein r is 1 and R²³ is selected from fluorine; chlorine; methoxy; trifluoromethyl; trifluoromethoxy; difluoromethoxy; methyl; ethyl; isopropyl; and tert-butyl.

### GP7

In another embodiment, the moiety GP is a group GP7:

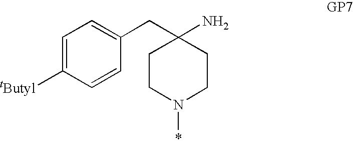

wherein the point of attachment to the bicyclic group is denoted by the asterisk;

### T is N; and J¹-J² is CH═N.

### GP8

In another embodiment, the moiety GP is a group GP8:

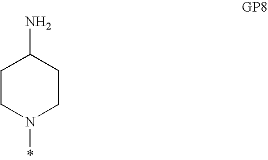

wherein the point of attachment to the bicyclic group is denoted by the asterisk; T is CH; and J¹-J² is CH₂—C(O).

### GP9

In another embodiment, the moiety GP is a group GP9:

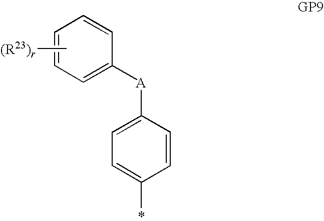

wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); and A is selected from:
(i)

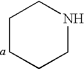

wherein the letter “a” denotes the point of attachment to the neighbouring benzene rings;
(ii) CH—CH₂—NHCH₃;
(iii) CH—CH₂—CH₂—NH₂; and
(iv) C(OH)—CH₂—CH₂—NH₂.

Within this embodiment, in one sub-group of compounds, A is:

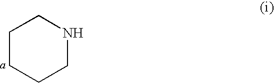

In another sub-group of compounds, A is (ii) CH—CH₂—NHCH₃.

In a further sub-group of compounds, A is (iii) CH—CH₂—CH₂—NH₂.

In a still further sub-group of compounds, A is (iv) C(OH)—CH₂—CH₂—NH₂.

In each of sub-groups (i) to (iv), r is typically 1 or 2.

When r is 1, the substituent group R²³ can be located at the 2-position, 3-position or 4-position of the phenyl ring.

For example, the substituent group R²³ can be located at the 2-position of the phenyl ring.

Alternatively, the substituent group R²³ can be located at the 3-position of the phenyl ring.

In a further alternative, the substituent group R²³ can be located at the 4-position of the phenyl ring.

In each of the foregoing sub-groups, examples and alternatives, particular examples of R²³ are fluorine, chlorine, trifluoromethoxy, methyl, tert-butyl and methoxy. In one preferred embodiment, r is 1 and R²³ is 4-choro.

In another sub-group of compounds, r is 2.

When r is 2, the substituents R²³ can be located at the 2- & 4-positions, the 2- & 3-positions or the 3- & 4-positions. In one sub-group of compounds, substituents R²³ are located at the 2- & 4-positions. Particular examples of R²³ when r is 2 are fluorine, chlorine and methoxy. In one preferred embodiment, r is 2 and the phenyl ring bearing the two substituents R²³ is 2,4-dichlorophenyl.

### GP10

In another embodiment, GP is a group GP10:

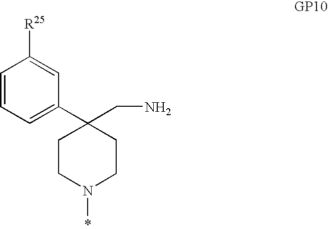

wherein the point of attachment to the bicyclic group is denoted by the asterisk;
R²⁵ is selected from hydrogen, fluorine; chlorine; C₁₋₄ alkoxy; trifluoromethyl; trifluoromethoxy; difluoromethoxy; and C₁₋₄ alkyl; T is CH or N; and J¹-J² is CH₂CO or CH═N.

More particularly, R²⁵ is selected from hydrogen, fluorine; chlorine; methoxy; trifluoromethyl; trifluoromethoxy; difluoromethoxy; and methyl.

Preferably, R²⁵ is selected from hydrogen and chlorine.

In one sub-group of compounds within GP10, J¹-J² is CH₂CO.

In another sub-group of compounds within GP10, J¹-J² is CH═N.

In each of the above two sub-groups, T can be CH.

Alternatively, in each of the above two sub-groups, T can be N.

### GP11

In another embodiment, GP is a group GP11:

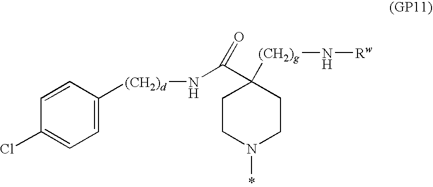

wherein (a) g is 0; d is 1; R^{w} is hydrogen or methyl; T is N; J¹-J² is N═CH, CH₂CO or CH═N; or (b) g is 1; d is 0 or 1; R^{w} is hydrogen; T is N; and J¹-J² is CH═CH.

In one sub-group of compounds, g is 0; d is 1; R^{w} is hydrogen or methyl; T is N; and J¹-J² is N═CH, CH₂CO or CH═N.

Within this sub-group, particular compounds are those wherein J¹-J² is N═CH or CH₂CO.

In another sub-group of compounds, g is 1; d is 0 or 1; R^{w} is hydrogen; T is N; and J¹-J² is CH═CH.

### GP12

In another embodiment, GP is a group GP12:

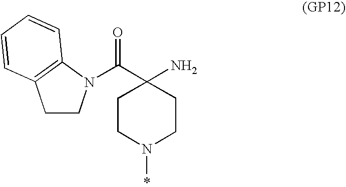

wherein T is N and J¹-J² is CH═CH.

### GP13

In another embodiment, GP is a group GP13:

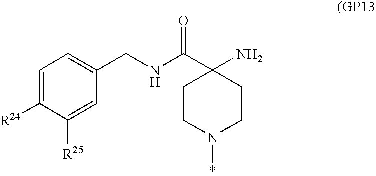

wherein T is N and J¹-J² is CH═CH; and (a) R²⁴ is methoxy and R²⁵ is hydrogen or chlorine; or (b) R²⁴ is methanesulphonyl or cyano and R²⁵ is hydrogen.

### GP14

In another embodiment, GP is a group GP14:

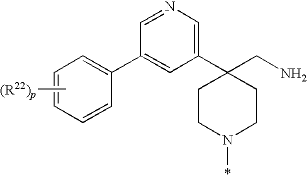

R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH.

More particularly, R²² is selected from fluorine, chlorine, methoxy, trifluoromethyl, trifluoromethoxy, difluoromethoxy and methyl, and p is 1.

Still more particularly, R²² is methyl.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples below.

### Salts, Solvates, Tautomers, Isomers, N-Oxides, Esters, Prodrugs and Isotopes

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to all sub-groups thereof unless the context indicates otherwise.

Salt forms may be selected and prepared according to methods described in Pharmaceutical Salts Properties, Selection, and Use, P. Heinrich Stahl (Editor), Camille G. Wermuth (Editor), ISBN: 3-90639-026-8, Hardcover, 388 pages, August 2002. For example, acid addition salts may be prepared by dissolving the free base in an organic solvent in which a given salt form is insoluble or poorly soluble and then adding the required acid in an appropriate solvent so that the salt precipitates out of solution.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(s)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (±)-L-lactic and (±)-DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

One particular group of acid addition salts includes salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids. Within this group of salts, a sub-set of salts consists of salts formed with hydrochloric acid or acetic acid.

Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

The compounds of the invention may exist as mono- or di-salts depending upon the pKa of the acid from which the salt is formed. In stronger acids, the basic pyrazole nitrogen, as well as the nitrogen atom in the group NR²R³, may take part in salt formation. For example, where the acid has a pKa of less than about 3 (e.g. an acid such as hydrochloric acid, sulphuric acid or trifluoroacetic acid), the compounds of the invention will typically form salts with 2 molar equivalents of the acid.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, when J¹-J² is N═CR⁶, the tautomeric forms A and B are possible for the bicyclic group.

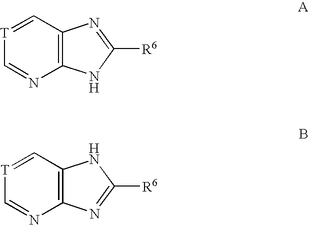

When J¹-J² is HN—CO, the tautomeric forms C, D and E are possible for the bicyclic group.

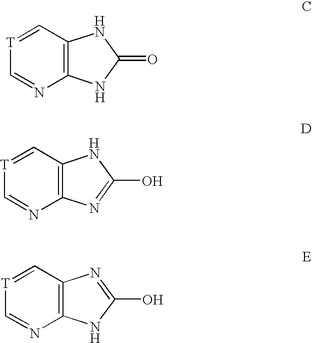

All such tautomers are embraced by formula (I).

Other examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

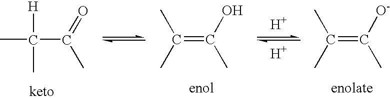

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic or scalemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluloyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters of the compounds of formula (I) bearing a hydroxyl group are also embraced by Formula (I). In one embodiment of the invention, formula (I) includes within its scope esters of compounds of the formula (I) bearing a hydroxyl group. In another embodiment of the invention, formula (I) does not include within its scope esters of compounds of the formula (I) bearing a hydroxyl group. Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the hydroxyl groups (—C(═O)OH) in the parent compound, with, where appropriate, prior protection of any other reactive groups present in the parent compound, followed by deprotection if required.

Examples of such metabolically labile esters include those of the formula—

C(═O)OR wherein R is:
C₁₋₇alkyl
(e.g., -Me, -Et, -nPr, -iPr, -nBu, -sBu, -iBu, -tBu);
C₁₋₇-aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy) carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in Antibody-directed Enzyme Prodrug Therapy (ADEPT), Gene-directed Enzyme Prodrug Therapy (GDEPT), Polymer-directed Enzyme Prodrug Therapy (PDEPT), Ligand-directed Enzyme Prodrug Therapy (LIDEPT), etc.). For example, the prodrug may be a sugar derivative or other glycoside conjugate, or may be an amino acid ester derivative.

### Methods for the Preparation of Compounds of the Formula (I)

In this section, references to compounds of the formula (I) include each of the sub-groups thereof as defined herein unless the context requires otherwise.

In a further aspect, the invention provides a process for the preparation of a compound of the formula (I) as defined herein.

Compounds of the formula (I) wherein GP is a group GP9:

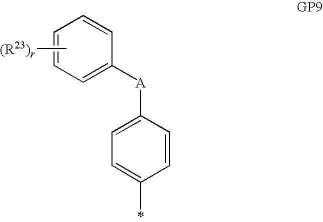

can be prepared by reaction of a compound of the formula (X) with a compound of the formula (XI) where (X) and (XI) may be suitably protected and wherein T, J¹, J², A, r and R²³ are as hereinbefore defined, one of the groups X and Y is chlorine, bromine or iodine or a trifluoromethanesulphonate (triflate) group, and the other one of the groups X and Y is a boronate residue, for example a boronate ester or boronic acid residue.

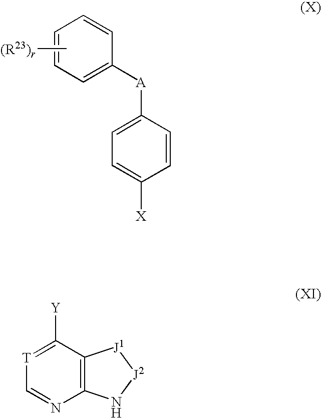

The reaction can be carried out under typical Suzuki coupling conditions in the presence of a palladium catalyst such as tetrakis(triphenylphosphine)palladium and a base (e.g. a carbonate such as potassium carbonate). The reaction may be carried out in a polar solvent, for example an aqueous solvent such as aqueous ethanol, or an ether such as dimethoxyethane, and the reaction mixture is typically subjected to heating, for example to a temperature of 80° C. or more, e.g. a temperature in excess of 100° C.

An illustrative synthetic route involving a Suzuki coupling step is shown in Scheme 1. In Scheme 1, the bromo compound (XII) is converted to a boronic acid (XIII) by reaction with an alkyl lithium such as butyl lithium and a borate ester (iPrO)₃B. The reaction is typically carried out in a dry polar solvent such as tetrahydrofuran at a reduced temperature (for example −78° C.).

The resulting boronic acid (XIII) is then reacted with the N-protected chloro compound (XIV) in the presence of bis(triphenylphosphine)palladium under the conditions described above. The protecting group P (which can be for example a tetrahydropyranyl (THP) group) is then removed by treatment with an acid such as hydrochloric acid to give the compound of the formula (I′).

In Scheme 1, the amino group in GP9 is typically protected with a suitable protecting group of which examples are set out below. One particular protecting group which may be used in the context of a Suzuki coupling for protecting an amino group is the tert-butoxycarbonyl group which can be introduced by reacting the amino group with di-tert-butylcarbonate in the presence of a base such as triethylamine. Removal of the protecting group is typically accomplished at the same time as removal of the protecting group P on the bicyclic group.

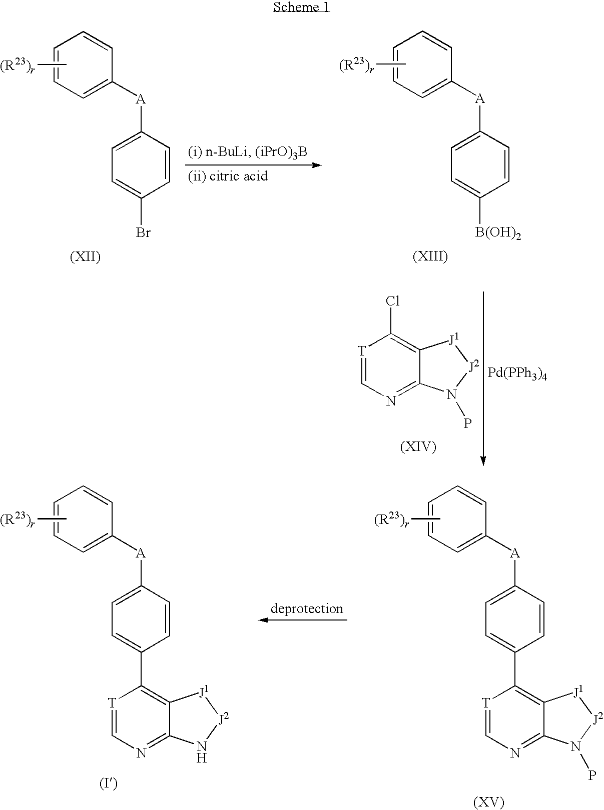

In the preparative procedure outlined above, the coupling of the benzene ring to the bicyclic group is accomplished by reacting a halo-purine (or deaza analogue thereof) or halo-aryl or heteroaryl compound with a boronate ester or boronic acid in the presence of a palladium catalyst and base. Many boronates suitable for use in preparing compounds of the invention are commercially available, for example from Boron Molecular Limited of Noble Park, Australia, or from Combi-Blocks Inc, of San Diego, USA. Where the boronates are not commercially available, they can be prepared by methods known in the art, for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid.

Compounds of the formula (I) wherein GP is a group GP1, GP2, GP2A, GP3, GP3A, GP3B, GP3C, GP4, GP5, GP6, GP7, GP8, GP10, GP11, GP12, GP13 and GP14, i.e. wherein a 4-disubstituted piperidine ring is linked to the bicyclic group by a nitrogen atom, can be prepared by the reaction of a compound of the formula (XVI), or a protected derivative thereof, where T is N and Hal is chlorine or fluorine (more usually chlorine), with a compound of the formula (XVII) or a protected derivative thereof, where R′ and R″ represent the residues of the group GP.

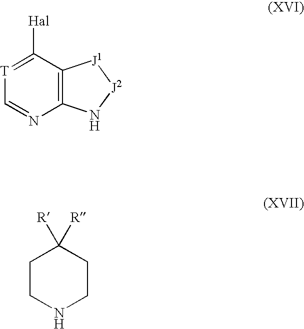

The reaction is typically carried out in a polar solvent such as an alcohol (e.g. ethanol, propanol or n-butanol) at an elevated temperature, for example a temperature in the region from 90° C. to 160° C., optionally in the presence of a non-interfering amine such as triethylamine. The reaction may be carried out in a sealed tube, particularly where the desired reaction temperature exceeds the boiling point of the solvent. When T is N, the reaction is typically carried out at a temperature in the range from about 100° C. to 130° C. but, when T is CH, higher temperatures may be required, for example up to about 160° C., and hence higher boiling solvents such as N-methylpyrrolidinone (NMP) or dimethylformamide may be used. In general, an excess of the nucleophilic amine will be used and/or an additional non-reacting base such as triethylamine will be included in the reaction mixture. Heating of the reaction mixture may be accomplished by normal means or by the use of a microwave heater.

In order to prepare compounds of the formula (I) wherein T is CH, the hydrogen atom of the group CH may be replaced by an activating group in order to facilitate nucleophilic displacement of the chlorine atom by the amine (XVII). The activating group is typically one which can be removed subsequent to the nucleophilic displacement reaction. One such activating group is an ester group such as ethoxycarbonyl or methoxycarbonyl which can be removed by hydrolysis and decarboxylation. Hydrolysis of the ethoxycarbonyl or methoxycarbonyl group to the carboxylic acid is typically carried out using an aqueous alkali such as sodium hydroxide, and the decarboxylation step is typically conducted by heating to an elevated temperature (e.g. 150° C. to 190° C.). As an alternative to employing an activating group, it is possible to use a compound of the formula (XVI) in which Hal is a fluorine atom and the nitrogen atom at the 1-position of the five membered ring is protected by a suitable protecting group (e.g. a triisopropylsilanyl group).

Compounds of the formula (XVI) are commercially available or can be prepared according to methods well known to the skilled person. For example, compounds of the formula (XVI) where T is N and J¹-J² is CH═N, can be prepared from the corresponding hydroxy compounds by reaction with a chlorinating agent such as POCl₃. Compounds of the formula (XVI) where J¹-J² is HN—C(O) can be prepared by the reaction of an ortho-diamino compound of the formula (XVIII) with carbonyl di-imidazole in the presence of a non-interfering base such as triethylamine.

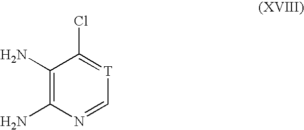

Compounds of the formula (XVI) where T is CH and J¹-J² is H₂C═CH₂) can be prepared from the corresponding N-oxide of the formula (XIX) by reaction with phosphorus oxychloride at an elevated temperature, for example the reflux temperature of POCl₃.

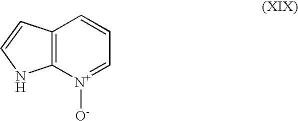

Intermediate compounds of the formula (XVII) wherein the R″ and R′ are an amino group and an optionally substituted benzyl or naphthylmethyl group respectively can be prepared by the sequence of reactions shown in Scheme 2. In Scheme 2, the moiety “P” is a protecting group and the group “Ar” is an optionally substituted phenyl or naphthyl group.

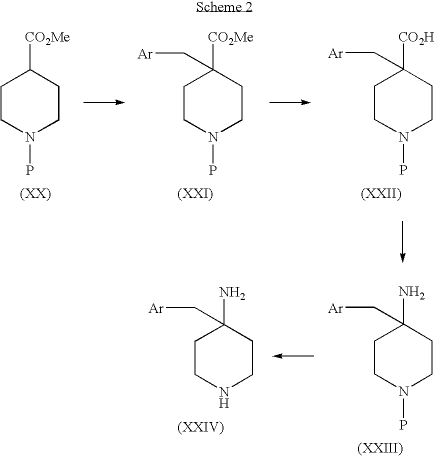

In Scheme 2,4-methoxycarbonyl-piperidine is first protected in standard fashion, for example by means of a t-butyloxycarbonyl (boc) group by reaction with di-tert-butylcarbonate in the presence of a non-interfering base to give the protected compound (XX). The protected piperidine carboxymethyl ester (XX) is then alkylated at the α-position relative to the carbonyl group of the ester by reacting with a strong base such as lithium diisopropylamide (LDA) and a compound of the formula ArCH₂-Hal where Hal is a halogen, preferably bromine. The ester (XXI) is then hydrolysed to the corresponding carboxylic acid (XXII) using an alkali metal hydroxide such as sodium hydroxide. The carboxylic acid (XXII) can be used to prepare a range of different amine intermediates which can, in turn, be converted into compounds of the formula (II). For example, as shown in Scheme 2, the carboxylic acid can be converted to the acid chloride (e.g. by treatment with oxalyl chloride and optionally a catalytic quantity of DMF, or by treatment of a salt of the acid with oxalyl chloride) and then reacted with sodium azide to form the acid azide (not shown). The acid azide can then be heated to bring about rearrangement in a Curtius reaction (see Advanced Organic Chemistry, 4^{th} edition, by Jerry March, John Wiley & sons, 1992, pages 1091-1092) to give compound (XXIII) in which the amino group is attached directly to the piperidine ring. The amine (XXIII) is then deprotected according to standard methods (e.g. using hydrochloric acid in the case of a Boc protecting group) and reacted with a compound of the formula (XIV) to give a compound of the formula (I).

Compounds of the formula ((XVII) in which R′ is a substituted phenyl group and R″ is a CH₂NH₂ group (i.e. as in compounds of the formula (I) GP is GP3) can be prepared using the sequence of steps shown in Scheme 3.

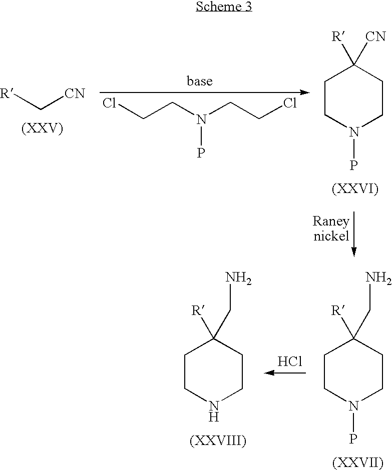

As shown in Scheme 3, the nitrile (XXV) in which R′ is a substituted phenyl group is reacted with a base and N-protected (P=protecting group) bis-(2-chloroethyl)amine to give the piperidine nitrile (XXVI) which can then be reduced to give the amine (XXVII) using Raney nickel and then deprotected (e.g. using HCl when the protecting group is acid labile) to give amine (XXVIII).

Compounds of the formula (I) in which R′ is a substituted benzyl or naphthylmethyl group and R″ is a NH₂ group can also be prepared by the reaction sequence shown in Scheme 4.

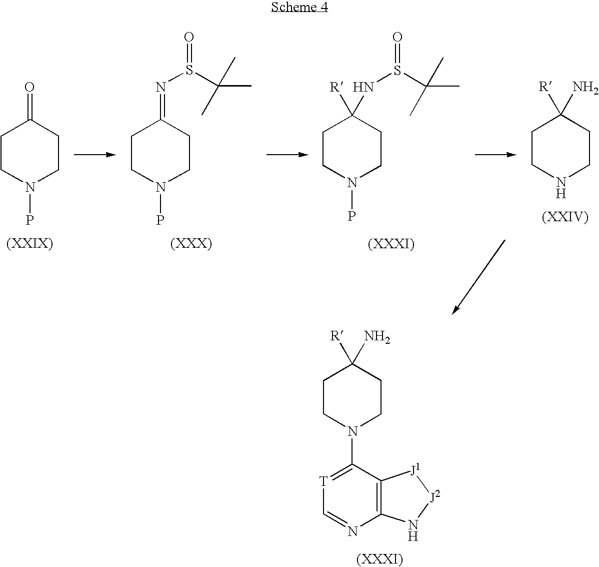

As shown in Scheme 4, a protected 4-piperidone (XXIX), in which P is a protecting group such as Boc, is reacted with tert-butylsulphinimide in the presence of titanium tetraethoxide in a dry polar solvent such as THF to give the sulphinimine (XXX). The reaction is typically carried out with heating, for example to the reflux temperature of the solvent. The sulphinimine (XXX) is then reacted with an organometallic reagent, for example a Grignard reagent such as a substituted benzylmagnesium bromide, suitable for introducing the moiety R′, to give the sulphinamide (XXXI). The tert-butylsulphinyl group can then be removed by hydrolysis in a hydrochloric acid/dioxane/methanol mixture to give the amine (XXIV). The amine (XXIV) can then be reacted with a chloro-heterocycle (XVI) under the conditions described above to give the product (XXXI.

Compounds of the formula (I) wherein GP is a group GP2 or GP4 containing an amide bond can be prepared from intermediates of the formula (XXXII) by reaction with intermediate (XVI) using the methods and conditions described above.

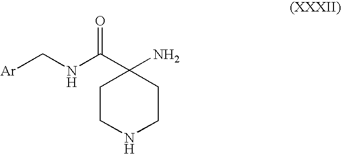

In formula (XXXII), Ar is a substituted phenyl group of the type present in GP2 and GP4.

The compounds of formula (XXXII) can be prepared by reacting together the appropriate carboxylic acid or activated derivative thereof (e.g. acid chloride) and the appropriate amine using the amide-forming conditions described above.

The formation of compounds of the formula (I) wherein GP is GP2, GP2A, GP4, GP10 (f=1), GP11 or GP13 is illustrated by the sequence of reactions set out in Scheme 5.

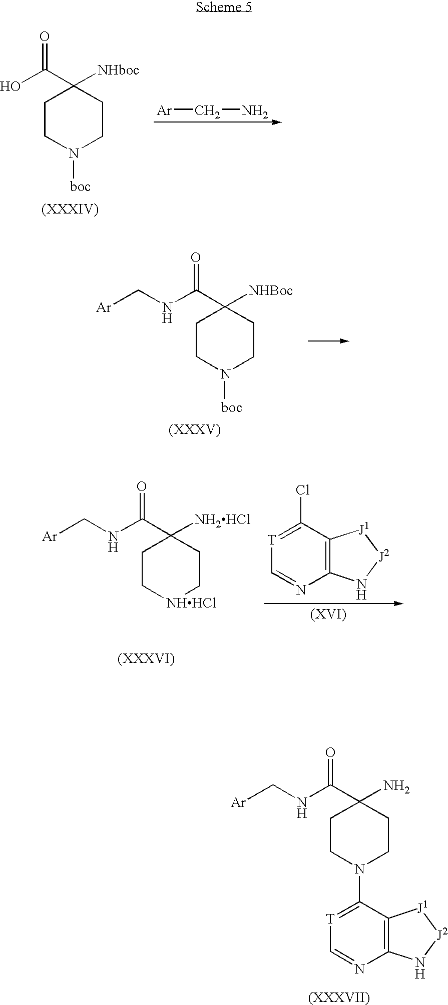

In Scheme 5, the boc-protecte or heteroarylamine ArCH₂—NH₂ using standard amide forming conditions. Thus, for example, the reaction is preferably carried out in the presence of a reagent of the type commonly used in the formation of peptide linkages. Examples of such reagents include 1,3-dicyclohexylcarbodiimide (DCC) (Sheehan et al, J. Amer. Chem. Soc. 1955, 77, 1067), 1-ethyl-3-(3′-dimethylaminopropyl)-carbodiimide (referred to herein either as EDC or EDAC) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

Compounds of the formula (I) wherein T is CH and J¹-J² is CH═N or CH═CH can be prepared according to the procedure illustrated in Scheme 6.

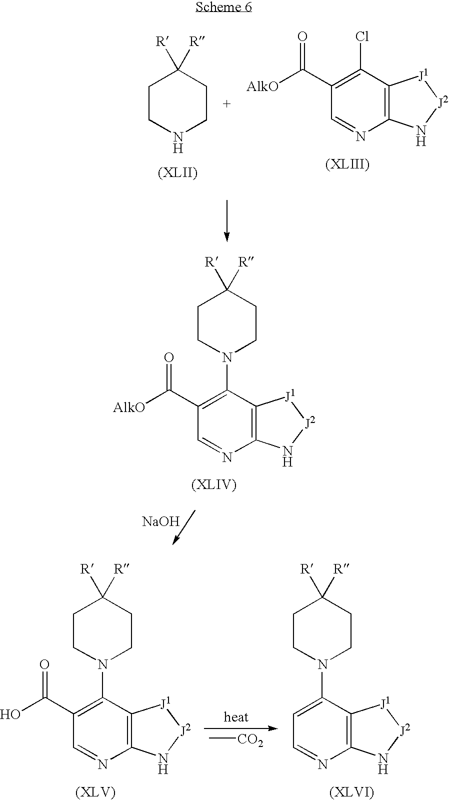

In the sequence of reactions shown in Scheme 7, the starting material is the chlorinated carboxy ester compound (XLIII) which can be prepared by methods generally analogous to methods described in J. Heterocycl. Chem. 1972, 235 and Bioorg. Med. Chem. Lett. 2003, 2405 followed by removal of any unwanted protecting groups where necessary. In formula (XLIII), AlkO is an alkoxy group, e.g. a C₁₋₃ alkoxy group such as methoxy or ethoxy (particularly ethoxy).

The substituted piperidine compound (XLII), suitably protected where necessary, is reacted with the chlorinated carboxy ester compound (XLIII), to give an ester intermediate of the formula (XLIV). The reaction may be carried out in a polar solvent such as a higher boiling alcohol (e.g. n-butanol) in the presence of a non-interfering base such as triethylamine at an elevated temperature (e.g. 90° C. to 130° C., more typically 100° C. to 120° C.). Heating can be effected by means of a microwave heater.

The carboxy ester group in the chlorinated carboxy ester compound (XLIII) functions as an activating group, rendering the chlorine atom more susceptible to nucleophilic displacement. Once the nucleophilic displacement reaction has taken place, the carboxy ester group has served its purpose and can be removed. Accordingly, hydrolysis of the ester intermediate (XLIV) to the carboxylic acid (XLV) is carried out using an aqueous alkali metal hydroxide such as potassium hydroxide or sodium hydroxide with heating where necessary. The carboxylic acid (XLV) is then decarboxylated to give the product (XLVI) by heating to an elevated temperature in excess of 100° C., for example a temperature in the range from about 120° C. to about 180° C.).

Once formed, many compounds of the formula (I) can be converted into other compounds of the formula (I) using standard functional group interconversions.

Examples of functional group interconversions and reagents and conditions for carrying out such conversions can be found in, for example, Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

### Protecting Groups

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2-(phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

### Isolation and Purification of the Compounds of the Invention

The compounds of the invention can be isolated and purified according to standard techniques well known to the person skilled in the art. One technique of particular usefulness in purifying the compounds is preparative liquid chromatography using mass spectrometry as a means of detecting the purified compounds emerging from the chromatography column.

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

### Chemical Intermediates

Many of the chemical intermediates described above are novel per se and such novel intermediates form a further aspect of the invention.

Examples of such intermediates include, but are not limited to, protected forms of compounds of the formula (I) and sub-groups thereof, such as protected forms of compounds of the formulae (I′), (XXXI), (XXXVII), and (XLVI), as well as compounds of the formulae (XLIV) and (XLV) and protected forms thereof.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing compounds of the formula (I) can be formulated in accordance with known techniques, see for example, Remington's Pharmaceutical Sciences, Mack Publishing Company, Easton, Pa., USA.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of formula (I), or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. A tablet can contain the solid solution blended with at least one disintegrant, a lubricant, a surfactant, and a glidant. The chewable tablet can contain the solid solution blended with a bulking agent, a lubricant, and if desired an additional sweetening agent (such as an artificial sweetener), and suitable flavours.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

### Protein Kinase Inhibitory Activity

The activity of the compounds of the invention as inhibitors of protein kinase A and protein kinase B can be measured using the assays set forth in the examples below and the level of activity exhibited by a given compound can be defined in terms of the IC50 value. Preferred compounds of the present invention are compounds having an IC₅₀ value of less than 1 μM, more preferably less than 0.1 μM, against protein kinase B.

Some of the compounds of the formula (I) are selective inhibitors of PKB relative to PKA, i.e. the IC₅₀ values against PKB are from 5 to 10 times lower, and more preferably greater than 10 times lower, than the IC₅₀ values against PKA.

### Therapeutic Uses

### Prevention or Treatment of Proliferative Disorders

The compounds of the formula (I) are inhibitors of protein kinase A and protein kinase B. As such, they are expected to be useful in providing a means of preventing the growth of or inducing apoptosis of neoplasias. It is therefore anticipated that the compounds will prove useful in treating or preventing proliferative disorders such as cancers. In particular tumours with deletions or inactivating mutations in PTEN or loss of PTEN expression or rearrangements in the (T-cell lytmphocyte) TCL-1 gene may be particularly sensitive to PKB inhibitors. Tumours which have other abnormalities leading to an upregulated PKB pathway signal may also be particularly sensitive to inhibitors of PKB. Examples of such abnormalities include but are not limited to overexpression of one or more PI3K subunits, over-expression of one or more PKB isoforms, or mutations in PI3K, PDK1, or PKB which lead to an increase in the basal activity of the enzyme in question, or upregulation or overexpression or mutational activation of a growth factor receptor such as a growth factor selected from the epidermal growth factor receptor (EGFR), fibroblast growth factor receptor (FGFR), platelet derived growth factor receptor (PDGFR), insulin-like growth factor 1 receptor (IGF-1R) and vascular endothelial growth factor receptor (VEGFR) families.

It is also envisaged that the compounds of the invention will be useful in treating other conditions which result from disorders in proliferation or survival such as viral infections, and neurodegenerative diseases for example. PKB plays an important role in maintaining the survival of immune cells during an immune response and therefore PKB inhibitors could be particularly beneficial in immune disorders including autoimmune conditions.

Therefore, PKB inhibitors could be useful in the treatment of diseases in which there is a disorder of proliferation, apoptosis or differentiation.

PKB inhibitors may also be useful in diseases resulting from insulin resistance and insensitivity, and the disruption of glucose, energy and fat storage such as metabolic disease and obesity.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoetic malignancy for example acute myeloid leukaemia, acute promyelocytic leukaemia, acute lymphoblastic leukaemia, chronic myeloid leukaemia, chronic lymphocytic leukaemia and other B-cell lymphoproliferative diseases, myelodysplastic syndrome, T-cell lymphoproliferative diseases including those derived from Natural Killer cells, Non-Hodgkin's lymphoma and Hodgkin's disease; Bortezomib sensitive and refractory multiple myeloma; hematopoetic diseases of abnormal cell proliferation whether pre malignant or stable such as myeloproliferative diseases including polycythemia vera, essential thrombocythemia and primary myelofibrosis; hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

A further subset of cancers includes breast cancer, ovarian cancer, prostate cancer, endometrial cancer and glioma.

It is also possible that some protein kinase B inhibitors can be used in combination with other anticancer agents. For example, it may be beneficial to combine of an inhibitor that induces apoptosis with another agent which acts via a different mechanism to regulate cell growth thus treating two of the characteristic features of cancer development. Examples of such combinations are set out below.

### Immune Disorders

Immune disorders for which PKA and PKB inhibitors may be beneficial include but are not limited to autoimmune conditions and chronic inflammatory diseases, for example systemic lupus erythematosus, autoimmune mediated glomerulonephritis, rheumatoid arthritis, psoriasis, inflammatory bowel disease, and autoimmune diabetes mellitus, Eczema hypersensitivity reactions, asthma, COPD, rhinitis, and upper respiratory tract disease.

### Other Therapeutic Uses

PKB plays a role in apoptosis, proliferation, differentiation and therefore PKB inhibitors could also be useful in the treatment of the following diseases other than cancer and those associated with immune dysfunction; viral infections, for example herpes virus, pox virus, Epstein-Barr virus, Sindbis virus, adenovirus, HIV, HPV, HCV and HCMV; prevention of AIDS development in HIV-infected individuals; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, degenerative diseases of the musculoskeletal system, for example, osteoporosis and arthritis, aspirin-sensitive rhinosinusitis, cystic fibrosis, multiple sclerosis, kidney diseases.

### Advantages of Compounds of the Invention

Compounds of the formula (I) and sub-groups thereof as defined herein will have advantages over prior art compounds.

In particular, the compounds of formulae (II), (II), (III), (IV), (IVa), (IVb) and (V) have advantages over prior art compounds.

Potentially the compounds of the invention have physiochemical properties suitable for oral exposure.

Compounds of the formula (I) should exhibit improved oral bioavailability relative to prior art compounds. Oral bioavailability can be defined as the ratio (F) of the plasma exposure of a compound when dosed by the oral route to the plasma exposure of the compound when dosed by the intravenous (i.v.) route, expressed as a percentage.

Compounds having an oral bioavailability (F value) of greater than 30%, more preferably greater than 40%, are particularly advantageous in that they may be administered orally rather than, or as well as, by parenteral administration.

Furthermore, compounds of the invention are both more potent and more selective in their activities against different kinases, and demonstrate enhanced selectivity for and potency against PKB in particular.

Compounds of the invention are advantageous over prior art compounds in that they have different susceptibilities to P450 enzymes and in that they exhibit improvements with regard to drug metabolism and pharmacokinetic properties.

Furthermore compounds of the invention should exhibit reduced dosage requirements.

Compounds of the invention are advantageous in that they have improved thermodynamic solubilities, thereby leading potentially to an improved dose: solubility ratio and reduced development risk.

Compounds of the invention also demonstrate improved cell activity in proliferation and clonogenic assays thereby indicating improved anti-cancer activity.

Compounds of the invention are potentially less toxic than prior art compounds.

hERG

In the late 1990s a number of drugs, approved by the US FDA, had to be withdrawn from sale in the US when it was discovered they were implicated in deaths caused by heart malfunction. It was subsequently found that a side effect of these drugs was the development of arrhythmias caused by the blocking of hERG channels in heart cells. The hERG channel is one of a family of potassium ion channels the first member of which was identified in the late 1980s in a mutant Drosophila melanogaster fruitfly (see Jan, L. Y. and Jan, Y. N. (1990). A Superfamily of Ion Channels. Nature, 345(6277):672). The biophysical properties of the hERG potassium ion channel are described in Sanguinetti, M. C., Jiang, C., Curran, M. E., and Keating, M. T. (1995). A Mechanistic Link Between an Inherited and an Acquired Cardiac Arrhythmia: HERG encodes the Ikr potassium channel. Cell, 81:299-307, and Trudeau, M. C., Warmke, J. W., Ganetzky, B., and Robertson, G. A. (1995). HERG, a Human Inward Rectifier in the Voltage-Gated Potassium Channel Family. Science, 269:92-95.

The elimination of hERG blocking activity remains an important consideration in the development of any new drug.

Compounds of formula (I) have reduced, negligible or no hERG ion channel blocking activity.

### Methods of Treatment

It is envisaged that the compounds of the formula (I) and sub-groups thereof as defined herein will be useful in the prophylaxis or treatment of a range of disease states or conditions mediated by protein kinase A and/or protein kinase B. Examples of such disease states and conditions are set out above.

The compounds are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example.

The compounds of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently, i.e. taken continuously for a given period such as a week, then discontinued for a period such as a week and then taken continuously for another period such as a week and so on throughout the duration of the treatment regimen. Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles.

In one particular dosing schedule, a patient will be given an infusion of a compound of the formula (I) for periods of one hour daily for up to ten days in particular up to five days for one week, and the treatment repeated at a desired interval such as two to four weeks, in particular every three weeks.

More particularly, a patient may be given an infusion of a compound of the formula (I) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

The compounds as defined herein can be administered as the sole therapeutic agent or they can be administered in combination therapy with one of more other compounds for treatment of a particular disease state, for example a neoplastic disease such as a cancer as hereinbefore defined. Examples of other therapeutic agents or treatments that may be administered together (whether concurrently or at different time intervals) with the compounds of the formula (I) include but are not limited to:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Chromatin targeted therapies
Radiotherapy, and,
Other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF). Also included are agents that inhibit bone resorption such as bisphosphonate agents e.g. zoledronate, pamidronate and ibandronate, agents that suppress inflammatory responses (such as dexamethazone, prednisone, and prednisolone) and agents used to reduce blood levels of growth hormone and IGF-I in acromegaly patients such as synthetic forms of the brain hormone somatostatin, which includes octreotide acetate which is a long-acting octapeptide with pharmacologic properties mimicking those of the natural hormone somatostatin. Further included are agents such as leucovorin, which is used as an antidote to drugs that decrease levels of folic acid, or folinic acid it self and agents such as megestrol acetate which can be used for the treatment of side-effects including oedema and thromoembolic episodes.

Each of the compounds present in the combinations of the invention may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

### Methods of Diagnosis

Prior to administration of a compound of the formula (I), a patient may be screened to determine whether a disease or condition from which the patient is or may be suffering is one which would be susceptible to treatment with a compound having activity against protein kinase A and/or protein kinase B.

For example, a biological sample taken from a patient may be analysed to determine whether a condition or disease, such as cancer, that the patient is or may be suffering from is one which is characterised by a genetic abnormality or abnormal protein expression which leads to up-regulation of PKA and/or PKB or to sensitisation of a pathway to normal PKA and/or PKB activity, or to upregulation of a signal transduction component upstream of PKA and/or PKB such as, in the case of PKB, PI3K, GF receptor and PDK 1 & 2.

Alternatively, a biological sample taken from a patient may be analysed for loss of a negative regulator or suppressor of the PKB pathway such as PTEN. In the present context, the term “loss” embraces the deletion of a gene encoding the regulator or suppressor, the truncation of the gene (for example by mutation), the truncation of the transcribed product of the gene, or the inactivation of the transcribed product (e.g. by point mutation) or sequestration by another gene product.

The term up-regulation includes elevated expression or over-expression, including gene amplification (i.e. multiple gene copies) and increased expression by a transcriptional effect, and hyperactivity and activation, including activation by mutations. Thus, the patient may be subjected to a diagnostic test to detect a marker characteristic of up-regulation of PKA and/or PKB. The term diagnosis includes screening. By marker we include genetic markers including, for example, the measurement of DNA composition to identify mutations of PKA and/or PKB The term marker also includes markers which are characteristic of up regulation of PKA and/or PKB and/or other factors which lead to an upregulation of the relevant pathways, including enzyme activity, enzyme levels, enzyme state (e.g. phosphorylated or not) and mRNA levels of the aforementioned proteins.

The above diagnostic tests and screens are typically conducted on a biological sample selected from tumour biopsy samples, blood samples (isolation and enrichment of shed tumour cells), stool biopsies, sputum, chromosome analysis, pleural fluid, peritoneal fluid, bone marrow or urine.

Identification of an individual carrying a mutation in PKA and/or PKB or a rearrangement of TCL-1 or loss of PTEN expression may mean that the patient would be particularly suitable for treatment with a PKA and/or PKB inhibitor. Tumours may preferentially be screened for presence of a PKA and/or PKB variant prior to treatment. The screening process will typically involve direct sequencing, oligonucleotide microarray analysis, or a mutant specific antibody.

Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of PKB, or detection of PKB variants could be applicable in the present case.

Therefore all of these techniques could also be used to identify tumours particularly suitable for treatment with PKA and/or PKB inhibitors.

For example, as stated above, PKB beta has been found to be upregulated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330). Therefore it is envisaged that PKB inhibitors, and in particular inhibitors of PKB beta, may be used to treat ovarian and pancreatic cancers.

PKB alpha is amplified in human gastric, prostate and breast cancer (Staal 1987, PNAS 84, 5034-5037; Sun et al 2001, Am. J. Pathol. 159, 431-437). Therefore it is envisaged that PKB inhibitors, and in particular inhibitors of PKB alpha, may be used to treat human gastric, prostate and breast cancer.

Increased PKB gamma activity has been observed in steroid independent breast and prostate cell lines (Nakatani et al 1999, J. Biol. Chem. 274, 21528-21532). Therefore it is envisaged that PKB inhibitors, and in particular inhibitors of PKB gamma, may be used to treat steroid independent breast and prostate cancers.

### EXPERIMENTAL

The invention will now be illustrated, but not limited, by reference to the specific embodiments described in the following procedures and examples.

The starting materials for each of the procedures described below are commercially available unless otherwise specified.

Proton magnetic resonance (¹H NMR) spectra were recorded on a Bruker AV400 instrument operating at 400.13 MHz, in Me-d₃-OD at 27° C., unless otherwise stated and are reported as follows: chemical shift δ/ppm (number of protons, multiplicity where s=singlet, d=doublet, t=triplet, q=quartet, m=multiplet, br=broad). The residual protic solvent MeOH (δ_{H}=3.31 ppm) was used as the internal reference.

In the examples, the compounds prepared were characterised by liquid chromatography and mass spectroscopy using the systems and operating conditions set out below. Where chlorine is present, the mass quoted for the compound is for ³⁵Cl. The operating conditions used are described below.

### LCT System 1

HPLC System: Waters Alliance 2795 Separations Module

Mass Spec Detector: Waters/Micromass LCT

UV Detector: Waters 2487 Dual λ Absorbance Detector

### Polar Analytical Conditions:

| Eluent A: | Methanol | |
|---|---|---|
| Eluent B: | 0.1% Formic Acid in Water | |
| Gradient: | | |
| Time (mins) | A | B |
| 0 | 10 | 90 |
| 0.5 | 10 | 90 |
| 6.5 | 90 | 10 |
| 10 | 90 | 10 |
| 10.5 | 10 | 90 |
| 15 | 10 | 90 |

| | |
|---|---|
| Flow: | 1.0 ml/min |
| Column: | Supelco DISCOVERY C₁₈ 5 cm × 4.6 mm i.d., 5 μm |

### MS Conditions:

Capillary voltage: 3500 v (+ve ESI), 3000 v (−ve ESI)

Cone voltage: 40 v (+ve ESI), 50 v (−ve ESI)

Source Temperature: 100° C.

Scan Range: 50-1000 amu

Ionisation Mode: +ve/−ve electrospray ESI (Lockspray™)

### LCT System 2

HPLC System: Waters Alliance 2795 Separations Module

Mass Spec Detector: Waters/Micromass LCT

UV Detector: Waters 2487 Dual λ Absorbance Detector

### Analytical Conditions:

| Eluent A: | Methanol | |
|---|---|---|
| Eluent B: | 0.1% Formic Acid in Water | |
| Gradient: | | |
| Time (mins) | A | B |
| 0 | 10 | 90 |
| 0.6 | 10 | 90 |
| 1.0 | 20 | 80 |
| 7.5 | 90 | 10 |
| 9 | 90 | 10 |
| 9.5 | 10 | 90 |
| 10 | 10 | 90 |

| | |
|---|---|
| Flow: | 1 ml/min |
| Column: | Supelco DISCOVERY C₁₈ 5 cm × 4.6 mm i.d., 5 μm |

### MS Conditions:

Capillary voltage: 3500 v (+ve ESI), 3000 v (−ve ESI)

Cone voltage: 40 v (+ve ESI), 50 v (−ve ESI)

Source Temperature: 100° C.

Scan Range: 50-1000 amu

Ionisation Mode: +ve/−ve electrospray ESI (Lockspray™)

### LCT System 3

HPLC system: Waters alliance 2795 Separations Module

Mass Spec Detector: Waters/Micromass LCT

UV Detector: Waters 2478 Dual γ Absorbance Detector

### Analytical Conditions

| Eluent A: | Methanol | |
|---|---|---|
| Eluent B: | 0.1% Formic Acid in Water | |
| Gradient | | |
| Time (mins) | A | B |
| 0 | 10 | 90 |
| 0.3 | 10 | 90 |
| 0.6 | 20 | 80 |
| 4.5 | 90 | 10 |
| 5.4 | 90 | 10 |
| 5.7 | 10 | 90 |
| 6.0 | 10 | 90 |

| | |
|---|---|
| Flow: | 1 mL/min |
| Column: | Supelco DISCOVERY C₁₈ 3 cm × 4.6 mm i.d., 3 μm |

(MS conditions as before)

In the examples below, the following key is used to identify the LCMS conditions

| | |
|---|---|
| LCT1 | LCT System 1 - polar analytical conditions |
| LCT2 | LCT System 2 - polar analytical conditions |
| LCT3 | LCT System 3 - polar analytical conditions |

### Example 1

### 4-(4-amino-piperidin-1-yl)-1,3-dihydro-pyrrolo[2,3-b]pyridin-2-one

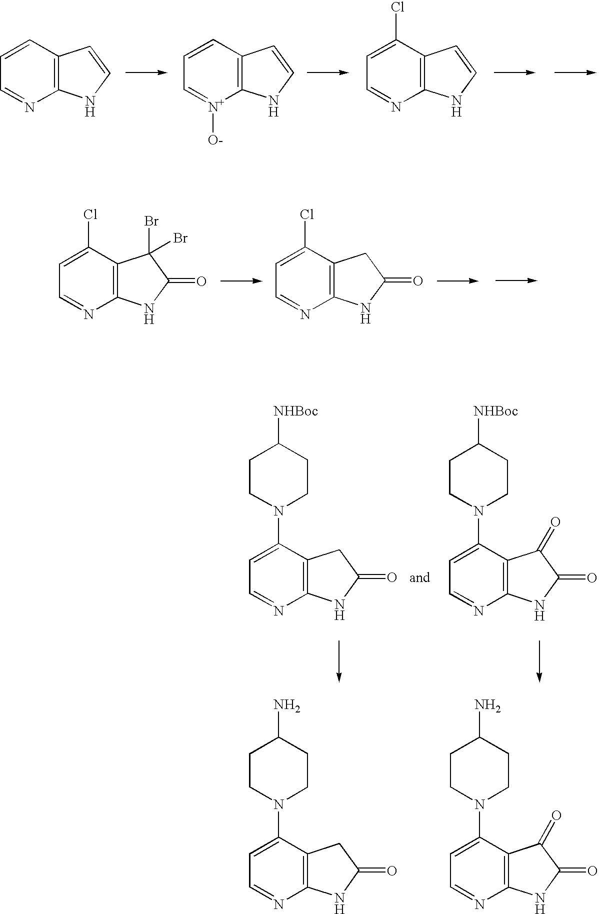

### 1A. 1H-Pyrrolo[2,3-b]pyridine 7-oxide

A solution of 1H-pyrrolo[2,3-b]pyridine (6.35 g, 53 mmol) in ethyl acetate (200 mL) was cooled to 0-5° C. in an ice bath. To the cooled solution was added mCPBA (14 g, 64 mmol) over 10 min. The resulting solution was warmed to room temperature until the starting material was totally consumed (2.5 h). The resulting slurry was filtered to collect the N-oxide as the meta-chlorobenzoic acid salt. The solid was washed with additional ethyl acetate and dried to provide 10.4 g (36 mmol). A suspension of the 7-hydroxy-1H-pyrrolo[2,3-b]pyridinium m-chlorobenzoate (10.4 g, 36 mmol) in water (100 mL) was basified to pH 11 with saturated aqueous K₂CO₃. The mixture was cooled (+4° C.) overnight to give crystals which were collected and washed with hexane followed by diethyl ether to yield 1H-pyrrolo[2,3-b]pyridine 7-oxide (3.22 g, 24 mmol, 67%). LC-MS (LCT1) m/z 135.1 [M+H⁺], Rₜ 2.62 min.

### 1B. 4-Chloro-1H-pyrrolo[2,3-b]pyridine

Methanesulphonyl chloride (5 mL, 64 mmol) was added dropwise to a solution of 1H-pyrrolo[2,3-b]pyridine 7-oxide (3.18 g, 24 mmol) in DMF (16 mL) heated to 50° C. The resulting mixture was heated at 72° C. overnight. The reaction mixture was cooled to 30° C. and quenched with water (50 mL). The mixture was cooled in an ice bath and sufficient 10M aqueous NaOH was added to raise the pH to 7. The resulting slurry was warmed to room temperature, stirred for 15 min, and then filtered to collect the product. The solid was washed with water and dried in vacuo to give 4-chloro-1H-pyrrolo[2,3-b]pyridine (2.97 g, 19.5 mmol, 81%). LC-MS (LCT1) m/z 153.03 [M+H⁺], Rₜ 5.77 min.

### 1C. 3,3-Dibromo-4-chloro-1,3-dihydro-pyrrolo[2,3-b]pyridin-2-one

To a stirred solution of 4-chloro-1H-pyrrolo[2,3-b]pyridine (1 g, 6.5 mmol) in 50 mL of t-butanol was added in small portions pyridinium tribromide 90% (7.24 g, 22.6 mmol) over 7 min. The reaction was stirred at room temperature overnight. t-Butanol was removed in vacuo and the resulting residue was dissolved in ethyl acetate-water (200 mL:200 mL). The organic layer was separated and the aqueous layer was further extracted with ethyl acetate (2×100 mL). The combined organic extracts were washed with water, brine, dried (Mg₂SO₄) and concentrated in vacuo to give 3,3-dibromo-4-chloro-1,3-dihydro-pyrrolo[2,3-b]pyridin-2-one (2.17 g, 6.6 mmol, 100%). LC-MS (LCT1) m/z 326.78 [M+H⁺], Rₜ 5.75 min.

### 1D. 4-Chloro-1,3-dihydro-pyrrolo[2,3-b]pyridin-2-one

A suspension of 3,3-dibromo-4-chloro-1,3-dihydro-pyrrolo[2,3-b]pyridin-2-one (1.05 g, 3.15 mmol), ethanol (120 mL) and 10% Pd/C (391 mg) was hydrogenated at room temperature and room pressure for 6 h 15 min. The reaction mixture was filtered through a pad of celite and washed with methanol. The solvents were evaporated and the crude material was partitioned between dichloromethane (50 mL) and saturated aqueous sodium bicarbonate (50 mL). After separating the two phases the aqueous layer was further extracted with dichloromethane (2×50 mL). The combined organic layers were dried (Mg₂SO₄), filtered and evaporated to give 4-chloro-1,3-dihydro-pyrrolo[2,3-b]pyridin-2-one (328 mg, 1.94 mmol, 62%). LC-MS (LCT: 15 min run) m/z 169.02 [M+H⁺], Rₜ 3.96 min.

### 1E. [1-(2-oxo-2,3-dihydro-1H-pyrrolo[2,3-b]pyridin-4-yl)-piperidin-4-yl]-carbamic acid tert-butyl ester

A mixture of 4-chloro-1,3-dihydro-pyrrolo[2,3-b]pyridin-2-one (25 mg, 0.15 mmol), piperidin-4-yl-carbamic acid tert-butyl ester (38 mg, 0.3 mmol) and N-methyl-pyrrolidinone (0.2 ml) was irradiated in the microwave for 1 h at 155° C. The solution was diluted in methanol and purified on SCX-II acidic resin eluting with methanol and then with 2M ammonia—methanol. The crude material was further purified by flash silica column chromatography, eluting with 10% methanol—dichloromethane, to afford a mixture of [1-(2,3-dioxo-2,3-dihydro-1H-pyrrolo[2,3-b]pyridin-4-yl)-piperidin-4-yl]-carbamic acid tert-butyl ester and [1-(2-oxo-2,3-dihydro-1H-pyrrolo[2,3-b]pyridin-4-yl)-piperidin-4-yl]-carbamic acid tert-butyl ester. These compounds were separated by preparative HPLC (Discovery C18 Supelco HPLC column 15 cm×10 mm, 5 μL; acetonitrile/water gradient solvent system).

[1-(2-oxo-2,3-dihydro-1H-pyrrolo[2,3-b]pyridin-4-yl)-piperidin-4-yl]-carbamic acid tert-butyl ester: 1.6 mg, 0.005 mmol, 3%. LC-MS (LCT2) m/z 333.27 [M+H+], Rₜ 3.43 min

### 1F. 4-(4-Amino-piperidin-1-yl)-1,3-dihydro-pyrrolo[2,3-b]pyridin-2-one

Trifluoroacetic acid (0.5 ml, 6.7 mmol) was added dropwise to a solution of 1-(2-oxo-2,3-dihydro-1H-pyrrolo[2,3-b]pyridin-4-yl)-piperidin-4-yl]-carbamic acid tert-butyl ester in dichloromethane (1 mL). The solution was stirred at rt for 45 min. The solvents were concentrated and the crude mixture was purified on SCX-II acidic resin, eluting with methanol then 2M ammonia—methanol, to give the title compound. LC-MS (LCT2) m/z 233.22 [M+H⁺], Rₜ 0.63 min. ¹H NMR (500 MHz, MeOD) δ 7.81 (d, J=5 Hz, 1H), 6.57 (d, J=5 Hz, 1H), 3.90-3.95 (m, 2H), 3.61-3.65 (m, 2H), 2.99-3.09 (m, 3H), 1.92-2.00 (m, 2H), 1.32-1.52 (m, 2H).

### Example 2

### 4-Amino-1-(7H-pyrrolo[2,3-d]pyrimidin-4-yl)-piperidine-4-carboxylic acid (6-chloro-biphenyl-3-ylmethyl)-amide hydrochloride

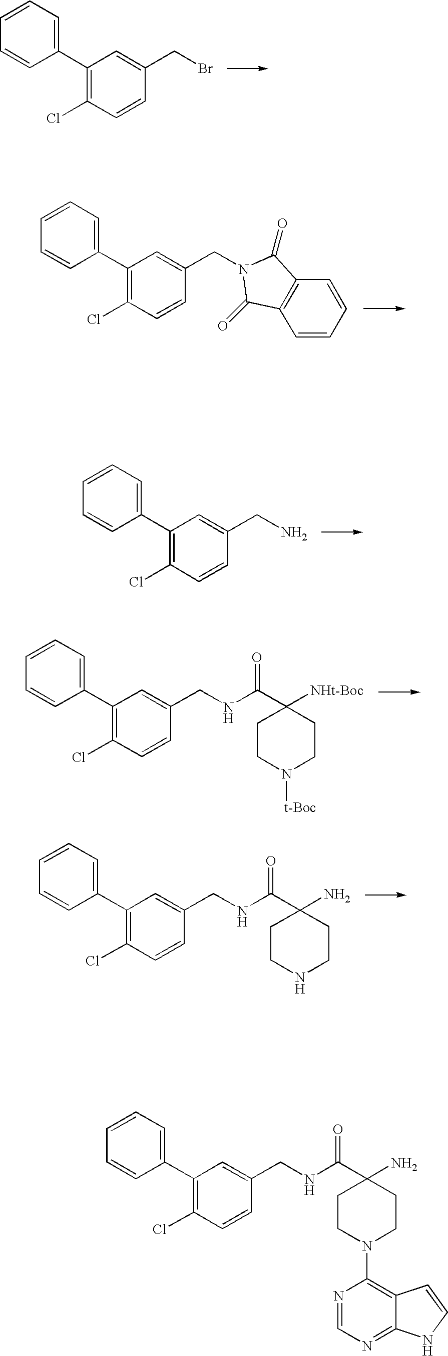

### 2A. 2-Chloro-5-methyl-biphenyl

A mixture of 2-bromo-1-chloro-4-methyl-benzene (4.83 g, 23.4 mmol), benzeneboronic acid (5.7 g, 46.7 mmol), Pd(Ph₃ P)₄ (1.35 g, 1.2 mmol) and 2M Na₂CO₃ (34 mL) in DME (100 mL) was stirred at 100° C. under N₂ for 16 h. After cooling, the resulting suspension was filtered. The filtrate was diluted with saturated brine and extracted with ethyl acetate (2×150 mL). The combined organic layers were washed with brine (100 mL) and water (100 mL), dried (Na₂SO₄) and filtered through decolourising charcoal. After evaporation of the solvent, n-hexane was added to the oil. The mixture was filtered to remove solids and the filtrate was concentrated. The resulting crude oil was purified by silica column chromatography (ethyl acetate:n-hexane/1:20) to give the title compound (1.76 g, 90%) as a light yellow oil. Rf=0.55. GC-MS (EI) m/z 202.3 [M]⁺, Rₜ 3.41 min. ¹H (500 MHz, CDCl₃) δ 7.64 (1H, d, J=7.5 Hz), 7.50-7.35 (5H, m), 7.15 (1H, s), 7.12 (1H, d, J=7.5 Hz), 2.39 (3H, s).

### 2B. 5-Bromomethyl-2-chloro-biphenyl

A mixture of 2-chloro-5-methyl-biphenyl (0.99 g, 4.9 mmol), NBS (1.04 g, 5.8 mmol) and AIBN (80 mg, 0.49 mmol) in CCl₄ was irradiated by a 500 W lamp, while heated at 80° C., for 5 h. After cooling, the suspension was filtered. The filtrate was concentrated and the resulting crude oil was purified by silica column chromatography (ethyl acetate:n-hexane/1:9) to give the title compound (1.02 g, 74%) as a light yellow oil. GC-MS (EI) m/z 281.9 [M]⁺, Rₜ 4.25 min. ¹H (500 MHz, CDCl₃) δ 7.55-7.30 (8H, m), 4.50 (2H, s).

### 2C. 2-(6-Chloro-biphenyl-3-ylmethyl)-isoindole-1,3-dione

A mixture of 5-bromomethyl-2-chloro-biphenyl (0.29 g, 1.0 mmol), potassium phthalimide (0.22 g, 1.2 mmol) and 18-crown-6 ether (85 mg, 0.32 mmol) in DMF (6 mL) was stirred at 100° C. for 16 h. After cooling, the mixture was diluted with brine and extracted with ethyl acetate (2×50 mL). The combined organic layers were washed with brine and water. The organic layers were dried, filtered and evaporated to give a yellow oil (0.33 g, 90%) that was used without further purification. ¹H (CDCl₃) δ 7.85-7.35 (12H, m), 4.86 (2H, s).

### 2D. C-(6-Chloro-biphenyl-3-yl)-methylamine

Hydrazine hydrate (0.2 mL) was added to a suspension of 2-(6-chloro-biphenyl-3-ylmethyl)-isoindole-1,3-dione (20 mg, 0.06 mmol) in methanol (4 mL). The reaction mixture was stirred at rt for 16 h. Solvent was evaporated and the solid was purified on SCX-II acidic resin, eluting with ammonia-methanol to obtain a yellow oil (10 mg, 80%). LC-MS (LCT2) m/z 201.1 [M-NH₂]⁺, Rₜ 3.84 min. ¹H (500 MHz, CDCl₃) δ 7.35-7.15 (8H, m), 3.80 (2H, s), 2.40 (2H, s, broad).

### 2E. 4-tert-Butoxycarbonylamino-4-[(6-chloro-biphenyl-3-ylmethyl)-carbamoyl]-piperidine-1-carboxylic acid tert-butyl ester

Diisopropylethylamine (1.8 mL, 10.5 mmol) was added to a solution of 4-tert-butoxycarbonylamino-piperidine-1,4-dicarboxylic acid mono-tert-butyl ester (58 mg) and HATU in DMF under N₂ to give a yellow solution. After stirring at room temperature for 0.5 hr, C-(6-chloro-biphenyl-3-yl)-methylamine was added. The resulting solution was stirred for approximately 20 hours and the mixture was then diluted with brine (50 mL) and extracted with ethyl acetate (2×50 mL). The combined organic layers were washed with aqueous NaHCO₃ (2×50 mL), citric acid (50 mL) and brine (50 mL), then dried (Na₂SO₄). Solvent was removed by evaporation to give the title compound (90 mg, 98%). LC-MS (LCT2) m/z 544.3 [M+H]⁺, Rₜ 8.60 min. ¹H (500 MHz, CDCl₃) δ 7.40-7.10 (8H, m), 4.75 (1H, s, broad), 4.35 (2H, s), 3.75 (2H, m, broad), 3.02 (2H, m, broad), 2.00 (2H, m, broad), 1.90 (2H, m, broad), 1.38 (9H, s), 1.20 (9H, s).

### 2F. 4-Amino-piperidine-4-carboxylic acid (6-chloro-biphenyl-3-ylmethyl)-amide hydrochloride

4-tert-Butoxycarbonylamino-4-[(6-chloro-biphenyl-3-ylmethyl)-carbamoyl]-piperidine-1-carboxylic acid tert-butyl ester (90 mg) was stirred in a mixture of methanol (6 mL) and 4M HCl/dioxane (6 mL) at room temperature for 16 hours. The solid that formed was collected, washed in ether and dried in vacuo to give the title compound (45 mg, 65%) as a cream solid. LC-MS (LCT2) m/z 344.2 [M+H]⁺, Rₜ 3.10 min. ¹H (500 MHz, d₄-MeOD) δ: 7.48-7.32 (8H, m), 4.50 (2H, s), 3.46 (2H, m, broad), 3.35 (2H, m, broad), 2.66 (2H, m, broad), 2.22 (2H, m, broad).

### 2G. 4-Amino-1-(7H-pyrrolo[2,3-d]pyrimidin-4-yl)-piperidine-4-carboxylic acid (6-chloro-biphenyl-3-ylmethyl)-amide hydrochloride

A mixture of 4-chloro-7H-pyrrolo[2,3-d]pyrimidine (6 mg, 0.04 mmol), 4-amino-piperidine-4-carboxylic acid (6-chloro-biphenyl-3-ylmethyl)-amide hydrochloride (15 mg, 0.04 mmol), triethylamine (0.025 mL) in n-butanol (1.0 mL) was irradiated in a CEM microwave machine for 1 h at 100° C. (200 W) with cooling on. On completion, n-butanol was evaporated. The crude material was purified by preparative TLC (dichloromethane:methanol:ammonia/10:2:0.1). Then HCl in dioxane (2 mL) was added to the residue. After evaporating the solvent, the yellow solid was washed with ether and dried in vacuo (8 mg, 27%). LC-MS (LCT2) m/z 461.2 [M+H]⁺, Rₜ 4.53 min. Free base: ¹H (500 MHz, d₄-MeOD) δ 8.04 (1H, s), 7.32-7.12 (8H, m), 7.02 (1H, d, J=3.6 Hz), 6.50 (1H, d, J=3.6 Hz), 4.35 (2H, m, broad), 4.30 (2H, s), 3.55 (2H, m, broad), 2.14 (2H, m, broad), 1.54 (2H, m, broad).

### Example 3

### 4-Naphthalen-2ylmethyl-1-(7H-pyrrolo[2,3-d]pyrimidin-4-yl)piperidin-4-ylamine

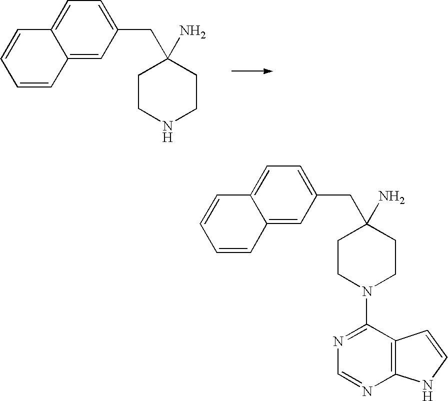

The title compound can be prepared from 4-chloro-7H-pyrrolo[2,3-d]pyrimidine and 4-(naphthalen-2-ylmethyl)piperidin-4-amine according to the method of Example 2G. LC-MS (LCT2) m/z 341 [M+H⁺], Rₜ 3.43 min. ¹H NMR (MeOD) δ 8.13 (1H, s), 7.84-7.72 (4H, m), 7.47-7.39 (3H, m), 7.11 (1H, d, J=3.5 Hz), 6.62 (1H, d, J=3.5 Hz), 4.32-4.29 (2H, m), 3.83-3.79 (2H, m), 2.97 (2H, s), 1.86-1.82 (2H, m), 1.62-1.59 (2H, m).

### Example 4

### C-[4-Biphenyl-3-yl-1-(7H-pyrrolo[2,3-d]pyrimidin-4-yl)piperidin-4-yl]methylamine

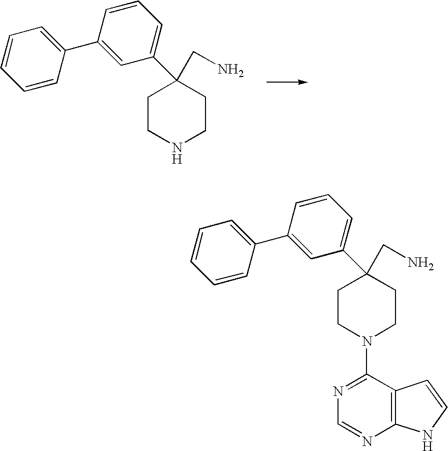

The title compound can be prepared from 4-chloro-7H-pyrrolo[2,3-d]pyrimidine and 4-(biphenyl-3-ylmethyl)piperidin-4-amine according to the method of Example 2G. LC-MS (LCT3) m/z 384 [M+H⁺], Rₜ 2.62 min. ¹H NMR (MeOD) δ 8.12 (1H, s), 7.68-7.33 (9H, m), 7.10 (1H, d, J=3.5 Hz), 6.62 (1H, J=3.5 Hz), 4.41-4.36 (2H, m), 3.74-3.72 (2H, m), 2.86 (2H, s), 2.47-2.45 (2H, m), 1.97-1.91 (2H, m).

### Example 5

### C-[4-(4′-Methoxybiphenyl-3-yl)-1-(7H-pyrrolo[2,3-d]pyrimidin-4-yl)piperidin-4-yl]methylamine

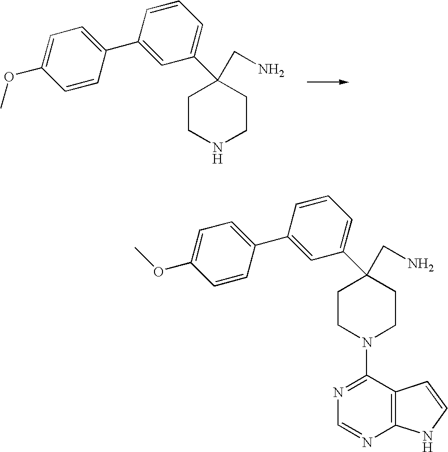

The title compound can be prepared from 4-chloro-7H-pyrrolo[2,3-d]pyrimidine and 4-((4′-methoxybiphenyl-3-yl)methyl)piperidin-4-amine using the method of Example 2G. LC-MS (LCT3) m/z 414 [M+H⁺], Rₜ 2.70 min. ¹H NMR (MeOD) δ 8.12 (1H, s), 7.66 (1H, s), 7.60-7.58 (2H, d, J=9.0 Hz), 7.52-7.50 (2H, m), 7.46-7.43 (1H, m), 7.12 (1H, d, J=3.5 Hz), 7.02 (2H, d, J=9.0 Hz), 6.66 (1H, d, J=3.5 Hz), 4.45-4.41 (2H, m), 3.85 (3H, s), 3.56-3.51 (2H, m), 2.88 (2H, s), 2.51-2.48 (2H, m), 1.98-1.92 (2H, m).

### Example 6

### 4-Amino-1-(8-oxo-8,9-dihydro-7H-purin-6-yl)-piperidine-4-carboxylic acid 4-chloro-benzylamide

### 6A. 5,6-Diamino-4-chloropyrimidine

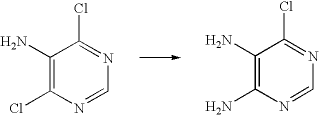

A mixture of 4,6-dichloro-5-aminopyrimidine (Aldrich Chemical Co.) (2.0 g, 12.2 mmol) and concentrated aqueous ammonia (20 ml) was heated to 100° C. in a sealed glass tube with vigorous stirring for 18 hours. The cooled tube was recharged with concentrated aqueous ammonia (8 ml), aggregates were broken up, and the mixture was reheated at 100° C. for a further 28 hours. The mixture was evaporated to dryness and the solids were washed with water (20 ml) and dried to give the product as yellow crystals (1.71 g, 97%). LC/MS (LCT1): Rₜ 1.59 [M+H]⁺ 147, 145.

### 6B. 6-Chloro-7,9-dihydropurin-8-one

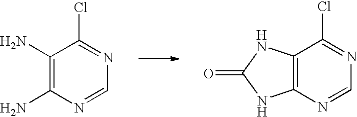

A mixture of the 5,6-diamino-4-chloropyrimidine of Example 6A (1.0 g, 6.92 mmol) and N,N′-carbonyldiimidazole (2.13 g, 13.2 mmol) in 1,4-dioxane (20 ml) was refluxed under argon for 48 hours. The solution was concentrated to a brown oil, which was triturated and washed with dichloromethane to give an off-white solid (1.02 g, 86%) LC/MS (LCT1): Rₜ 2.45 [M+H]⁺ 173, 171.

### 6C. 4-tert-Butoxycarbonylamino-4-(4-chloro-benzylcarbamoyl)-piperidine-1-carboxylic acid tert-butyl ester

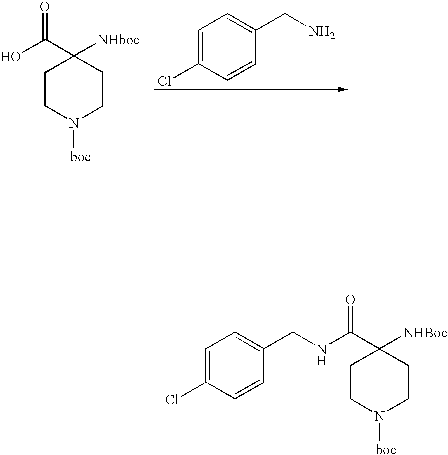

Dry DMF (1 mL) was added to a mixture of 4-tert-butoxycarbonylamino-piperidine-1,4-dicarboxylic acid mono tert-butyl ester (151 mg, 0.44 mmol) and HATU (220 mg, 0.58 mmol) under nitrogen. N-Ethyldiisopropylamine (0.38 mL, 2.1 mmol) was added to the solution and the reaction mixture was stirred for 15 min. 4-Chlorobenzylamine (70 uL, 0.57 mmol) was added and the solution was stirred for 23 h at rt and under nitrogen. The reaction mixture was partioned between dichloromethane (10 mL) and water (10 mL). The aqueous phase was further extracted with dichloromethane (20 mL). The combined organic layers were dried (Mg₂SO₄), filtered and concentrated. Flash column chromatography on silica, eluting with 4% methanol in dichloromethane, gave 4-tert-butoxycarbonylamino-4-(4-chloro-benzylcarbamoyl)-piperidine-1-carboxylic acid tert-butyl ester (177 mg, 0.38 mmol, 86%). LC-MS (LCT2) m/z 490 [M+Na⁺], Rₜ 8.09 min.

### 6D. 4-Amino-piperidine-4-carboxylic acid 4-chloro-benzylamide dihydrochloride

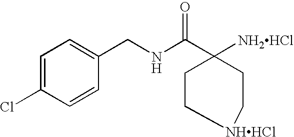

A 4M solution of HCl in dioxane (7.7 ml, 31 mmol) was added dropwise to a solution of 4-tert-butoxycarbonylamino-4-(4-chloro-benzylcarbamoyl)-piperidine-1-carboxylic acid tert-butyl ester (96 mg, 0.20 mmol) in methanol (7.7 mL) and stirred at rt for 17 h. The solvents were concentrated to give 4-amino-piperidine-4-carboxylic acid 4-chloro-benzylamide dihydrochloride (71 mg, 0.20 mmol, 100%) that was used in the next step without further purification.

¹H NMR (500 MHz, CD₃OD): 2.18 (2H, m), 2.64 (2H, m), 3.44 (4H, m), 4.47 (2H, s), 7.36 (4H, m).

### 6E. 4-Amino-1-(8-oxo-8,9-dihydro-7H-purin-6-yl)-piperidine-4-carboxylic acid 4-chloro-benzylamide

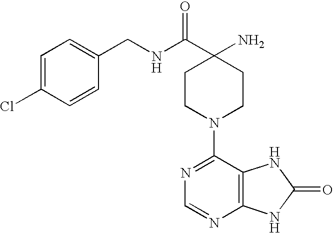

A degassed mixture of 4-amino-piperidine-4-carboxylic acid 4-chloro-benzylamide dihydrochloride (Example 6D) (116 mg, 0.32 mmol), 6-chloro-7,9-dihydro-purin-8-one (Example 6B) (50.5 mg, 0.30 mmol), triethylamine (0.3 mL, 2.14 mmol) and n-butanol (3 mL) was stirred at 100° C. for 18 h. The solvents were removed by evaporation and the crude material was purified on SCX-II acidic resin, eluting with methanol then 2M ammonia-methanol. The solid was recrystallized from methanol to give 4-amino-1-(8-oxo-8,9-dihydro-7H-purin-6-yl)-piperidine-4-carboxylic acid 4-chloro-benzylamide (45.5 mg, 0.11 mmol, 38%). LC-MS (LCT2) m/z 402.15 [M+H⁺], Rₜ 3.48 min. ¹H (500 MHz, DMSO) δ 11.37 (bs, 1H), 10.74 (bs, 1H), 8.53-8.55 (m, 1H), 8.06 (s, 1H), 7.36 (d, J=10 Hz, 2H), 7.26 (d, J=10 Hz, 2H), 4.26-4.27 (m, 2H), 3.95-4.06 (m, 2H), 3.34-3.39 (m, 2H), 3.17-3.18 (m, 2H), 1.93-1.98 (m, 2H), 1.38-1.40 (m, 2H).

### Example 7

### 4-Amino-1-(8-oxo-8,9-dihydro-7H-purin-6-yl)-piperidine-4-carboxylic acid 4-chloro-2-fluoro-benzylamide

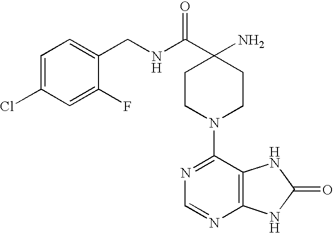

The title compound can be prepared according to the method of Example 6 using 4-amino-piperidine-4-carboxylic acid 4-chloro-2-fluoro-benzylamide and 6-chloro-7,9-dihydro-purin-8-one. LC-MS (LCT2) m/z 420.08 [M+H⁺], Rₜ 3.56 min. ¹H (500 MHz, DMSO) δ 11.43 (bs, 1H), 10.71 (bs, 1H), 8.57 (s, 1H), 8.06 (s, 1H), 7.24-7.45 (m, 3H), 4.25-4.28 (m, 2H), 3.95-4.11 (m, 2H), 3.17-3.34 (m, 4H), 1.90-1.95 (m, 2H), 1.33-1.39 (m, 2H).

### Example 8

### 6-[4-Amino-4-(2,4-dichlorobenzyl)piperidin-1-yl]-7,9-dihydropurin-8-one

### 8A. 4-(2,4-Dichlorobenzyl)piperidine-1,4-dicarboxylic acid 1-tert-butyl ester 4-methyl ester

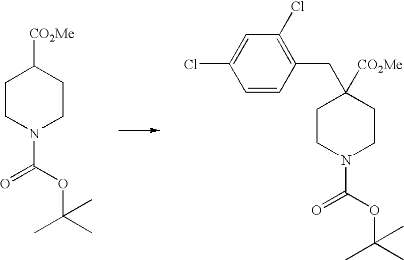

To a solution of isopropylamine (3.71 ml, 26.45 mmol) in THF (110 ml) at 0° C. is added n-butyllithium (10.1 ml of a 2.5M solution in hexanes, 25.25 mmol). The resulting LDA solution is added via cannula to a solution of piperidine-1,4-dicarboxylic acid 1-tert-butyl ester 4-methyl ester (5.85 g, 24.04 mmol) in THF (110 ml) and HMPA (20 ml) at −78° C. and stirring is continued for 1 hour. 2,4-Dichlorobenzyl chloride (50.49 mmol) in THF (20 ml) is added and the solution is warmed to room temperature over 2 hours. After stirring for 18 hours, saturated aqueous ammonium chloride (500 ml) is added and the aqueous phase is extracted with diethyl ether (2×200 ml). The organic phases are combined, dried over magnesium sulphate and concentrated to dryness. Purification by silica column chromatography (0.5% methanol in DCM) gives the title compound.

### 8B. 4-(2,4-Dichlorobenzyl)piperidine-1,4-dicarboxylic acid mono-tert-butyl ester

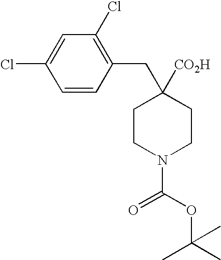

To a solution of 4-(2,4-dichlorobenzyl)piperidine-1,4-dicarboxylic acid 1-tert-butyl ester 4-methyl ester (4.117 mmol) in dioxane (20 ml), methanol (10 ml) and water (10 ml) at room temperature is added lithium hydroxide monohydrate (3.455 g, 82.341 mmol). After stirring at 50° C. for 2 days the solution is acidified to pH 6 with 2M HCl and the resulting precipitate is extracted with diethyl ether (2×100 ml). The organic phases are combined, dried over sodium sulphate and concentrated to dryness, to give the title compound.

### 8C. 4-(2,4-Dichlorobenzyl)piperidin-4-ylamine

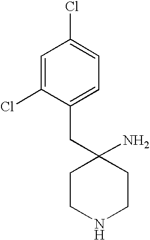

To a mixture of the product of Example 8B (4.126 mmol) and triethylamine (1.15 ml, 8.252 mmol) in THF (41 ml) at −15° C. is added isobutyl chloroformate (0.812 ml, 6.189 mmol). After 1 hour, a solution of sodium azide (0.536 g, 8.252 mmol) in water (10 ml) is added and the solution is warmed to room temperature overnight. Water (100 ml) is added and the aqueous phase is extracted with diethyl ether (3×50 ml). The organic phases are combined, washed with saturated sodium bicarbonate (50 ml) and dried over sodium sulphate. Toluene (100 ml) is added and the overall volume is reduced to approximately 90 ml. The resulting solution is warmed to 90° C. for 2 h, then cooled and added to 10% hydrochloric acid (70 ml). The biphasic mixture is warmed to 90° C. for 24 hours. The organic phase is separated and concentrated to dryness to give the crude amine salt.

The crude amine salt is dissolved in 2M NaOH (20 ml) and di-tert-butyl dicarbonate (1.61 g, 7.391 mmol) added. After 2 days the aqueous phase is extracted with diethyl ether (2×50 ml). The organic phases are combined, washed with 1M HCl (20 ml), saturated sodium bicarbonate (20 ml) and brine (20 ml), then dried over magnesium sulphate and concentrated. Purification by column chromatography (50% diethyl ether in hexanes) gives the doubly BOC-protected amine, which is subsequently deprotected by stirring with 4M HCl in dioxane (10 ml) and methanol (10 ml) at room temperature for 2 days. Concentration gives the title compound as the bis-hydrochloride salt.

### 8D. 6-[4-Amino-4-(2,4-dichlorobenzyl)piperidin-1-yl]-7,9-dihydropurin-8-one

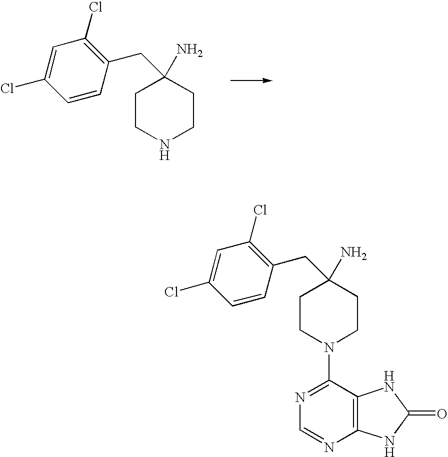

4-(2,4-Dichlorobenzyl)piperidin-4-ylamine (Example 8C) and 4-amino-4-(2,4-dichlorobenzyl)piperidine and 6-chloro-7,9-dihydro-purin-8-one (Example 6B) are reacted together according to the method of Example 6E to give the title compound. LC-MS (LCT2) m/z 393 [M+H⁺], Rₜ 4.15 min. ¹H NMR (DMSO) δ 8.03 (1H, s), 7.56 (1H, d, J=2.5 Hz), 7.45 (1H, d, J=8.5 Hz), 7.36 (1H, dd, J=8.5, 2.5 Hz), 3.93-3.91 (2H, m), 3.33-3.28 (2H, m), 2.79 (2H, s), 1.60-1.54 (2H, m), 1.35-1.32 (2H, m).

### Example 9

### 6-[4-Amino-4-(4-tert-butylbenzyl)piperidin-1-yl]-7,9-dihydropurin-8-one

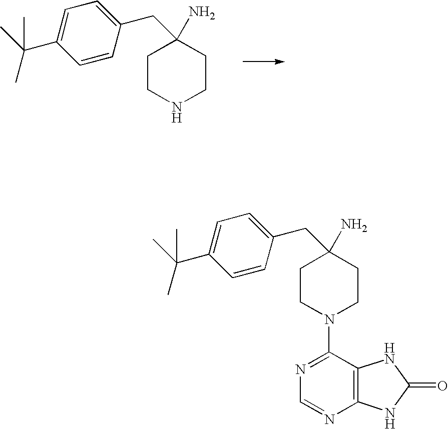

The title compound can be prepared according to the method of Example 8 but using 4-tert-butylbenzyl chloride in place of 2,4-dichlorobenzyl chloride in the first step. LC-MS (LCT2) m/z 381 [M+H⁺], Rₜ 4.72 min. ¹H (500 MHz, DMSO) δ 8.04 (1H, s), 7.29 (2H, d, J=8.0 Hz), 7.12 (2H, d, J=8.0 Hz), 3.90-3.87 (2H, m), 3.36-3.34 (2H, m), 2.58 (2H, s), 1.56-1.46 (2H, m), 1.29-1.26 (2H, m), 1.26 (9H, s).

### Example 10

### 6-[4-Aminomethyl-4-(4-trifluoromethoxyphenyl)piperidin-1-yl]-7,9-dihydropurin-8-one

### 10A. 4-(4-Trifluoromethoxyphenyl)-4-cyanopiperidin-1-carboxylic acid tert-butyl ester

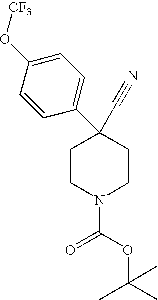

4-Trifluoromethoxyphenylacetonitrile is reacted with three equivalents of sodium hydride and one equivalent of N-tert-butyloxycarbonyl-bis-(2-chloroethyl)amine in DMF, initially at room temperature and then at 60° C. to give, after work up, the N-protected piperidine nitrile title compound.

### 10B. 4-Aminomethyl-4-(4-trifluoromethoxyphenyl)piperidine-1-carboxylic acid tert-butyl ester

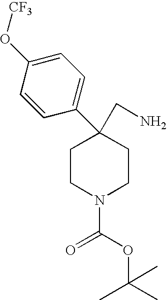

To a solution of 4-(4-trifluoromethoxyphenyl)-4-cyanopiperidin-1-carboxylic acid tert-butyl ester in ethanol (20 ml) at room temperature is added Raney Nickel (Raney Nickel 2800, 1 ml) and the suspension stirred under 1 atmosphere of hydrogen for 20 hours. The suspension is filtered through Celite and the filtrate concentrated to give the title amine.

### 10C. C-[4-(4-trifluoromethoxyphenyl)piperidin-4-yl]methylamine hydrochloride

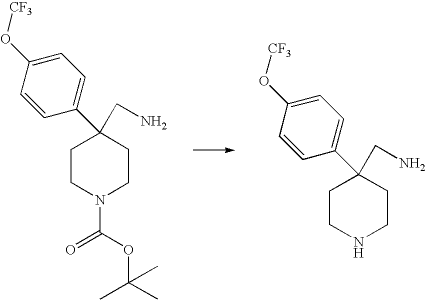

To a solution of 4-aminomethyl-4-(4-trifluoromethoxyphenyl)piperidine-1-carboxylic acid tert-butyl ester in methanol (10 ml) at room temperature is added 2M hydrochloric acid (10 ml). After 18 h the solution is concentrated to dryness to give the title amine.

### 10D. 6-[4-Aminomethyl-4-(4-trifluoromethoxyphenyl)piperidin-1-yl]-7,9-dihydropurin-8-one

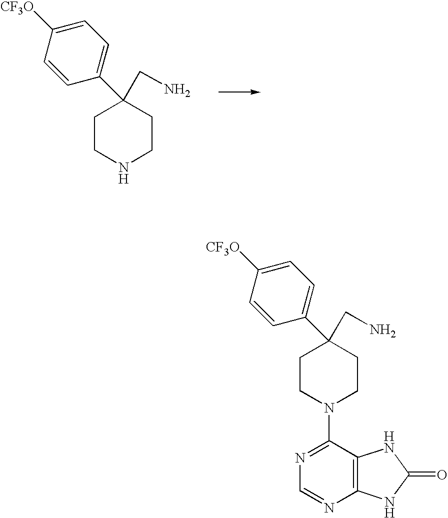

The title compound was prepared from C-[4-(4-trifluoromethoxyphenyl)piperidin-4-yl]methylamine hydrochloride (Example 10C) and 6-chloro-7,9-dihydropurin-8-one (Example 6B) according to the method of Example 6E. LC-MS (LCT3) m/z 409 [M+H⁺], Rₜ 2.81 min. ¹H (500 MHz, MeOD) δ 8.10 (1H, s), 7.56 (2H, d, J=9.0 Hz), 7.33 (2H, d, J=9.0 Hz), 4.01-3.97 (2H, m), 3.36-3.28 (2H, m), 2.82 (2H, s), 2.36-2.33 (2H, m), 1.96-1.90 (2H, m).

### Example 11

### 4-(4-Chlorobenzyl)-1-(7H-pyrrolo[2,3-d]pyrimidin-4-yl)piperidin-4-ylamine

### 11A. 4-(4-Chlorobenzyl)piperidine-1,4-dicarboxylic acid 1-tert-butyl ester 4-methyl ester

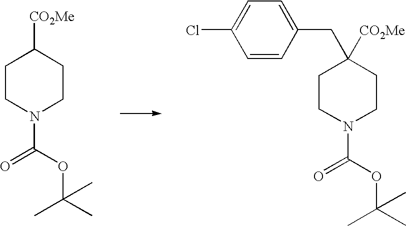

To a solution of isopropylamine (3.71 ml, 26.45 mmol) in THF (110 ml) at 0° C. was added n-butyllithium (10.1 ml of a 2.5M solution in hexanes, 25.25 mmol). The resulting LDA solution was added via cannula to a solution of piperidine-1,4-dicarboxylic acid 1-tert-butyl ester 4-methyl ester (5.85 g, 24.04 mmol) in THF (110 ml) and HMPA (20 ml) at −78° C. and stirring was continued for 1 hour. 4-Chlorobenzyl chloride (6.4 ml, 50.49 mmol) in THF (20 ml) was added and the solution was warmed to room temperature over 2 hours. After stirring for 18 hours, saturated aqueous ammonium chloride (500 ml) was added and the aqueous phase was extracted with diethyl ether (2×200 ml). The organic phases were combined, dried over magnesium sulphate and concentrated to dryness. Purification by silica column chromatography (0.5% methanol in DCM) gave the ester as an oil (3.03 g, 34%). LC-MS (LCT1) m/z 390 [M+Na⁺], Rₜ 8.02 min.

### 11B. 4-(4-Chlorobenzyl)piperidine-1,4-dicarboxylic acid mono-tert-butyl ester

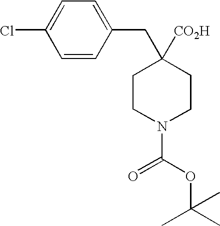

To a solution of 4-(4-chlorobenzyl)piperidine-1,4-dicarboxylic acid 1-tert-butyl ester 4-methyl ester (1.515 g, 4.117 mmol) in dioxane (20 ml), methanol (10 ml) and water (10 ml) at room temperature was added lithium hydroxide monohydrate (3.455 g, 82.341 mmol). After stirring at 50° C. for 2 days the solution was acidified to pH 6 with 2M HCl and the resulting white precipitate was extracted with diethyl ether (2×100 ml). The organic phases were combined, dried over sodium sulphate and concentrated to dryness, to give the acid as a white solid (1.460 g, 100%). LC-MS (LCT) m/z 376 [M+Na⁺], Rₜ 7.62 min.

### 11C. 4-(4-Chlorobenzyl)piperidin-4-yl amine

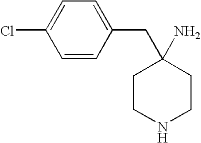

To a mixture of the acid (1.46 g, 4.126 mmol) and triethylamine (1.15 ml, 8.252 mmol) in THF (41 ml) at −15° C. was added isobutyl chloroformate (0.812 ml, 6.189 mmol). After 1 hour, a solution of sodium azide (0.536 g, 8.252 mmol) in water (10 ml) was added and the solution was warmed to room temperature overnight. Water (100 ml) was added and the aqueous phase was extracted with diethyl ether (3×50 ml). The organic phases were combined, washed with saturated sodium bicarbonate (50 ml) and dried over sodium sulphate. Toluene (100 ml) was added and the overall volume was reduced to approximately 90 ml. The resulting solution was warmed to 90° C. for 2 h, then cooled and added to 10% hydrochloric acid (70 ml). The biphasic mixture was warmed to 90° C. for 24 hours. The organic phase was separated and concentrated to dryness to give the crude amine salt (1.109 g).

The crude amine salt was dissolved in 2M NaOH (20 ml) and di-tert-butyl dicarbonate (1.61 g, 7.391 mmol) added. After 2 days the aqueous phase was extracted with diethyl ether (2×50 ml). The organic phases were combined, washed with 1M HCl (20 ml), saturated sodium bicarbonate (20 ml) and brine (20 ml), then dried over magnesium sulphate and concentrated. Purification by column chromatography (50% diethyl ether in hexanes) gave the doubly BOC-protected amine (0.685 g), which was subsequently deprotected by stirring with 4M HCl in dioxane (10 ml) and methanol (10 ml) at room temperature for 2 days. Concentration gave the desired amine as the bis-hydrochloride salt (0.492 g, 40% from acid).¹H NMR (MeOD) δ 7.48-7.44 (m, 2H), 7.35-7.32 (m, 2H), 3.53-3.47 (4H, m), 3.21 (s, 2H), 2.18-2.13 (4H, m).

### 11D. 6-[4-Amino-4-(4-chlorobenzyl)piperidin-1-yl]-7,9-dihydropurin-8-one

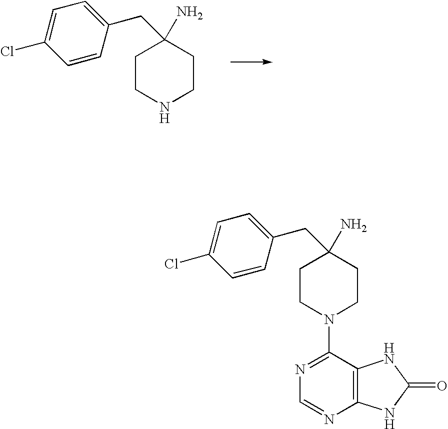

4-(4-Chlorobenzyl)piperidin-4-yl amine (Example 11C) and 6-chloro-7,9-dihydropurin-8-one (Example 6B) were reacted together according to the method in Example 6E to give the title compound. LC-MS (LCT2) m/z 359 [M+H⁺], Rₜ 3.61 min. ¹H (500 MHz, DMSO) δ 11.5 (1H, br s), 10.8 (1H, br s), 8.11 (1H, s), 7.44 (2H, d, J=8.5 Hz), 7.30 (2H, d, J=8.5 Hz), 3.77-3.65 (4H, m), 3.05 (2H, s), 1.79-1.68 (4H, m).

### Example 12

### 6-[4-Amino-4-(4-chlorophenyl)piperidin-1-yl]-7,9-dihydropurin-8-one

### 12A. 4-(4-Chlorophenyl)piperidine-1,4-dicarboxylic acid mono-tert-butyl ester

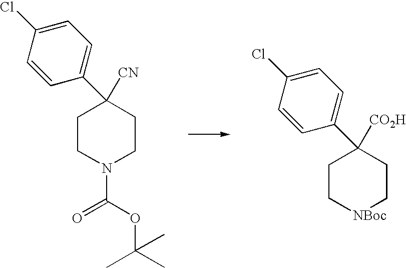

A solution of 4-(4-chlorophenyl)-4-cyanopiperidine-1-carboxylic acid tert-butyl ester (0.683 g, 2.129 mmol) in 6M HCl (20 ml) was refluxed for 4 days. The solution was cooled, basified with NaOH and di-tert-butyl dicarbonate (0.558 g, 2.555 mmol) added. After stirring for 24 h the solution was extracted with diethyl ether (2×75 ml). The organic phases were combined, washed with brine (50 ml), dried over magnesium sulphate and concentrated. Purification by silica column chromatography (5% methanol in DCM) gave the acid as a white foam (0.339 g, 47%). LC-MS (LCT2) m/z 362 [M+Na⁺], Rₜ 8.17 min.

### 12B. 4-(4-Chlorophenyl)piperidin-4-yl amine

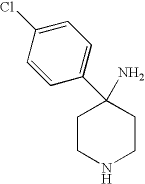

To a mixture of the product of Example 12A (4.126 mmol) and triethylamine (1.15 ml, 8.252 mmol) in THF (41 ml) at −15° C. is added isobutyl chloroformate (0.812 ml, 6.189 mmol). After 1 hour, a solution of sodium azide (0.536 g, 8.252 mmol) in water (10 ml) is added and the solution is warmed to room temperature overnight. Water (100 ml) is added and the aqueous phase is extracted with diethyl ether (3×50 ml). The organic phases are combined, washed with saturated sodium bicarbonate (50 ml) and dried over sodium sulphate. Toluene (100 ml) is added and the overall volume is reduced to approximately 90 ml. The resulting solution is warmed to 90° C. for 2 h, then cooled and added to 10% hydrochloric acid (70 ml). The biphasic mixture is warmed to 90° C. for 24 hours. The organic phase is separated and concentrated to dryness to give the crude amine salt.

The crude amine salt is dissolved in 2M NaOH (20 ml) and di-tert-butyl dicarbonate (1.61 g, 7.391 mmol) is added. After 2 days the aqueous phase is extracted with diethyl ether (2×50 ml). The organic phases are combined, washed with 1M HCl (20 ml), saturated sodium bicarbonate (20 ml) and brine (20 ml), then dried over magnesium sulphate and concentrated. Purification by column chromatography (50% diethyl ether in hexanes) gives the doubly BOC-protected amine which is subsequently deprotected by stirring with 4M HCl in dioxane (10 ml) and methanol (10 ml) at room temperature for 2 days. Concentration gives the desired amine as the bis-hydrochloride salt. ¹H NMR (MeOD) δ 7.74-7.70 (m, 2H), 7.65-7.61 (m, 2H), 3.61-3.52 (m, 2H), 3.07-2.93 (m, 4H), 2.56-2.44 (m, 2H).

### 12C. 6-[4-Amino-4-(4-chlorophenyl)piperidin-1-yl]-7,9-dihydropurin-8-one

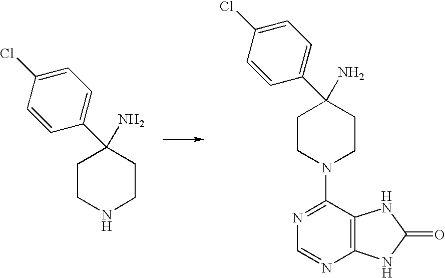

4-(4-Chlorophenyl)piperidin-4-yl amine (Example 12B) and 6-chloro-7,9-dihydropurin-8-one (Example 6B) were reacted together according to the method in Example 6E to give the title compound. LC-MS (LCT2) m/z 345 [M+H⁺], Rₜ 3.60 min. ¹H (500 MHz, DMSO) δ 8.07 (1H, s), 7.53 (2H, d, J=8.5 Hz), 7.34 (2H, d, J=8.5 Hz), 4.03-4.00 (2H, m), 3.51-3.46 (2H, m), 1.94-1.89 (2H, m), 1.63-1.60 (2H, m).

### Example 13

### 4-(4-tert-Butyl-benzyl)-1-(1H-pyrazolo[3,4-d]pyrimidin-4-yl)-piperidin-4-ylamine

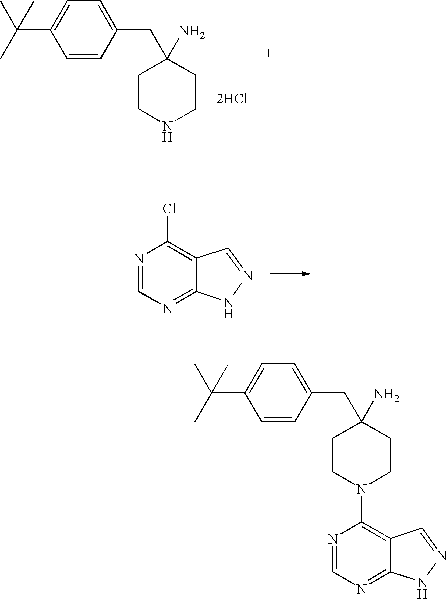

A mixture of 4-chloro-1H-pyrazolo[3,4-d]pyrimidine (40 mg, 0.26 mmol), 4-(4-tert-butyl-benzyl)-piperidin-4-ylamine dihydrochloride (Example 9) (82 mg, 0.26 mmol), triethylamine (0.2 mL) in ethanol (2.0 mL) was stirred at 80° C. for 16 h. Ethanol was removed by evaporation. The crude material was purified by silica column chromatography (dichloromethane:methanol: ammonia/10:2:0.1) to give a cream-coloured solid (11 mg, 12%). LC-MS (LCT2) m/z 365.3 [M+H⁺], Rₜ 4.48 min. ¹H (500 MHz, d₄-MeOD) δ 8.27 (1H, s), 8.26 (1H, s), 7.42 (2H, d, J=8.2 Hz), 7.24 (2H, d, J=8.2 Hz), 4.38 (2H, m), 3.92 (2H, m), 2.83 (2H, s), 1.84 (2H, m), 1.68 (2H, m), 1.38 (9H, s).

### General Methods

### Method A

### Boc Protection

To a solution of protected amine in a suitable organic solvent (e.g. dichloromethane, DMF, THF) was added a base (e.g. triethylamine, aqueous sodium hydroxide or aqueous sodium bicarbonate, 1 to excess equivalents) and di-tert-butyl dicarbonate (1 to excess equivalents). This mixture was stirred at room temperature for 30 minutes to 18 hours before aqueous workup. The crude product was optionally purified by silica column chromatography eluting with ethyl acetate/petroleum ether to furnish the desired compound.

### Method B

### Boronate Ester Formation

A mixture of a protected aryl halide (preferably an iodide or bromide, 1 equivalent), bis(pinacolato)diboron (1 equivalent), potassium acetate (3 equivalents) and [1,1′-bis(diphenylphosphino)ferrocene]dichloro palladium(II) (0.05 equivalents) in dimethylsulfoxide was heated to 80 deg C. under nitrogen for 2-18 hours. The reaction was then allowed to cool, diluted with ethyl acetate then filtered under suction. The resultant crude material was purified by tituration or silica column chromatography (typically with mixture of ethyl acetate/petrol) to furnish the desired compounds as solids.

### Method C1

Suzuki Coupling—With Microwave irradiation

A mixture of aryl chloride, bromide or iodide (1 equivalent), inorganic base (typically potassium carbonate or potassium phosphate, 2-6 equivalents), catalyst (bis(tri-t-butylphosphine)palladium (0) for coupling of aryl chlorides; tetrakis(triphenylphosphine)palladium (0) for coupling of aryl bromides or iodides) and 4-(4,4,5,5-tetramethyl-1,3,2-dioxaborolan-2-yl)-1H-pyrazole (1.1-1.5 equivalents) in ethanol/methanol/toluene/water (ca. equal proportions) was irradiated in a CEM Explorer™ microwave to 80-145° C. for 15-90 minutes using ≦100 watts power. The reaction was either concentrated in vacuo or directly partitioned between ethyl acetate and 2N NaOH or water. The aqueous layer was extracted with ethyl acetate and the combined organic layers were on occasions washed with brine, dried (MgSO₄) and concentrated under reduced pressure. In some cases the product precipitated out during work up, this was collected by filtration. If as this stage there was a significant amount of residual starting material, fresh reactants and reagents would be added and the reaction irradiated then worked up for a second time. The crude product was purified by column chromatography (SiO₂), eluting with a mixture of dichloromethane/methanol or dichloromethane/methanol/ammonia or dichloromethane/methanol/acetic acid/H₂O and/or via preparative HPLC to afford the desired compounds.

### Method C2

### Suzuki Coupling—Under Thermal Heating

Under this method, the Suzuki coupling exemplified in Method C1 was conducted as described in C1, but instead the reaction mixture was heated thermally from 50° C. to reflux for a period of 30 minutes to 16 hours.

### Method C3

### Suzuki Coupling—Microwave Irradiation II

A mixture of 6-chloro-7,9-dihydro-purin-8-one (Preparation A, 1-1.3 equivalent), inorganic base (typically potassium carbonate or potassium phosphate, 2-6 equivalents), catalyst (bis(tri-t-butylphosphine)palladium (0) and protected aryl halide (1 equivalents) in ethanol/methanol/toluene/water (ca. equal proportions) was irradiated in a CEM Explorer™ microwave to 80-145° C. for 15-30 minutes using ≦100 watts power. The reaction was either concentrated in vacuo or directly partitioned between ethyl acetate and 2N NaOH or water. The aqueous layer was extracted with ethyl acetate and the combined organic layers were on occasions washed with brine, dried (MgSO₄) and concentrated under reduced pressure. In some cases the product precipitated out during work up, this was collected by filtration. If as this stage there was a significant amount of residual starting material, fresh reactants and reagents would be added and the reaction irradiated then worked up for a second time. The crude product was purified by column chromatography (SiO₂), eluting with a mixture of dichloromethane/methanol or dichloromethane/methanol/ammonia or dichloromethane/methanol/acetic acid/H₂O or petrol/ethyl acetate and/or via preparative HPLC to afford the desired compounds.

### Method D

### Boc Deprotection

To the protected amine, optionally dissolved in a suitable organic solvent (typically dichloromethane), was added a strong organic (e.g. trifluoroacetic acid) or inorganic (e.g. hydrochloric acid in 1,4-dioxane) acid. This mixture was stirred at room temperature for between 10 minutes and 18 hours to furnish the crude amine as a salt. If necessary, purification could be achieved via silica column chromatography using a mixture of dichloromethane, methanol, acetic acid and H₂O or dichloromethane, methanol and ammonia, and/or via ion exchange chromatography and/or by preparative HPLC.

### Method E

### Acetonitrile Addition

To n-BuLi (2.5M in hexanes) (1.25 equivalents), in THF at −78° C., was added MeCN (1.25 equivalents). The mixture was stirred for 30 min at −78° C. followed by addition of a solution of the requisite benzophenone (1.0 equivalent) in THF. The mixture was then allowed to warm to r.t. over 30 min. after which saturated aqueous NH₄Cl was added. The organic layer was separated, washed with brine, dried (Na₂SO₄) and then concentrated in vacuo to furnish the desired compound, which was used in the next step without further purification.

### Method F1

### Nitrile Reduction Using Lithium Aluminium Hydride—I

To LiAlH₄ (2.0 equivalents), in THF at −10° C., was added the nitrile (1.0 equivalent). The mixture was stirred at −10° C. for 30 min. then 0° C. for 30 min. and r.t for 1 hr. The mixture was then cooled to 0° C. and quenched by successive, careful, addition of H₂O (3 equivalents) and 10% aq. NaOH (2 equivalents). After stirring for a further 10 min. the mixture was diluted with THF and filtered. The filtrate was then concentrated in vacuo. and the residue was purified by flash column chromatography on silica gel, eluting with DMAW 90 to afford the desired compound.

### Method F2

### Nitrile Reduction Using Lithium Aluminium Hydride—II

To a solution of the nitrile in organic solvent (typically tetrahydrofuran) at room temperature was added a solution of lithium aluminium hydride in tetrahydrofuran (2 equivalents). The mixture was stirred at room temperature for 1-16 hours then quenched by cautious addition of small amounts of water and sodium hydroxide solution. The reaction was filtered under suction, washing with tetrahydrofuran and methanol then concentrated in vacuo furnishing a crude product that was purified on a silica Biotage column eluting with dichloromethane/methanol or dichloromethane/acetic acid/methanol/water mixtures.

### Method F3

### Nitrile Reduction Using Raney Nickel

A mixture of protected amine and Raney Nickel (typically used was as a suspension in water) in organic solvent (e.g. N,N-dimethylformamide, ethanol and/or tetrahydrofuran), optionally with added base (e.g. aqueous sodium hydroxide solution or methanolic ammonia), was hydrogenated at atmospheric pressure and at room temperature for 18-96 hours. To achieve full reduction, it was occasionally required to refresh the catalyst during this time. When the requisite volume of hydrogen had been consumed, the reaction was filtered under suction using either a celite pad or glass fibre filter paper before concentrating to furnish the desired deprotected amine. This material was ether used crude, or purified by silica column chromatography eluting with mixtures of dichloromethane, methanol, acetic acid and water.

### Method G

### Amide Coupling (EDC, HOBt Method)

To a stirred solution of the acid or sodium salt (1 equivalent) in DMF (10 ml) was added 1-hydroxybenzotriazole (1.2 equivalents), the amine (1-1.2 equivalents) and either diisopropylethylamine or triethylamine (1.2-2.2 eq) followed by N-ethyl-N′-(3-dimethylaminopropyl) carbodiimide hydrochloride (1.2 equivalents). The reaction mixture was either stirred at room temperature or heated at 50-60° C. overnight. The mixture was diluted with ethyl acetate and washed with excess water/aqueous saturated sodium bicarbonate solution, the organic layer was separated and the solvent removed in vacuo to afford the product. The product was either taken on crude or purified by column chromatography on silica (eluting with mixtures of ethyl acetate in petroleum ether).

### Method H1

### Removal of a Carboxybenzyl (Z) Protecting Group By Hydrogenation

A mixture of protected amine and Palladium on carbon (typically 10%, wet) in organic solvent (e.g. ethanol), was hydrogenated at atmospheric pressure and at room temperature for 18-96 hours. To achieve full reduction, it was occasionally required to refresh the catalyst during this time. When the requisite volume of hydrogen had been consumed, the reaction was filtered under suction using either a celite pad or glass fibre filter paper before concentrating to furnish the desired deprotected amine. This material was ether used crude, or purified by silica column chromatography eluting with mixtures of dichloromethane, methanol, acetic acid and water.

### Method H2

Removal of a Carboxybenzyl (Z) Protecting Group By Hydrogenation with In-Situ BOC Protection

The reaction was conducted as described in H1 above, with an excess of di-tert-butyl dicarbonate. Upon work-up, the BOC protected amine was isolated and was optionally purified on silica Biotage column eluting with ethyl acetate/petrol mixtures.

### Method H3

Removal of a Carboxybenzyl (Z) Protecting Group Under Acidic conditions

The protected amine was dissolved in hydrobromic acid in acetic acid (40%) and stirred thus for 1-16 hours. The acids were then removed in vacuo and the residue was optionally re-concentrated from methanol. The crude material was purified on a silica Biotage column eluting with mixtures of dichloromethane, methanol, acetic acid and water.

### Method I

### Alkylation of Amine

To a solution of amine or Z-protected amine in N,N-dimethylformamide cooled to 0° C. was added portionwise sodium hydride (1.5 equivalents). After stirring for 10 minutes, a solution of alkylamine (e.g. iodomethane in tert-butyldimethylether, 1-5 equivalents) was added and the mixture was allowed to warm to room temperature. The crude product was isolated by aqueous extraction and optionally purified on a silica Biotage column.

### Method J

### Nucleophilic Substitution of Halo-Bicyclic Compound by Piperidine Compound Under Microwave Irradiation

A mixture of the piperidine, halobicycle (e.g. 6-chloro-9H-purine), triethylamine (2-10 equivalents) and organic solvent (typically n-butanol or N-methylpyrrolidin-2-one) was irradiated in a sealed microwave vessel to 100-200° C. for 1-5 hours. The reaction was typically filtered under suction washing with suitable organic solvents (e.g. methanol, dichloromethane) then concentrated. Optional aqueous work-up was undertaken followed by purification by silica Biotage column eluting with ethyl acetate/petrol, dichloromethane/acetic acid/methanol/water, or dichloromethane/methanolic ammonia to furnish the pure product.

### Method K

### Carboxybenzyl (Z) Protection

To a solution of the amine in tetrahydrofuran was added an aqueous base in water (e.g. sodium carbonate). The reaction was cooled to 0° C. then benzyl chloroformate was added dropwise. The reaction was left stirring for 6-24 hours, warming slowly to room temperature. The reaction was quenched by addition of water then was extracted with ethyl acetate. The combined organic liquors were washed with brine, dried (MgSO₄) and concentrated to furnish a colourless oil. This crude material was purified on a silica Biotage column eluting with ethyl acetate/petrol mixtures.

### Method YY1

### Amide Coupling

To a mixture of carboxylic acid (1 equivalent), amine (1.1 equivalents), 1-hydroxybenzotriazole (1.1 equivalents) and triethylamine (2.2 equivalents (or 3.3 equivalents if hydrochloride of amine was used)) in N-methylpyrrolidinone was added (N-ethyl-N′-(3-dimethylaminopropyl)carbodiimide hydrochloride (1.1 equivalents). The mixture was then heated at 60° C. with stirring for 16 hours. Upon cooling the reaction mixture was diluted with ethyl acetate and the organic layer was washed with 2M aqueous sodium hydroxide, followed by brine. The organic layer was separated, dried (MgSO₄) and the solvent was removed in vacuo to afford the crude amide intermediate. The product was then either triturated from diethyl ether or was purified by flash column chromatography on silica gel, typically using dichloromethane/methanol as eluent.

### Method YY2

### Boc Deprotection

To the protected amine, optionally dissolved in a suitable organic solvent (typically dichloromethane), was added a strong organic (e.g. trifluoroacetic acid) or inorganic (e.g. hydrochloric acid in 1,4-dioxane) acid. This mixture was stirred at room temperature for between 10 minutes and 18 hours to furnish the crude amine as a salt. If necessary, purification could be achieved via silica column chromatography using a mixture of dichloromethane, methanol, acetic acid and H₂O or dichloromethane, methanol and ammonia, and/or via ion exchange chromatography and/or by preparative HPLC.

### Method YY3

### HCl Salt Formation

The amine (1 equivalent) was dissolved or suspended in methanol and 4M HCl in 1, 4-dioxane was added (1 equivalent). The mixture was stoppered and stirred for 2 hours and was then concentrated in vacuo. The residue was triturated using diethyl ether and the solid was filtered off in vacuo, washing with diethyl ether. The solid was then dried in the vacuum oven.

### Method YY4

### Nitrile Reduction

A solution of 1M lithium aluminium hydride in tetrahydrofuran (2 equivalents) was further diluted with anhydrous tetrahydrofuran and the solution was cooled to 0° C. under nitrogen. The nitrile (1 equivalent) was dissolved in anhydrous tetrahydrofuran and this solution was added dropwise to the solution of lithium aluminium hydride under nitrogen. The resulting reaction mixture was stirred for 30 minutes at 0° C. and then typically 1 hour at room temperature. The reaction mixture was then cooled to 0° C. and was quenched by cautious addition of water, followed by 10% aqueous sodium hydroxide solution, followed by water. The mixture was stirred for 1 hour and was then filtered in vacuo. The filtrate was concentrated in vacuo and was then purified by ion-exchange chromatography followed by silica column chromatography using a dichloromethane/methanol mixture as eluent.

### Examples 14 to 17

By following the methods described above, the compounds of Examples 14 to 17 set out in the Table below are prepared.

| Example | | | |
|---|---|---|---|
| Number | Compound | Chemical Name | Synthetic Procedure |
| 14 | 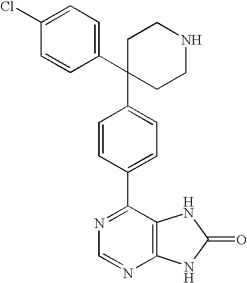 | 6-{4-[4-(4-Chloro- phenyl)-piperidin-4- yl]-phenyl}-7,9- dihydro-purin-8-one | 1. Method A using 4-(4- bromo-phenyl)-4-(4-chloro- phenyl)-piperidine (WO2005/061463 Example 16A) 2. Method B 3. Method C3 4. Method D |
| 15 | 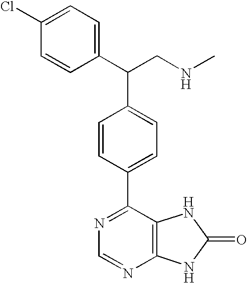 | 6-{4-[1-(4-Chloro- phenyl)-2- methylamino-ethyl]- phenyl}-7,9-dihydro- purin-8-one | 1. Method A using [2-(4- bromo-phenyl)-2-(4-chloro- phenyl)-ethyl]-methyl-amine (WO2005/061463 Example 21) 2. Method B 3. Method C3 4. Method D |
| 16 | 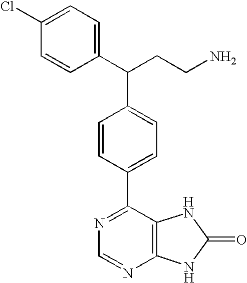 | 6-{4-[3-Amino-1-(4- chloro-phenyl)- propyl]-phenyl}-7,9- dihydro-purin-8-one | 1. Method A using [2-(4- bromo-phenyl)-2-(4-chloro- phenyl)-ethyl]-methyl-amine (WO2005/061463 Example 12B) 2. Method B 3. Method C3 4. Method D |
| 17 | 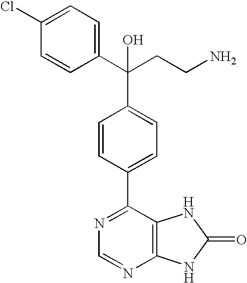 | 6-{4-[3-Amino-1-(4- chloro-phenyl)-1- hydroxy-propyl]- phenyl}-7,9-dihydro- purin-8-one | 1. Method E using (4-chloro- phenyl)-(4-iodo-phenyl)- methanone 2. Method B 3. Method C3 4. Method F |

### Example 18

### 4-Aminomethyl-1-(7H-pyrrolo[2,3-d]pyrimidin-4-yl)-piperidine-4-carboxylic acid (4-chloro-phenyl)-amide

### 18A. 4-(4-Chloro-phenylcarbamoyl)-4-cyano-piperidine-1-carboxylic acid tert-butyl ester

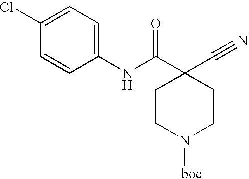

4-Chloroaniline (163 mg, 1.28 mmol) was added to a solution of 4-cyano-piperidine-1,4-dicarboxylic acid mono-tert-butyl ester (250 mg, 0.98 mmol), HATU (486 mg, 1.28 mmol) and Hünig's base (0.86 ml, 4.92 mmol) in DMF (2.5 ml) and stirred at room temperature under an atmosphere of argon. After stirring for 17 h, the reaction mixture was partitioned between dichloromethane and water. The organic layers were then dried, filtered and evaporated. The crude material was purified by flash silica column chromatography, eluting with 25% ethyl acetate-petrol, to afford the title compound (302 mg, 84%)

### 18B. 4-Cyano-piperidine-4-carboxylic acid (4-chloro-phenyl)-amide

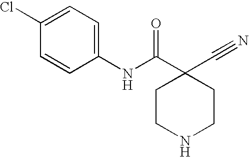

To a solution of 4-(4-chloro-phenylcarbamoyl)-4-cyano-piperidine-1-carboxylic acid tert-butyl ester (302 mg, 0.83 mmol) in methanol (30 ml) at rt was added 4M HCl in dioxane (30 ml). After stirring for 20 h the solution was concentrated to give the deprotected amine as the hydrochloride salt. The crude product was further purified on SCX-II acidic resin, eluting with methanol then 2M ammonia—methanol, to give the title compound (210 mg, 96%).

### 18C. 4-Cyano-1-(7H-pyrrolo[2,3-d]pyrimidin-4-yl)-piperidine-4-carboxylic acid (4-chloro-phenyl)-amide

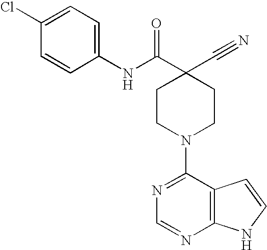

A degassed mixture of 4-cyano-piperidine-4-carboxylic acid (4-chloro-phenyl)-amide (210 mg, 0.80 mmol), 4-chloro-7H-pyrrolo[2,3-d]pyrimidine (122 mg, 0.80 mmol), triethylamine (777 μL, 5.57 mmol) and n-butanol (1.5 mL) was heated to 100° C. for 1.5 h in a microwave. The reaction mixture was then partitioned between ethyl acetate and saturated aqueous ammonium chloride solution. The organic layer was dried, filtered and concentrated. The crude mixture was then purified by flash silica column chromatography, eluting with 10% methanol-dichloromethane to afford the title compound (142 mg, 47%)

### 18D. 4-Aminomethyl-1-(7H-pyrrolo[2,3-d]pyrimidin-4-yl)-piperidine-4-carboxylic acid (4-chloro-phenyl)-amide

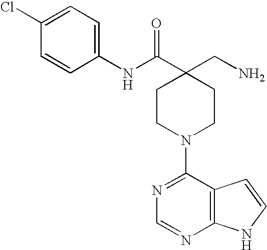

Sodium borohydride (141 mg, 3.73 mmol) was added portionwise, slowly, to a stirred solution of 4-cyano-1-(7H-pyrrolo[2,3-d]pyrimidin-4-yl)-piperidine-4-carboxylic acid (4-chloro-phenyl)-amide (142 mg, 0.37 mmol) and NiCl₂.6H₂O (177 mg, 0.75 mmol) in methanol (3 ml) at 0° C. After 15 minutes, the reaction mixture was allowed to warm to room temperature and stirred for a further 17 hours. The reaction mixture was diluted with methanol and concentrated HCl added (37.3 mmol). The mixture was then heated to reflux for 1 hour. Upon cooling, the solvent was removed in vacuo and the residue purified by flash silica column chromatography, eluting with 10% 2M ammonia in methanol—dichloromethane to afford the title compound (40 mg, 28%). LC-MS m/z 385. ¹H NMR (400 MHz, Me-d3-OD): 8.14 (1H, s), 7.60 (2H, d), 7.33 (2H, d), 7.14 (1H, d), 6.65 (1H, d), 4.44-4.34 (2H, m), 3.77-3.65 (2H, m), 2.96 (2H, s), 2.35 (2H, d), 1.76-1.65 (2H, m).

### Example 19

### Example B

### 4-Aminomethyl-1-(7Hpyrrolo[2,3-d]pyrimidin-4-yl)-piperidine-4-carboxylic acid 4-chloro-benzylamide

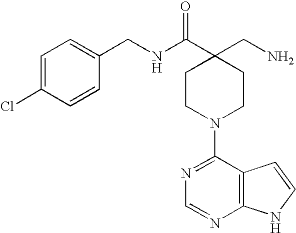

The title compound was prepared as described for Example 18 (A) but substituting 4-chloroaniline for 4-chlorobenzylamine. LC-MS m/z 399. ¹H NMR (400 MHz, DMSO-d6): 8.60 (1H, t), 8.12 (1H, s), 7.37 (2H, d), 7.31 (2H, d), 7.16 (1H, t), 6.57 (1H, d), 4.32 (2H, d), 4.30-4.21 (2H, m), 3.50-3.45 (2H, m), 2.68 (2H, s), 2.09 (2H, d), 1.54-1.43 (2H, m).

### Example 20

### Example D

### 4-Amino-1-(9H-purin-6-yl)-piperidine-4-carboxylic acid 4-chloro-benzylamide

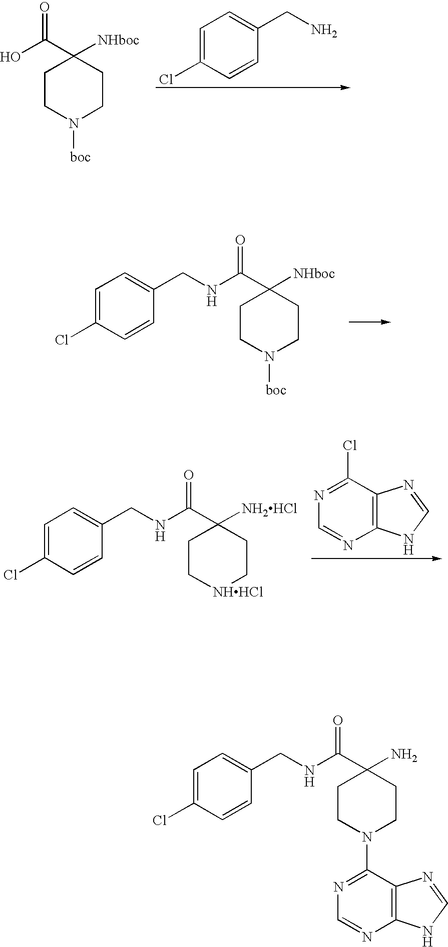

### 20A. 4-tert-Butoxycarbonylamino-4-(4-chloro-benzylcarbamoyl)-piperidine-1-carboxylic acid tert-butyl ester

Dry DMF (1 mL) was added to a mixture of 4-tert-butoxycarbonylamino-piperidine-1,4-dicarboxylic acid mono tert-butyl ester (151 mg, 0.44 mmol) and HATU (220 mg, 0.58 mmol) under nitrogen. N-Ethyldiisopropylamine (0.38 mL, 2.1 mmol) was added to the solution and the reaction mixture was stirred for 15 min. 4-Chlorobenzylamine (70 μL, 0.57 mmol) was added and the solution was stirred for 23 hours at room temperature and under nitrogen. The reaction mixture was partioned between dichloromethane (10 mL) and water (10 mL). The aqueous phase was further extracted with dichloromethane (20 mL). The combined organic layers were dried (Mg₂SO₄), filtered and concentrated. Flash column chromatography on silica, eluting with 4% methanol in dichloromethane, gave 4-tert-butoxycarbonylamino-4-(4-chloro-benzylcarbamoyl)-piperidine-1-carboxylic acid tert-butyl ester (177 mg, 0.38 mmol, 86%). LC-MS (LCT2) m/z 490 [M+Na⁺], Rₜ 8.09 min.

### 20B. 4-Amino-piperidine-4-carboxylic acid 4-chloro-benzylamide dihydrochloride

A 4M solution of HCl in dioxane (7.7 ml, 31 mmol) was added dropwise to a solution of 4-tert-butoxycarbonylamino-4-(4-chloro-benzylcarbamoyl)-piperidine-1-carboxylic acid tert-butyl ester (96 mg, 0.20 mmol) in methanol (7.7 mL) and stirred at rt for 17 h. The solvents were concentrated to give 4-amino-piperidine-4-carboxylic acid 4-chloro-benzylamide dihydrochloride (71 mg, 0.20 mmol, 100%) that was used in the next step without further purification.

¹H NMR (500 MHz, CD₃OD): 2.18 (2H, m), 2.64 (2H, m), 3.44 (4H, m), 4.47 (2H, s), 7.36 (4H, m).

### 20C. 4-Amino-1-(9H-purin-6-yl)-piperidine-4-carboxylic acid 4-chloro-benzylamide

A degassed mixture of 4-amino-piperidine-4-carboxylic acid 4-chloro-benzylamide dihydrochloride (48 mg, 0.13 mmol), 6-chloropurine (0.12 mmol), triethylamine (126 μL, 0.9 mmol) and n-butanol (1.2 mL) was stirred at 100° C. for 18 hours. The solvents were removed by evaporation and the crude mixture was first purified on a SCX-II acid resin, eluting with methanol then 2M ammonia/methanol, and then by preparative TLC, eluting with 10% methanol in dichloromethane, to give the title compound. LC-MS m/z 386. ¹H NMR (400 MHz, Me-d3-OD): 8.22 (1H, s), 8.01 (1H, s), 7.32 (2H, d), 7.29 (2H, d), 5.06 (2H, s), 4.39 (2H, s), 3.76 (2H, t), 2.27-2.14 (2H, m), 1.61 (2H, d).

### Example 21

### 4-Amino-1-(6-oxo-6,7-dihydro-5H-pyrrolo[2,3-d]pyrimidin-4-yl)-piperidine-4-carboxylic acid 4-chloro-benzylamide

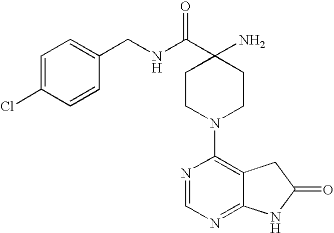

The title compound was prepared as described Example 20 but using 4-chloro-5,7-dihydro-pyrrolo[2,3-d]pyrimidin-6-one (see Example 1D) in place of 6-chloropurine. LC-MS m/z 401. ¹H NMR (400 MHz, Me-d3-OD): 8.19 (1H, s), 7.33 (2H, d), 7.29 (2H, d), 4.38 (2H, s), 4.29-4.18 (2H, m), 3.76 (2H, s), 3.58-3.46 (2H, m), 2.20-2.08 (2H, m), 1.54 (2H, d).

### Example 22

### [4-Amino-1-(7H-pyrrolo[2,3-d]pyrimidin-4-yl)-piperidin-4-yl]-(2,3-dihydro-indol-1-yl)-methanone

### 22A. 4-tert-Butoxycarbonylamino-4-(2,3-dihydro-indole-1-carbonyl)-piperidine-1-carboxylic acid tert-butyl ester

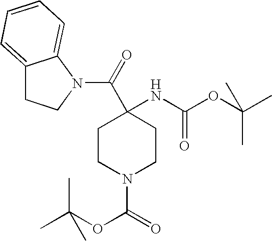

indoline (195 μl, 1.74 mmol) was added to a stirred solution of 4-tert-Butoxycarbonylamino-piperidine-1,4-dicarboxylic acid mono-tert-butyl ester (600 mg, 1.74 mmol), HATU (861 mg, 2.26 mmol) and Hünig's base (1.52 ml, 8.71 mmol) in DMF (5 ml) and stirred at room temperature under an atmosphere of argon. After stirring for 17 h, the reaction mixture was partitioned between dichloromethane and water. The organic layers were then dried, filtered and evaporated. The crude material was purified by flash silica column chromatography, eluting with 25% ethyl acetate-petrol, to afford the title compound (242 mg, 31%)

### 22B. (4-Amino-piperidin-4-yl)-(2,3-dihydro-indol-1-yl)-methanone

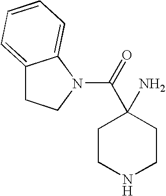

The title compound was prepared according to the method described in Example 1F.

### 22C. [4-Amino-1-(7H-pyrrolo[2,3-d]pyrimidin-4-yl)-piperidin-4-yl]-(2,3-dihydro-indol-1-yl)-methanone

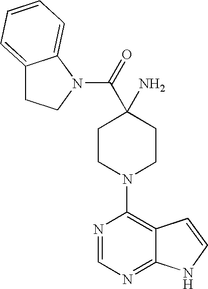

The title compound was prepared according to the method described in Example 2G. LC-MS m/z 363. ¹H NMR (400 MHz, Me-d3-OD): 8.18-8.11 (2H, m), 7.26 (1H, d), 7.17 (1H, d), 7.14 (2H, d), 7.08-7.02 (1H, m), 6.68 (1H, d), 4.60 (2H, t), 4.25-4.16 (2H, m), 4.06-3.99 (2H, m), 3.17-3.11 (2H, m), 2.44-2.37 (2H, m), 1.92-1.83 (2H, m).

### Example 23

### 4-Amino-1-(7H-pyrrolo[2,3-d]pyrimidin-4-yl)-piperidine-4-carboxylic acid 4-methoxy-benzylamide

### 23A 4-tert-Butoxycarbonylamino-4-(4-methoxy-benzylcarbamoyl)-piperidine-1-carboxylic acid tert-butyl ester

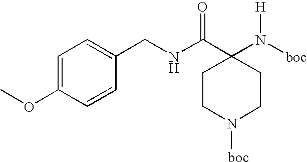

4-Methoxybenzylamine (0.55 g, 4 mmol) 4-tert-butoxycarbonylamino-piperidine-1,4-dicarboxylic acid mono-tert-butyl ester compound (1.38 g, 4 mmol), HOBT (0.648 g 4.8 mmol) and EDC (0.92 g, 4.8 mmol) in DMF (20 mls) were stirred at room temperature for 18 h. The reaction mixture was partitioned between dichloromethane and water. The organic layers were then dried, filtered and evaporated. The crude material was purified by flash silica column chromatography, eluting with petroleum ether/ethyl acetate gradient, to afford the title compound (1.4 g, 75%).

### 23B 4-Amino-piperidine-4-carboxylic acid 4-methoxy-benzylamide

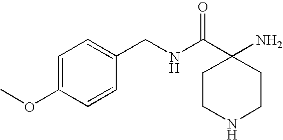

4-tert-Butoxycarbonylamino-4-(4-methoxy-benzylcarbamoyl)-piperidine-1-carboxylic acid tert-butyl ester (1.4 g, 3 mM) was dissolved in dichloromethane (30 mls) and trifluoroacetic acid (15 ml). The reaction mixture was stirred at room temperature for 2 h. The solvent was evaporated and the residue loaded onto a 10 g SCX cartridge. The cartridge was eluted with methanol then 2M ammonia in methanol. The methanolic ammonia solution was evaporated under reduced pressure to give the title compound (0.75 g, 95%).

### 23C. 4-Amino-1-(7H-pyrrolo[2,3-d]pyrimidin-4-yl)-piperidine-4-carboxylic acid 4-methoxy-benzylamide

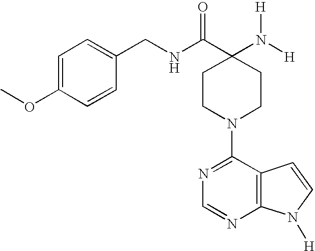

4-Amino-piperidine-4-carboxylic acid 4-methoxy-benzylamide (168 mgs, 0.5 mmol) and 6-chlorodeazapurine (76 mgs, 0.5 mmol) in n-butanol (10 mls) with triethylamine (0.28 mls 4 eq) were heated at 120° C. for 66 h. The solvent was evaporated and the residue loaded onto a 10 g SCX cartridge. The cartridge was eluted with methanol then 2M ammonia in methanol. The methanolic ammonia solution was evaporated under reduced pressure to give an oil. The oil was triturated with acetonitrile, the solid obtained collected by filtration to give the title compound (120 mgs, 63%). LC-MS m/z 380. ¹H NMR (400 MHz, DMSO-d6): 11.65 (1H, s), 8.45 (1H, t), 8.13 (1H, s), 7.22-7.12 (4H, m), 6.87 (2H, d), 6.59 (1H, dd), 4.41 (2H, d), 4.21 (2H, d), 3.73 (3H, s), 3.53 (2H, t), 2.26 (1H, s), 2.05-1.92 (2H, m), 1.44 (2H, d).

### Preparation of Intermediate Compounds A-G and P

### Preparation A

### 5-Bromo-4′-cyano-3′,4′,5′,6′-tetrahydro-2′H-[3,4′]bipyridinyl-1′-carboxylic acid tert-butyl ester

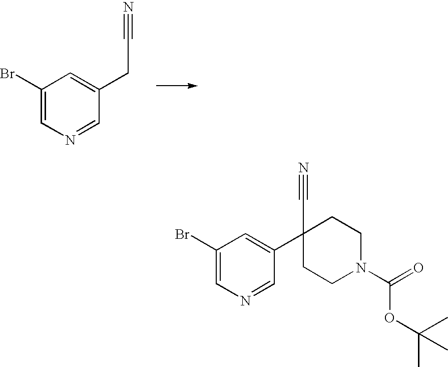

To a solution of (5-bromo-pyridin-3-yl)-acetonitrile (3.62 g, 18.4 mmol) and bis-(2-chloro-ethyl)-carbamic acid tert-butyl ester (made using a method described in J. Chem. Soc., Perkin Trans 1, 2000, p3444-3450. 4.05 g, 16.7 mmol) in dry N,N-dimethylformamide (15 ml) at room temperature was added sodium hydride (1.53 g, 38.4 mmol). The mixture was heated to 60° C. under nitrogen. After 3 hours an additional 8 ml N,N-dimethylformamide was added. After a further 3 hours the reaction was allowed to cool then water was added and the reaction was extracted with ethyl acetate (×3). The organic liquors were combined and washed with brine, dried (MgSO₄) and concentrated in vacuo. The crude product was purified on a silica Biotage column, eluting 40-65% diethyl ether/petrol furnishing the title compound as a yellow oil (3.55 g, 53%).

### Preparation E

### 4-Fluoro-1,3-dihydro-pyrrolo[2,3-b]pyridin-2-one

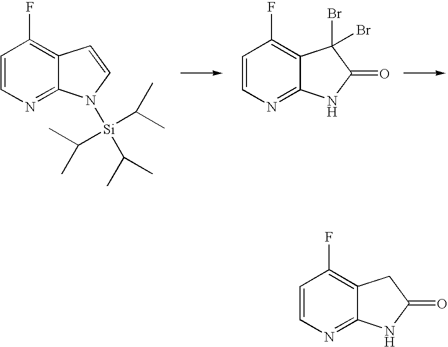

### E1. 3,3-Dibromo-4-fluoro-1,3-dihydro-pyrrolo[2,3-b]pyridin-2-one

To a solution of 4-fluoro-1-triisopropylsilanyl-1H-pyrrolo[2,3-b]pyridine (Org Lett 2003, 5, 5023-5026, 1.0 g, 3.4 mmol) in tert-butanol (25 ml) was added portionwise pyridine tribromide (3.8 g, 11.97 mmol) and this mixture was stirred at room temperature for 3 days. The solvent was removed in vacuo, water and ethyl acetate was added. the mixture was filtered under suction then the organic layer separated. The aqueous fraction was extracted twice with ethyl acetate then the organic liquors were combined and concentrated. The crude product was purified on a silica Biotage column, eluting with petrol/ethyl acetate to furnish the clean product (312 mg, 29%).

### E2. 4-Fluoro-1,3-dihydro-pyrrolo[2,3-b]pyridin-2-one

A mixture of 3,3-dibromo-4-fluoro-1,3-dihydro-pyrrolo[2,3-b]pyridin-2-one (312 mg, 1.0 mmol), acetic acid (4.5 ml), zinc dust (658 mg, 10 mmol) and methanol (4.5 ml) was stirred at room temperature for 3 hours. Brine was added and the reaction was extracted with ethyl acetate. The organic liquors were washed with brine, dried (MgSO₄) and concentrated to furnish the title compound (184 mg, contains ˜40% des-fluorinated product). Used thus in further reactions.

### Preparation F

### 4-Chloro-5,7-dihydro-pyrrolo[2,3-d]pyrimidin-6-one

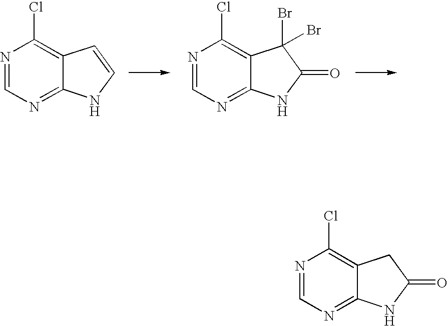

Prepared according to the protocols in Preparation E using 4-chloro-7H-pyrrolo[2,3-d]pyrimidine.

### Preparation G

### C-[4-(3-Chloro-phenyl)-1-(9H-purin-6-yl)-piperidin-4-yl]-methylamine

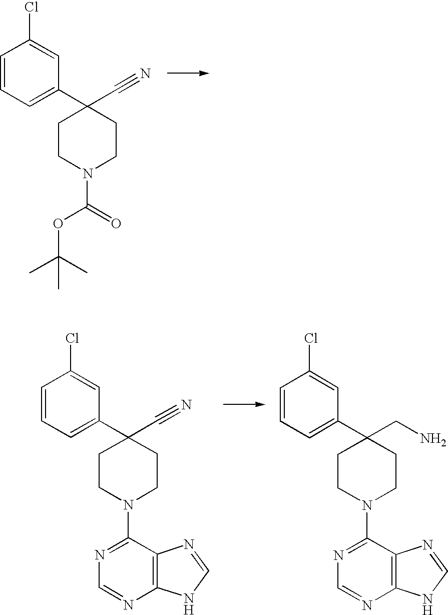

### G1. 4-(3-Chloro-phenyl)-1-(9H-purin-6-yl)-piperidine-4-carbonitrile

To a solution of 4-(3-chloro-phenyl)-4-cyano-piperidine-1-carboxylic acid tert-butyl ester (965 mg, 3.0 mmol) in dichloromethane (10 ml) was added trifluoroacetic acid (4 ml). This mixture was stirred at room temperature for 30 minutes then concentrated in vacuo and re-concentrated from methanol (×2). To this oil was added 6-chloro-9H-purine (464 mg, 3.0 mmol), triethylamine (1.0 ml) and n-butanol (5 ml) then the mixture was heated to 160° C. in a sealed tube in the microwave for 3 hours. The reaction was concentrated in vacuo, triturated with methanol and the solid was dried in a vacuum oven (672 mg, 66%).

### G2. C-[4-(3-Chloro-phenyl)-1-(9H-purin-6-yl)-piperidin-4-yl]-methylamine

To a solution of 4-(3-chloro-phenyl)-1-(9H-purin-6-yl)-piperidine-4-carbonitrile (672 mg, 1.98 mmol) in tetrahydrofuran (20 ml) at room temperature under nitrogen was added lithium aluminium hydride (1M in tetrahydrofuran, 3.97 ml, 4 mmol). A precipitate formed so an additional 20 ml solvent was added. After stirring thus overnight, the reaction was quenched with water (200 μl), sodium hydroxide solution (15%, 200 μl) and then water (600 μl). This mixture was stirred for 30 minutes then the reaction was concentrated in vacuo. The residue was wet with methanol and was filtered under suction. The organic liquors were purified on silica Biotage column eluting DMAW120 to DMAW90. This material was purified by preparative HPLC then re-purified on a second Biotage column to furnish a white solid (131 mg, 19%).

### Preparation P

### 4-tert-butoxycarbonylamino-1-(7H-pyrrolo[2,3-d]pyrimidin-4-yl)-piperidine-4-carboxylic acid

### P1. 4-tert-butoxycarbonylamino-1-(7H-pyrrolo[2,3-d]pyrimidin-4-yl)-piperidine-4-carboxylic acid ethyl ester

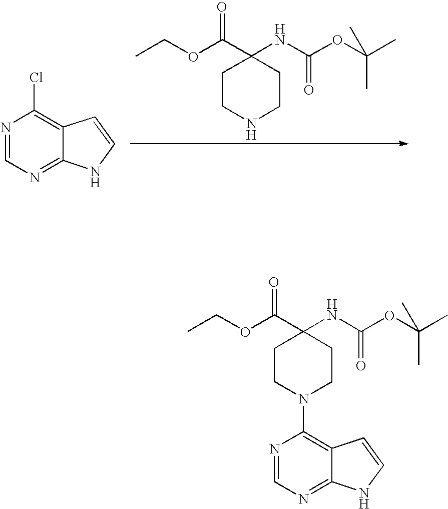

4-tert-butoxycarbonylamino-piperidine-4-carboxylic acid ethyl ester (5 g, 19.4 mmol*) was dissolved in N-methylpyrrolidinone (41 mL) and triethylamine (2.9 mL, 21.3 mmol) was added followed by 4-chloro-7H-pyrrolo[2,3-d]pyrimidine (3.27 g, 21.3 mmol). The resulting mixture was heated at 110° C. under nitrogen for 4 hours. The reaction mixture was allowed to stand for 64 hours. The reaction mixture was diluted with ethyl acetate and the organic was washed three times with water. The organic was separated off, dried (MgSO₄) and concentrated in vacuo. The residue was purified by flash column chromatography on silica gel, eluting with 50/50 ethyl acetate/petroleum ether to afford the title compound as a yellow oil (9.70 g, >100%).

* Commercially available from Astatech (catalogue number: 55743)

### P2. 4-tert-butoxycarbonylamino-1-(7H-pyrrolo[2,3-d]pyrimidin-4-yl)-piperidine-4-carboxylic acid

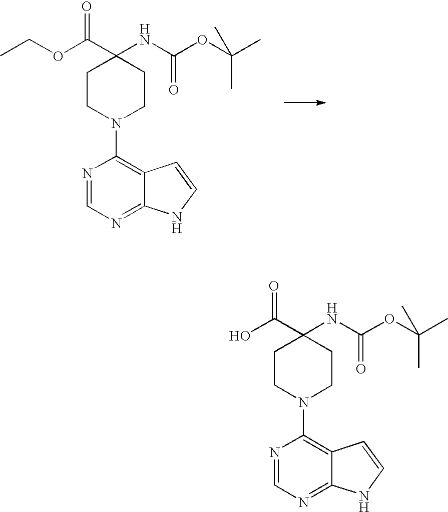

4-tert-butoxycarbonylamino-1-(7H-pyrrolo[2,3-d]pyrimidin-4-yl)-piperidine-4-carboxylic acid ethyl ester (7.28 g, 19.4 mmol) was dissolved in a 1:1 mixture of ethanol and tetrahydrofuran (100 mL in total). A solution of sodium hydroxide (3.88 g, 97 mmol) in water (50 mL) was made up and this was added to the solution of 4-tert-butoxycarbonylamino-1-(7H-pyrrolo[2,3-d]pyrimidin-4-yl)-piperidine-4-carboxylic acid ethyl ester. The resulting mixture was stirred at 60° C. for 18 hours. The reaction was allowed to cool and was concentrated in vacuo. The residue was dissolved in water (100 mL) and was acidified cautiously with conc. HCl to pH 4-5 with ice-cooling. The aqueous was extracted four times with ethyl acetate, each time ensuring an aqueous pH of 4-5. The organics were combined, dried (MgSO₄) and concentrated in vacuo to afford the title compound as a yellow gum (7.3 g, >100%). The product was used without further purification.

### Examples 24 to 43

By using the methods and the intermediates described above, the compounds of Examples 24 to 43 were prepared.

| Example | | | | | |
|---|---|---|---|---|---|
| Number | Compound | Chemical Name | Method | N.M.R. Data | M.S. |
| 24 | 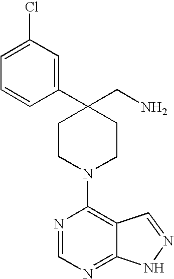 | C-[4-(3-Chloro- phenyl)-1-(1H- pyrazolo[3,4- d]pyrimidin-4- yl)-piperidin-4- yl]-methylamine (diacetate salt) | As for Preparation G | ¹H NMR (400 MHz, Me-d₃-OD): 8.29-8.25 (1 H, m), 8.24 (1 H, s), 7.61 (1 H, s), 7.55-7.47 (2 H, m), 7.45-7.38 (1 H, m), 4.51 (2 H, m), 3.54 (2 H, m), 3.12 (2 H, s), 2.51 (2 H, m), 2.01 (2 H, m), 1.98 (6 H, m) | M/z: 343 |
| 25 | 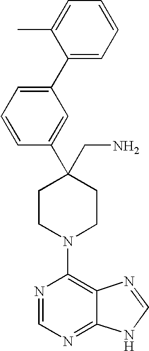 | C-[4-(2′-Methyl- biphenyl-3-yl)- 1-(9H-purin-6- yl)-piperidin-4- yl]-methylamine (diacetate salt) | Method C1 using Preparation G & o-tolylboronic acid | ¹H NMR (Me-d₃- OD) 8.22 (1 H, s), 8.03 (1 H, s), 7.59 (2 H, m), 7.51 (1 H, s), 7.35 (1 H, d), 7.28 (4 H, m), 5.02 (2 H, m), 3.61 (2 H, m), 3.20 (2 H, s), 2.52 (2 H, m), 2.30 (3 H, s), 2.01 (2 H, m), 1.98 (6 H, s) | M/z: 399 |
| 26 | 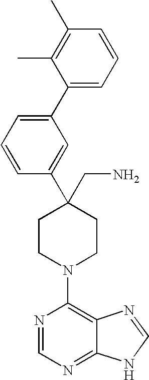 | C-[4-(2′,3′- Dimethyl- biphenyl-3-yl)- 1-(9H-purin-6- yl)-piperidin-4- yl]-methylamine (diacetate salt) | Method C1 using Preparation G & 2,3- dimethylphenyl- boronic acid | ¹H NMR (400 MHz, Me-d₃-OD): 8.23 (1 H, s), 8.04 (1 H, s), 7.64-7.53 (2 H, m), 7.47 (1 H, s), 7.33 (1 H, d), 7.23-7.11 (2 H, m), 7.09 (1 H, d), 5.03 (2 H, d), 3.60 (2 H, t), 3.26-3.15 (2 H, m), 2.54 (2 H, d), 2.36 (3 H, s), 2.19 (3 H, s), 2.07-1.91 (8 H, m). | M/z: 413 |
| 27 | 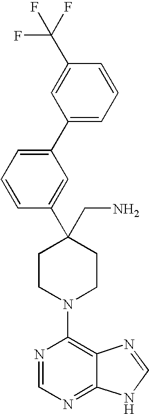 | C-[1-(9H-Purin- 6-yl)-4-(3′- trifluoromethyl- biphenyl-3-yl)- piperidin-4-yl]- methylamine | Method C1 using Preparation G & 3- trifluoromethyl- phenyl boronic acid | ¹H NMR (DMSO- d₆) 8.19 (1 H, s), 8.11 (1 H, s), 8.02 (1 H, d), 7.99 (1 H, s), 7.71 (3 H, m), 7.60 (1 H, m), 7.50 (2 H, m), 4.70 (2 H, m), 3.70 (2 H, m), 2.76 (2 H, s), 2.28 (2 H, m), 1.93 (2 H, m) | M/z: 453 |
| 28 | 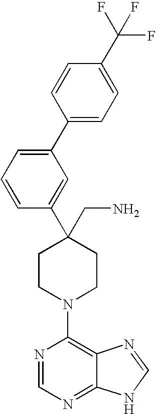 | C-[1-(9H-Purin- 6-yl)-4-(4′- trifluoromethyl- biphenyl-3-yl)- piperidin-4-yl]- methylamine | Method C1 using Preparation G & 4-trifluoro- methyl-phenyl boronic acid | ¹H NMR (DMSO- d₆) 8.20 (1 H, s), 8.10 (1 H, s), 7.92 (2 H, d), 7.81 (2 H, d), 7.71 (1 H, s), 7.60 (1 H, m), 7.52 (2 H, m), 4.72 (2 H, m), 3.70 (2 H, m), 2.75 (2 H, s), 2.28 (2 H, m), 1.92 (2 H, m) | M/z: 453 |
| 29 | 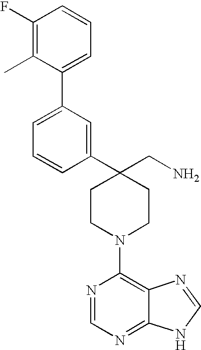 | C-[4-(3′-Fluoro- 2′-methyl- biphenyl-3-yl)- 1-(9H-purin-6- yl)-piperidin-4- yl]-methylamine (bis- trifluoroacetate) | 1. Method C1 using Preparation G & 3-fluoro-2- methylbenzene boronic acid 2. Method A 3. Method D | ¹H NMR (400 MHz, DMSO-d₆): 8.44 (1 H, s), 8.27 (1 H, s), 7.69-7.57 (2 H, m), 7.52 (1 H, s), 7.40 (1 H, d), 7.34-7.22 (1 H, m), 7.16-7.04 (2 H, m), 5.02 (2 H, m), 3.80 (2 H, m), 3.30 (2 H, s), 2.70 (2 H, m), 2.27 (3 H, s), 2.12 (2 H, m) | M/z: 417 |
| 30 | 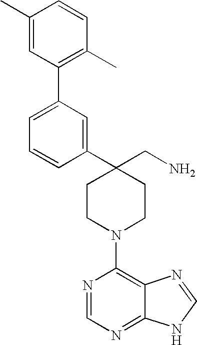 | C-[4-(2′,5′- Dimethyl- biphenyl-3-yl)- 1-(9H-purin-6- yl)-piperidin-4- yl]-methylamine (diacetate salt) | Method C1 using Preparation G & 2,5-dimethyl- benzene boronic acid | ¹H NMR (Me-d₃- OD) 8.23 (1 H, s), 8.02 (1 H, s), 7.59 (2 H, m), 7.48 (1 H, s), 7.32 (1 H, d), 7.18 (1 H, d), 7.08 (2 H, m), 5.01 (2 H, m), 3.61 (2 H, m), 3.20 (2 H, s), 2.52 (2 H, m), 2.35 (3 H, s), 2.24 (3 H, s), 2.00 (8 H, m) | M/z: 413 |
| 31 | 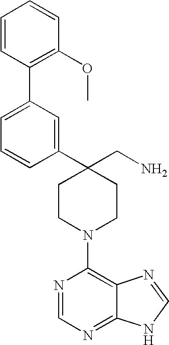 | C-[4-(2′- Methoxy- biphenyl-3-yl)- 1-(9H-purin-6- yl)-piperidin-4- yl]-methylamine | Method C1 using Preparation G & 2-methoxy- benzene boronic acid | ¹H NMR (Me-d₃- OD) 8.20 (1 H, s), 8.01 (1 H, s), 7.63 (1 H, s), 7.48 (2 H, m), 7.41 (1 H, d), 7.33 (2 H, m), 7.10 (1 H, d), 7.03 (1 H, t), 4.98 (2 H, m), 3.82 (3 H, s), 3.62 (2 H, m), 2.82 (2 H, s), 2.46 (2 H, m), 1.88 (2 H, m) | M/z: 415 |
| 32 | 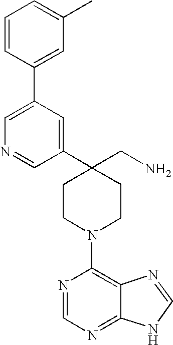 | C-[1′-(9H-Purin- 6-yl)-5-m-tolyl- 2′,3′,5′,6′- tetrahydro-1′H- [3,4′]bipyridinyl- 4′-yl]- methylamine (acetate salt) | 1. Method C2 using Preparation A & 3-tolylboronic acid 2. Method H1 3. Method K 4. Method D 5. Method J using 6-chloro- 9H-purine 6. Method H2 7. Method D | ¹H NMR (400 MHz, MeOD): 8.91 (1 H, d), 8.79 (1 H, d), 8.38 (1 H, s), 8.32 (1 H, s), 8.14 (1 H, s), 7.61 (1 H, s), 7.56 (1 H, d), 7.45 (1 H, t), 7.34 (1 H, d), 3.93- 3.79 (2 H, m), 3.43 (2 H, s), 2.70-2.58 (2 H, m), 2.48 (3 H, s), 2.24-2.11 (2 H, m), 2.05-1.96 (5 H, m). | M/z: 400 |
| 33 | 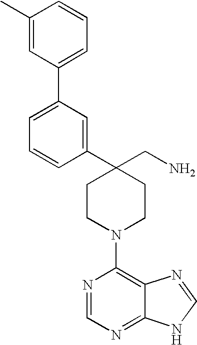 | C-[4-(3′-Methyl- biphenyl-3-yl)- 1-(9H-purin-6- yl)-piperidin-4- yl]-methylamine | 1. Method C1 using 3- tolylboronic acid & 4-(3-bromo- phenyl)-4- cyano- piperidine-1- carboxylic acid tert-butyl ester 2. Method D 3. Method J using 6-chloro- 9-(tetrahydro- pyran-2-yl)-9H- purine (JACS 1961 (83) 2574- 2579) 4. Method F2 | ¹H NMR (400 MHz, Me-d₃-OD): 8.19 (1 H, s), 8.00 (1 H, s), 7.67 (1 H, s), 7.58-7.50 (2 H, m), 7.50-7.44 (2 H, m), 7.42 (1 H, d), 7.33 (1 H, t), 7.18 (1 H, d), 4.90 (2 H, s), 3.74-3.55 (2 H, m), 2.85 (2 H, s), 2.47 (2 H, d), 2.42 (3 H, s), 2.09-1.86 (2 H, m). | M/z: 399 |
| 34 | 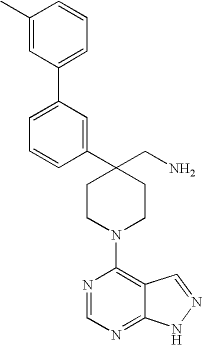 | C-[4-(3′-Methyl- biphenyl-3-yl)- 1-(1H- pyrazolo[3,4- d]pyrimidin-4- yl)-piperidin-4- yl]-methylamine | 1. Method C1 using 3- tolylboronic acid & 4-(3-bromo- phenyl)-4- cyano- piperidine-1- carboxylic acid tert-butyl ester 2. Method D 3. Method J using 4-chloro- 1H- pyrazolo[3,4- d]pyrimidine 4. Method F2 | ¹H NMR (400 MHz, Me-d₃-OD): 8.25 (2 H, d), 7.70 (1 H, s), 7.59-7.39 (5 H, m), 7.35 (1 H, t), 7.20 (1 H, d), 4.56-4.44 (2 H, m), 3.60 (2 H, t), 2.90 (2 H, s), 2.55 (2 H, d), 2.44 (3 H, s), 2.05-1.93 (2 H, m). | M/z: 399 |
| 35 | 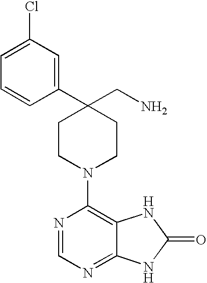 | 6-[4- Aminomethyl-4- (3-chloro- phenyl)- piperidin-1-yl]- 7,9-dihydro- purin-8-one | 1. Method D using 4-(3- chloro-phenyl)- 4-cyano- piperidine-1- carboxylic acid tert-butyl ester 2. Method J using 6-chloro- 7,9-dihydro- purin-8-one 3. Method F2 | ¹H NMR (400 MHz, DMSO-d₆): 8.09-8.03 (1 H, m), 7.44-7.33 (3 H, m), 7.33-7.26 (1 H, m), 3.86-3.75 (2 H, m), 3.26-3.14 (2 H, m), 2.70 (2 H, s), 2.12 (2 H, d), 1.90-1.79 (2 H, m). | M/z: 359 |
| 36 | 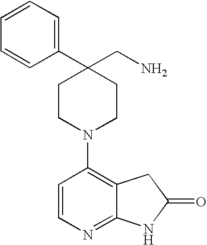 | 4-(4- Aminomethyl-4- phenyl- piperidin-1-yl)- 1,3-dihydro- pyrrolo[2,3- b]pyridin-2-one | 1. Method F3 using 4-(3- chloro-phenyl)- 4-cyano- piperidine-1- carboxylic acid tert-butyl ester 2. Method K 3. Method D 4. Method J using Preparation E 5. Method H1 | ¹H NMR (400 MHz, DMSO-d₆): 7.73 (1 H, d), 7.37 (5 H, d), 7.30-7.17 (1 H, m), 6.42 (1 H, d), 3.61 (2 H, s), 3.57-3.47 (2 H, m), 3.04 (2 H, t), 2.65 (2 H, s), 2.14 (2 H, d), 1.93-1.80 (2 H, m). | M/z: 323 |
| 37 | 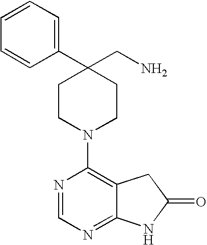 | 4-(4- Aminomethyl-4- phenyl- piperidin-1-yl)- 5,7-dihydro- pyrrolo[2,3- d]pyrimidin-6- one | 1. Method F3 using 4-(3- chloro-phenyl)- 4-cyano- piperidine-1- carboxylic acid tert-butyl ester 2. Method K 3. Method D 4. Method J using Preparation F 5. Method H1 | 1H NMR (400 MHz, DMSO-d₆): 8.15 (1 H, s), 7.43- 7.29 (5 H, m), 7.29- 7.18 (1 H, m), 4.02- 3.90 (2 H, m), 3.71 (2 H, s), 3.28-3.17 (2 H, m), 2.64 (2 H, s), 2.11 (2 H, d), 1.87-1.74 (2 H, m). | M/z: 325 |
| 38 | 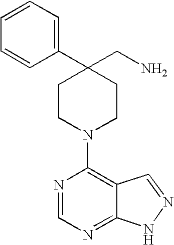 | C-[4-Phenyl-1- (1H- pyrazolo[3,4- d]pyrimidin-4- yl)-piperidin-4- yl]-methylamine (bis-acetate) | 1. Method F3 using 4-(3- chloro-phenyl)- 4-cyano- piperidine-1- carboxylic acid tert-butyl ester 2. Method K 3. Method D 4. Method J using 4-chloro- 1H- pyrazolo[3,4- d]pyrimidine 5. Method H1 | ¹H NMR (400 MHz, Me-d₃-OD): 8.25 (2 H, d), 7.63- 7.48 (4 H, m), 7.40 (1 H, t), 4.55 (2 H, d), 3.57-3.42 (2 H, m), 3.15 (2 H, s), 2.56 (2 H, d), 2.06- 1.98 (2 H, m). | M/z: 309 |
| 39 | 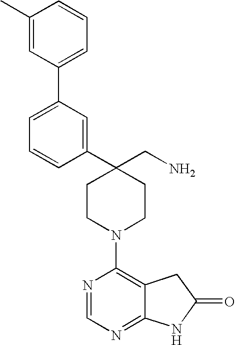 | 4-[4- Aminomethyl-4- (3′-methyl- biphenyl-3-yl)- piperidin-1-yl]- 5,7-dihydro- pyrrolo[2,3- d]pyrimidin-6- one | 1. Method C1 using 4-(3- bromo-phenyl)- 4-cyano- piperidine-1- carboxylic acid tert-butyl ester & 3-tolylboronic acid 2. Method F3 3. Method K 4. Method D 5. Method J using Preparation F 6. Method H1 | ¹H NMR (400 MHz, DMSO-d₆): 8.25 (1 H, s), 7.68 (1 H, s), 7.63-7.51 (4 H, m), 7.51-7.41 (2 H, m), 7.28 (1 H, d), 4.11-4.01 (2 H, m), 3.82 (2 H, s), 2.84 (2 H, s), 2.48 (3 H, s), 2.29 (2 H, d), 2.02-1.90 (2 H, m). | M/z: 414 |
| 40 | 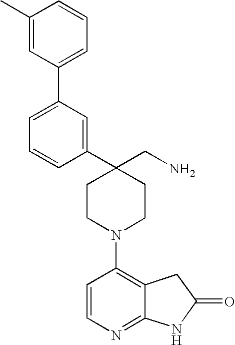 | 4-[4- Aminomethyl-4- (3′-methyl- biphenyl-3-yl)- piperidin-1-yl]- 1,3-dihydro- pyrrolo[2,3- b]pyridin-2-one (bis-acetate) | 1. Method C1 using 4-(3- bromo-phenyl)- 4-cyano- piperidine-1- carboxylic acid tert-butyl ester & 3-tolylboronic acid 2. Method F3 3. Method K 4. Method D 5. Method J using Preparation E 6. Method H1 | ¹H NMR (400 MHz, DMSO-d₆): 7.73 (1 H, d), 7.58 (1 H, s), 7.53-7.42 (4 H, m), 7.42-7.32 (2 H, m), 7.19 (1 H, d), 6.44 (1 H, d), 3.62 (2 H, s), 3.61- 3.52 (2 H, m), 3.13- 3.04 (2 H, m), 2.72 (2 H, s), 2.39 (3 H, s), 2.23 (2 H, d), 1.98-1.90 (2 H, m), 1.88 (6 H, s). | M/z: 413 |
| 41 | 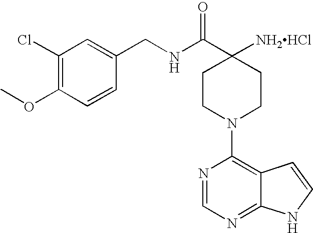 | 4-amino-1-(7H- pyrrolo[2,3- d]pyrimidin-4- yl)-piperidine-4- carboxylic acid 3-chloro-4- methoxy- benzylamide hydrochloride | 1. Method YY1 using the product from preparation P and 3-chloro-4- methoxy- benzylamine as coupling partners 2. Method YY2 3. Method YY3 | ¹H NMR (DMSO- d6): 11.74 (1 H, s), 8.99 (1 H, t), 8.25 (2 H, br s), 8.17 (1 H, s), 7.31 (1 H, d), 7.25-7.15 (2 H, m), 7.09 (1 H, d), 6.66 (1 H, d), 4.53- 4.38 (2 H, m), 4.25 (2 H, d), 3.83 (3 H, s), 3.79-3.64 (2 H, m), 2.32-2.17 (2 H, m), 1.95-1.77 (2 H, m). | M/z: 415 |
| 42 | 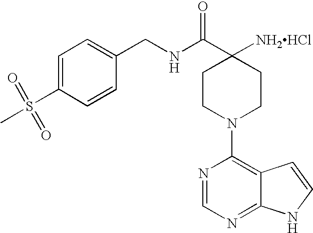 | 4-amino-1-(7H- pyrrolo[2,3- d]pyrimidin-4- yl)-piperidine-4- carboxylic acid 4- methanesulfonyl- benzylamide hydrochloride | 1. Method YY1 using the product from preparation P and 4- methanesulfonyl- benzylamine hydrochloride as coupling partners 2. Method YY2 3. Method YY3 | ¹H NMR (DMSO- d6): 11.75 (1 H, s), 9.20 (1 H, t), 8.48 (2 H, br s), 8.18 (1 H, s), 7.88 (2 H, d), 7.53 (2 H, d), 7.21 (1 H, d), 6.66 (1 H, d), 4.51-4.34 (4 H, m), 3.85-3.70 (2 H, m), 3.19 (3 H, s), 2.37-2.23 (2 H, m), 1.99-1.85 (2 H, m). | M/z: 429 |
| 43 | 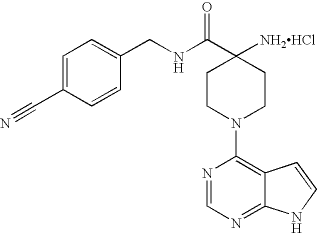 | 4-amino-1-(7H- pyrrolo[2,3- d]pyrimidin-4- yl)-piperidine-4- carboxylic acid 4-cyano- benzylamide hydrochloride | 1. Method YY1 using the product from preparation P and 4- aminomethyl- benzonitrile hydrochloride as coupling partners 2. Method YY2 3. Method YY3 | ¹H NMR (DMSO- d6): 11.75 (1 H, s), 9.19 (1 H, t), 8.61 (2 H, br s), 8.18 (1 H, s), 7.80 (2 H, d), 7.45 (2 H, d), 7.26-7.19 (1 H, m), 6.70-6.63 (1 H, m), 4.50-4.36 (4 H, m), 3.82-3.68 (2 H, m), 2.37-2.23 (2 H, m), 2.00-1.86 (2 H, m). | M/z: 376 |

### Biological Activity

### Example 44

### Measurement of PKA Kinase Inhibitory Activity (IC₅₀)

Compounds of the invention can be tested for PK inhibitory activity using the PKA catalytic domain from Upstate Biotechnology (#14-440) and the 9 residue PKA specific peptide (GRTGRRNSI), also from Upstate Biotechnology (#12-257), as the substrate. A final concentration of 1 nM enzyme is used in a buffer that includes 20 mM MOPS pH 7.2, 40 μM ATP/γ³³P-ATP and 50 mM substrate. Compounds are added in dimethylsulphoxide (DMSO) solution to a final DMSO concentration of 2.5%. The reaction is allowed to proceed for 20 minutes before addition of excess orthophosphoric acid to quench activity. Unincorporated γ³³P-ATP is then separated from phosphorylated proteins on a Millipore MAPH filter plate. The plates are washed, scintillant is added and the plates are then subjected to counting on a Packard Topcount.

The % inhibition of the PKA activity is calculated and plotted in order to determine the concentration of test compound required to inhibit 50% of the PKA activity (IC₅₀).

Following the protocol described above, the IC₅₀ values of the compounds of Examples 3, 4, 6, 7 and 12 have been found to be less than 10 μM.

### Example 45

### Measurement of PKB Kinase Inhibitory Activity (IC₅₀)

The inhibition of protein kinase B (PKB) activity by compounds can be determined essentially as described by Andjelkovic et al. (Mol. Cell. Biol. 19, 5061-5072 (1999)) but using a fusion protein described as PKB-PIF and described in full by Yang et al (Nature Structural Biology 9, 940-944 (2002)). The protein is purified and activated with PDK1 as described by Yang et al. The peptide AKTide-2T (H-A-R-K-R-E-R-T-Y-S-F-G-H-H-A-OH) obtained from Calbiochem (#123900) is used as a substrate. A final concentration of 0.6 nM enzyme is used in a buffer that includes 20 mM MOPS pH 7.2, 30 μM ATP/γ³³P-ATP and 25 μM substrate. Compounds are added in DMSO solution to a final DMSO concentration of 2.5%. The reaction is allowed to proceed for 20 minutes before addition of excess orthophosphoric acid to quench activity. The reaction mixture is transferred to a phosphocellulose filter plate where the peptide binds and the unused ATP is washed away. After washing, scintillant is added and the incorporated activity measured by scintillation counting.

The % inhibition of the PKB activity is calculated and plotted in order to determine the concentration of test compound required to inhibit 50% of the PKB activity (IC₅₀).

Following the protocol described above, the IC₅₀ values of the compounds of Examples 1 to 13 have been found to be less than 20 μM whilst the compounds of Examples 2 to 13 each have IC₅₀ values of less than 1 μM.

### Example 46

### Anti-Proliferative Activity

The anti-proliferative activities of compounds of the invention are determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. All cell lines are obtained from ECACC (European Collection of cell Cultures) or ATCC.

### Pharmaceutical Formulations

### Example 47

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### v) Injectable formulation III

A formulation for i.v. delivery by injection or infusion can be prepared by dissolving the compound of formula (I) (e.g. in a salt form) in water at 20 mg/ml. The vial is then sealed and sterilised by autoclaving.

### vi) Injectable Formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

### (vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### viii) Lyophilised Formulation

Aliquots of formulated compound of formula (I) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-111. (canceled)

112. A compound of the formula (I): 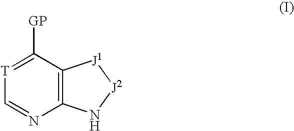
or salts, solvates, tautomers or N-oxides thereof, wherein
(1) GP is a group GP1: 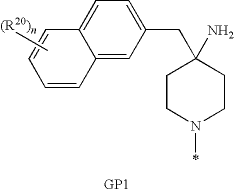
wherein the point of attachment to the bicyclic group is denoted by the asterisk;
R²⁰ is selected from fluorine, chlorine, C₁₋₄ alkoxy, trifluoromethyl, trifluoromethoxy, difluoromethoxy and C₁₋₄ alkyl; n is 0, 1 or 2; T is N; and J¹-J² is CH═CH; or
(2) GP is a group GP2: 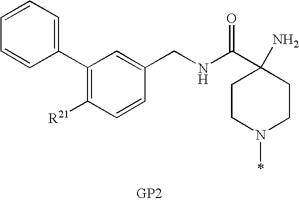
wherein the point of attachment to the bicyclic group is denoted by the asterisk;
R²¹ is selected from fluorine, chlorine, methoxy, trifluoromethyl, trifluoromethoxy, difluoromethoxy and methyl; T is N; and J¹-J² is CH═CH; or
(2A) GP is a group GP2A: 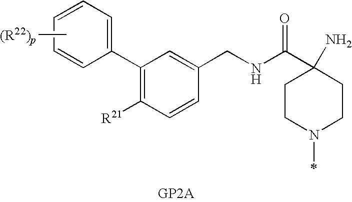
wherein the point of attachment to the bicyclic group is denoted by the asterisk;
R²¹ is selected from fluorine, chlorine, methoxy, trifluoromethyl, trifluoromethoxy, difluoromethoxy and methyl; R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH; or
(3) GP is a group GP3: 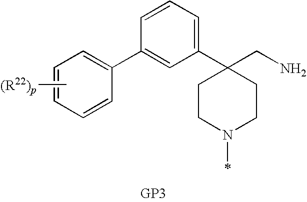
wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH; or
(3A) GP is a group GP3A: 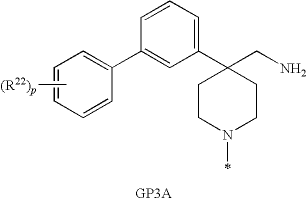
wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH, CH═N or CH₂CO; or
(3B) GP is a group GP3B: 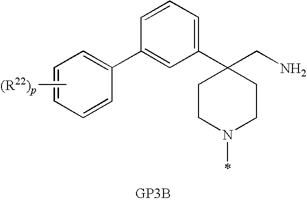
wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH, CH═N or CH₂CO; or
(3C) GP is a group GP3C: 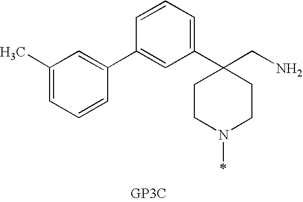
wherein the point of attachment to the bicyclic group is denoted by the asterisk;
T is CH; and J¹-J² is CH₂CO; or
(4) GP is a group GP4: 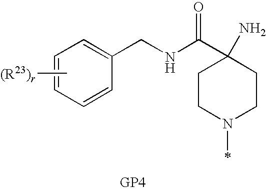
wherein the point of attachment to the bicyclic group is denoted by the asterisk;
R²³ is selected from fluorine; chlorine; C₁₋₄ alkoxy; trifluoromethyl; trifluoromethoxy;
difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); or
(5) GP is a group GP5: 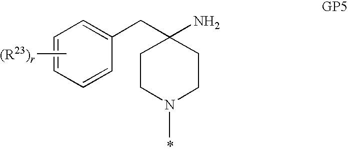
wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); or
(6) GP is a group GP6: 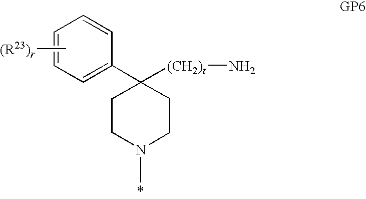
wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2; t is 0 or 1; T is N; and J¹-J² is HN—C(O); provided that when r is 2, no more than 1 substituent R²³ can be an optionally substituted phenyl group; and provided also that when t is 1, r is 1 and R²³ is other than a 4-chloro substituent; or
(7) GP is a group GP7: 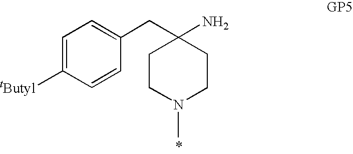
wherein the point of attachment to the bicyclic group is denoted by the asterisk;
T is N; and J¹-J² is CH═N; or
(8) GP is a group GP8: 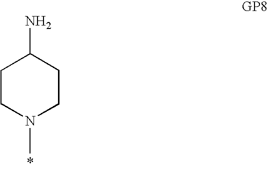
wherein the point of attachment to the bicyclic group is denoted by the asterisk;
T is CH; and J¹-J² is CH₂—C(O); or
(9) GP is a group GP9: 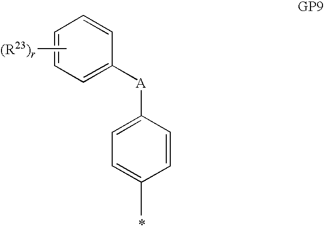
wherein the point of attachment to the bicyclic group is denoted by the asterisk;
R²³ is selected from fluorine; chlorine; C₁₋₄ alkoxy; trifluoromethyl; trifluoromethoxy;
difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); and A is selected from:
(i) 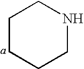
wherein the letter “a” denotes the point of attachment to the neighbouring benzene rings;
(ii) CH—CH₂—NHCH₃;
(iii) CH—CH₂—CH₂—NH₂; and
(iv) C(OH)—CH₂—CH₂—NH₂.
(10) GP is a group GP10: 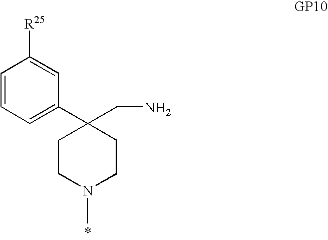
wherein the point of attachment to the bicyclic group is denoted by the asterisk;
R²⁵ is selected from hydrogen, fluorine; chlorine; C₁₋₄ alkoxy; trifluoromethyl; trifluoromethoxy; difluoromethoxy; and C₁₋₄ alkyl; T is CH or N; and J¹-J² is CH₂CO or CH═N; or
(11)GP is a group GP11: 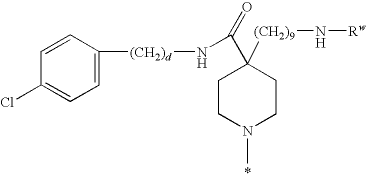
wherein (a) g is 0; d is 1; R^{w} is hydrogen or methyl; T is N; J¹-J² is N═CH, CH₂CO or CH═N; or (b) g is 1; d is 0 or 1; R^{w} is hydrogen; T is N; and J¹-J² is CH═CH; or
(12) GP is a group GP12: 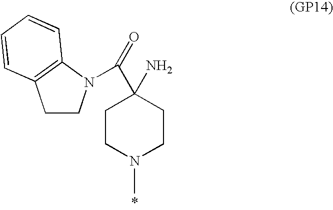
wherein T is N and J¹-J² is CH═CH; or
(13) GP is a group GP13: 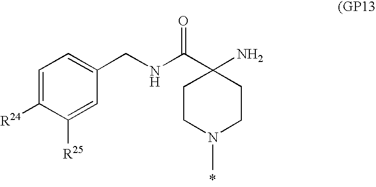
wherein T is N and J¹-J² is CH═CH; and (a) R²⁴ is methoxy and R²⁵ is hydrogen or chlorine; or (b) R²⁴ is methanesulphonyl or cyano and R²⁵ is hydrogen or
(14) GP is a group GP14: 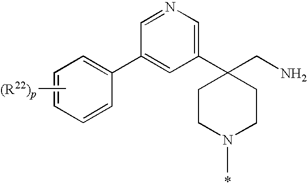
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH.

113. A compound according to claim 112 having the formula (IA): 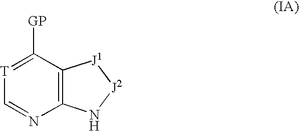
or salts, solvates, tautomers or N-oxides thereof, wherein
(iv) GP is a group GP1: 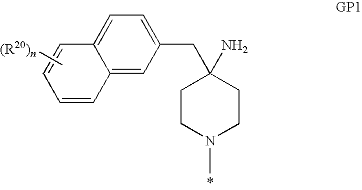
wherein the point of attachment to the bicyclic group is denoted by the asterisk;
R²⁰ is selected from fluorine, chlorine, C₁₋₄ alkoxy, trifluoromethyl, trifluoromethoxy, difluoromethoxy and C₁₋₄ alkyl; n is 0, 1 or 2; T is N; and J¹-J² is CH═CH; or
(2) GP is a group GP2: 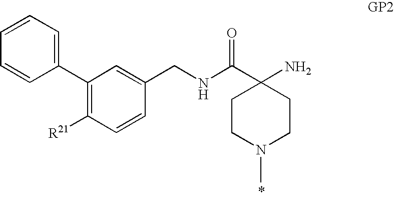
wherein the point of attachment to the bicyclic group is denoted by the asterisk;
R²¹ is selected from fluorine, chlorine, methoxy, trifluoromethyl, trifluoromethoxy, difluoromethoxy and methyl; T is N; and J¹-J² is CH═CH; or
(3) GP is a group GP3: 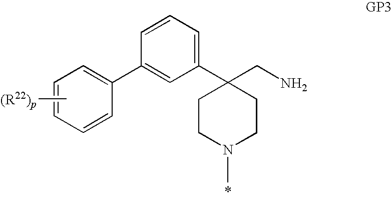
wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH; or
(4) GP is a group GP4: 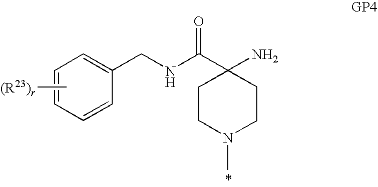
wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); or
(5) GP is a group GP5: 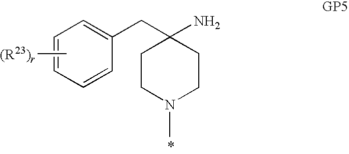
wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O); or
(6) GP is a group GP6: 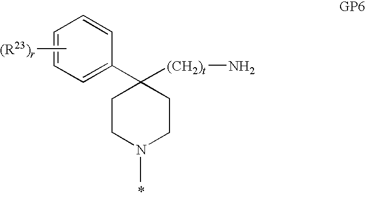
wherein the point of attachment to the bicyclic group is denoted by the asterisk;
R²³ is selected from fluorine; chlorine; C₁₋₄ alkoxy; trifluoromethyl; trifluoromethoxy, difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2; t is 0 or 1; T is N; and J¹-J² is HN—C(O); provided that when r is 2, no more than 1 substituent R²³ can be an optionally substituted phenyl group; and provided also that when t is 1, r is 1 and R²³ is other than a 4-chloro substituent; or
(7) GP is a group GP7: 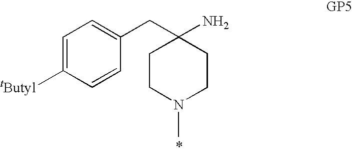
wherein the point of attachment to the bicyclic group is denoted by the asterisk;
T is N; and J¹-J² is CH═N; or
(8) GP is a group GP8: 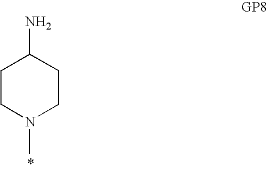
wherein the point of attachment to the bicyclic group is denoted by the asterisk;
T is CH; and J¹-J² is CH₂—C(O); or
(9) GP is a group GP9: 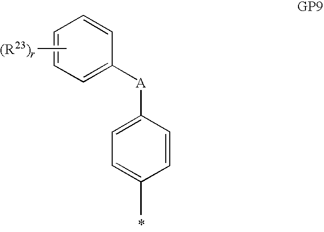
wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; t is 0 or 1; T is N; and J¹-J² is HN—C(O); and A is selected from:
(i) 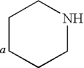
wherein the letter “a” denotes the point of attachment to the neighbouring benzene rings;
(ii) CH—CH₂—NHCH₃;
(iii) CH—CH₂—CH₂—NH₂; and
(iv) C(OH)—CH₂—CH₂—NH₂.

114. A compound according to claim 112 wherein the moiety GP is a group GP1: 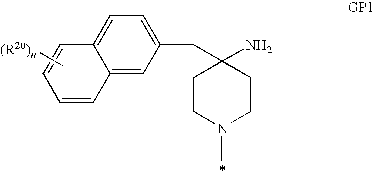
or salts, solvates, tautomers or N-oxides thereof,
wherein the point of attachment to the bicyclic group is denoted by the asterisk;
R²⁰ is selected from fluorine, chlorine, C₁₋₄ alkoxy, trifluoromethyl, trifluoromethoxy, difluoromethoxy and C₁₋₄ alkyl; n is 0, 1 or 2; T is N; and J¹-J² is CH═CH.

115. A compound according to claim 112 wherein the moiety GP is a group GP2: 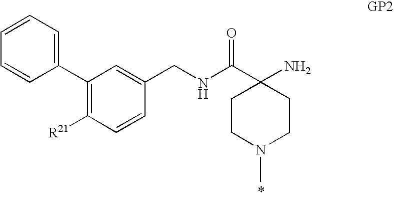
or salts, solvates, tautomers or N-oxides thereof, wherein
the point of attachment to the bicyclic group is denoted by the asterisk;
R²¹ is selected from fluorine, chlorine, methoxy, trifluoromethyl, trifluoromethoxy, difluoromethoxy and methyl; T is N; and J¹-J² is CH═CH.

116. A compound according to claim 112 wherein GP is a group GP2A: 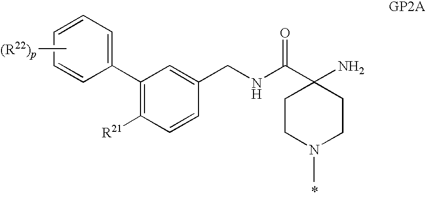
or salts, solvates, tautomers or N-oxides thereof,
wherein the point of attachment to the bicyclic group is denoted by the asterisk;
R²¹ is selected from fluorine, chlorine, methoxy, trifluoromethyl, trifluoromethoxy, difluoromethoxy and methyl; R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH.

117. A compound according to claim 112 wherein the moiety GP is a group GP3: 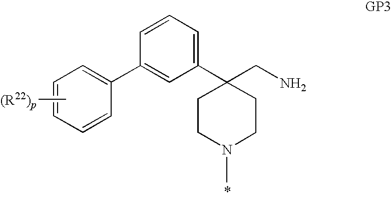
or salts, solvates, tautomers or N-oxides thereof,
wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is CH═CH.

118. A compound according to claim 112 wherein GP is a group GP3A: 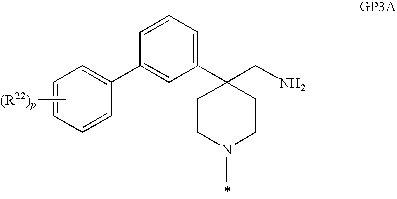
or salts, solvates, tautomers or N-oxides thereof,
wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH, CH═N or CH₂CO.

119. A compound according to claim 112 wherein the moiety GP is a group GP4: 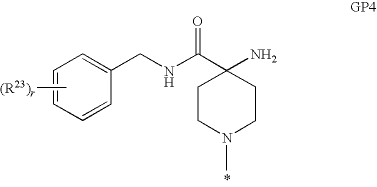
wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O).

120. A compound according to claim 112 wherein the moiety GP is a group GP5: 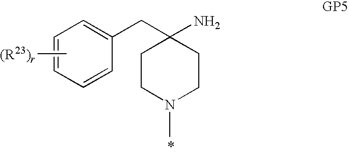
wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2, provided that when r is 2, no more than 1 substituent R²³ can be an optionally substituted phenyl group; T is N; and J¹-J² is HN—C(O).

121. A compound according to claim 112 wherein the moiety GP is a group GP6: 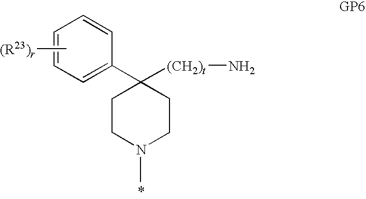
wherein the point of attachment to the bicyclic group is denoted by the asterisk;
R²³ is selected from fluorine; chlorine; C₁₋₄ alkoxy; trifluoromethyl; trifluoromethoxy; difluoromethoxy; C₁₋₄ alkyl; and phenyl optionally substituted by fluorine, chlorine, methoxy, trifluoromethyl, trifluoromethoxy, difluoromethoxy or methyl; r is 0, 1 or 2; t is 0 or 1; T is N; and J¹-J² is HN—C(O); provided that when r is 2, no more than 1 substituent R²³ can be an optionally substituted phenyl group; and provided also that when t is 1, r is 1 and R²³ is other than a 4-chloro substituent.

122. A compound according to claim 112 wherein the moiety GP is a group GP7: 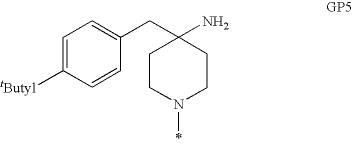
wherein the point of attachment to the bicyclic group is denoted by the asterisk;
T is N; and J¹-J² is CH═N.

123. A compound according to claim 112 wherein GP is a group GP10: 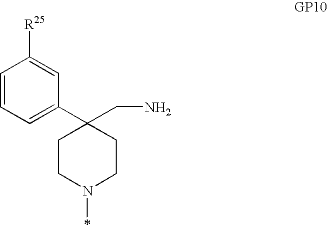
wherein the point of attachment to the bicyclic group is denoted by the asterisk;
R²⁵ is selected from hydrogen, fluorine; chlorine; C₁₋₄ alkoxy; trifluoromethyl; trifluoromethoxy; difluoromethoxy; and C₁₋₄ alkyl; T is CH or N; and J¹-J² is CH₂CO or CH═N.

124. A compound according to claim 112 wherein GP is a group GP11: 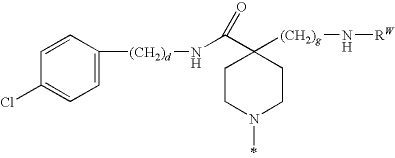
wherein (a) g is 0; d is 1; R^{w} is hydrogen or methyl; T is N; J¹-J² is N═CH, CH₂CO or CH═N; or (b) g is 1; d is 0 or 1; R^{w} is hydrogen; T is N; and J¹-J² is CH═CH.

125. A compound according to claim 112 wherein GP is a group GP13: 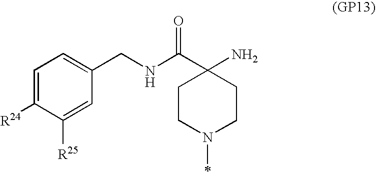
wherein T is N and J¹-J² is CH═CH; and (a) R²⁴ is methoxy and R²⁵ is hydrogen or chlorine; or (b) R²⁴ is methanesulphonyl or cyano and R²⁵ is hydrogen.

126. A compound according to claim 112 wherein GP is a group GP14: 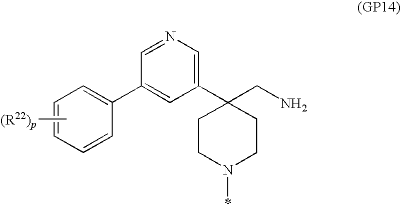
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH.

127. A compound according to claim 112 wherein GP is a group GP3B: 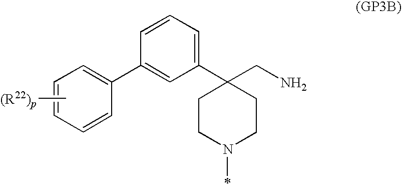
wherein the point of attachment to the bicyclic group is denoted by the asterisk;
R²² is selected from fluorine, chlorine, C₁₋₄ alkoxy, trifluoromethyl, trifluoromethoxy, difluoromethoxy and C₁₋₄ alkyl; p is 0, 1 or 2; T is N; and J¹-J² is N═CH, CH═N or CH₂CO.

128. A compound as defined in claim 112 in the form of a salt, solvate or N-oxide.

129. A method for the prophylaxis or treatment of a disease state or condition mediated by protein kinase A or protein kinase B, which method comprises administering to a subject in need thereof a compound as defined in claim 112.

130. A method for treating a disease or condition comprising or arising from abnormal cell growth or abnormally arrested cell death in a mammal, which method comprises administering to the mammal a compound as defined in claim 112 in an amount effective in inhibiting abnormal cell growth.

131. A pharmaceutical composition comprising a compound as defined in claim 112 and a pharmaceutically acceptable carrier.
